(19) United States
(12) Patent Application Publication
(10) Pub. No.: US 2016/0338976 A1
(43) Pub. Date: Nov. 24, 2016

(54) TAMPER RESISTANT DOSAGE FORM WITH BIMODAL RELEASE PROFILE MANUFACTURED BY CO-EXTRUSION

(71) Applicant: GRÜNENTHAL GMBH, AACHEN (DE)

(72) Inventors: ANJA GEIßLER, KARLSRUHE (DE); KLAUS WENING, KOLN (DE); LUTZ BARNSCHEID, MONCHENGLADBACH (DE); JANA PÄTZ, BORNHEIM (DE)

(73) Assignee: GRÜNENTHAL GMBH, AACHEN (DE)

(21) Appl. No.: 15/229,531

(22) Filed: Aug. 5, 2016

Related U.S. Application Data
(63) Continuation of application No. 14/569,817, filed on Dec. 15, 2014.

(30) Foreign Application Priority Data
Dec. 16, 2013 (EP) 13197503.9

(51) Int. Cl. A61K 31/135; A61K 9/28; A61K 31/167; A61K 9/24; A61K 9/20

(52) U.S. Cl. CPC A61K 31/135; A61K 9/209; A61K 9/2095; A61K 31/167; A61K 9/2031; A61K 9/2054; A61K 9/28

(57) ABSTRACT

Summary:

The invention relates to a monolithic pharmaceutical dosage form comprising a hot melt-extruded first segment (S₁) and a second segment (S₂); wherein the first segment (S₁) contains at least a first pharmacologically active ingredient (A₁) and/or the second segment (S₂) contains at least a second pharmacologically active ingredient (A₂); and the segment (S₁) and/or the segment (S₂) is tamper-resistant and/or exhibits a breaking strength of at least 300 N.

This application is a division of U.S. patent application Ser. No. 14/569,817, filed Dec. 15, 2014, which claims priority of European Patent Application No. 13197503.9, filed on Dec. 16, 2013, the entire contents of which are incorporated herein by reference.

The invention relates to a monolithic pharmaceutical dosage form comprising a hot melt-extruded first segment (S₁) and a second segment (S₂); wherein the first segment (S₁) contains at least a first pharmacologically active ingredient (A₁) and/or the second segment (S₂) contains at least a second pharmacologically active ingredient (A₂); and the segment (S₁) and/or the segment (S₂) is tamper-resistant and/or exhibits a breaking strength of at least 300 N.

### BACKGROUND OF THE INVENTION

A large number of pharmacologically active substances have a potential for being abused or misused, i.e. they can be used to produce effects which are not consistent with their intended use. Thus, e.g. opioids which exhibit an excellent efficacy in controlling severe to extremely severe pain are frequently abused to induce euphoric states similar to being intoxicated. In particular, active substances which have a psychotropic effect are abused accordingly.

To enable abuse, the corresponding pharmaceutical dosage forms, such as pharmaceutical dosage forms or capsules are crushed, for example ground by the abuser, the active substance is extracted from the thus obtained powder using a preferably aqueous liquid and after being optionally filtered through cotton wool or cellulose wadding, the resultant solution is administered parenterally, in particular intravenously. This type of dosage results in an even faster diffusion of the active substance compared to the oral abuse, with the result desired by the abuser, namely the kick. This kick or these intoxication-like, euphoric states are also reached if the powdered pharmaceutical dosage form is administered nasally, i.e. is sniffed.

Various concepts for the avoidance of drug abuse have been developed.

It has been proposed to incorporate in pharmaceutical dosage forms aversive agents and/or antagonists in a manner so that they only produce their aversive and/or antagonizing effects when the pharmaceutical dosage forms are tampered with. However, the presence of such aversive agents is principally not desirable and there is a need to provide sufficient tamper resistance without relying on aversive agents and/or antagonists.

Another concept to prevent abuse relies on the mechanical properties of the pharmaceutical dosage forms, particularly an increased breaking strength (resistance to crushing). The major advantage of such pharmaceutical dosage forms is that comminuting, particularly pulverization, by conventional means, such as grinding in a mortar or fracturing by means of a hammer, is impossible or at least substantially impeded. Thus, the pulverization, necessary for abuse, of the pharmaceutical dosage forms by the means usually available to a potential abuser is prevented or at least complicated. Such pharmaceutical dosage forms are useful for avoiding drug abuse of the pharmacologically active ingredient contained therein, as they may not be powdered by conventional means and thus, cannot be administered in powdered form, e.g. nasally. The mechanical properties, particularly the high breaking strength of these pharmaceutical dosage forms renders them tamper resistant. In the context of such tamper-resistant pharmaceutical dosage forms it can be referred to, e.g., WO 2005/016313, WO 2005/016314, WO 2005/063214, WO 2005/102286, WO 2006/002883, WO 2006/002884, WO 2006/002886, WO 2006/082097, WO 2006/082099 and WO2009/092601.

Besides tampering of pharmaceutical dosage forms in order to abuse the drugs contained therein, the potential impact of concomitant intake of ethanol on the in vivo release of drugs from modified release oral formulations (dose dumping) has recently become an increasing concern. Controlled or modified release formulations typically contain a higher amount of the pharmacologically active ingredient relative to its immediate release counterpart. If the controlled release portion of the formulation is easily defeated, the end result is a potential increase in exposure to the active drug and possible safety concerns. In order to improve safety and circumvent intentional tampering (e.g. dissolving a controlled release pharmaceutical dosage form in ethanol to extract the drug), a reduction in the dissolution of the modified release fractions of such formulations, in ethanol, may be of benefit. Accordingly, the need exists to develop new formulations having reduced potential for dose dumping in alcohol.

Furthermore, the release kinetics of the pharmacologically active ingredients is an important factor. It is well known that depending on how a pharmaceutically pharmacologically active ingredient is formulated into a tablet its release pattern can be modified.

On the one hand, formulations providing immediate release upon oral administration have the advantage that they lead to a fast release of the pharmacologically active ingredient in the gastrointestinal tract. As a result, a comparatively high dose of the pharmacologically active ingredient is quickly absorbed leading to high plasma levels within a short period of time and resulting in a rapid onset of medicinal action, i.e. medicinal action begins shortly after administration. At the same time, however, a rapid reduction in the medicinal action is observed, because metabolization and/or excretion of the pharmacologically active ingredient cause a decrease of plasma levels. For that reason, formulations providing immediate release of pharmacologically active ingredients typically need to be administered frequently, e.g. six times per day. This may cause comparatively high peak plasma pharmacologically active ingredient concentrations and high fluctuations between peak and trough plasma pharmacologically active ingredient concentrations which in turn may deteriorate tolerability.

Controlled release (e.g. delayed release, prolonged release, sustained release, and the like) may be based upon various concepts such as coating the pharmaceutical dosage form with a controlled release membrane, embedding the pharmacologically active ingredient in a matrix, binding the pharmacologically active ingredient to an ion-exchange resin, forming a complex of the pharmacologically active ingredient, and the like. In this context it can be referred to, e.g., W. A. Ritschel, Die Tablette, 2. Auflage, Editio Cantor Verlag Aulendorf, 2002.

In comparison to formulations providing immediate release, formulations providing prolonged release upon oral administration have the advantage that they need to be administered less frequently, typically once daily or twice daily. This can reduce peak plasma pharmacologically active ingredient concentrations and fluctuations between peak and trough plasma pharmacologically active ingredient concentrations which in turn may improve tolerability.

However, especially patients starting their treatment with controlled release formulations often desire a rapid onset of medicinal action. Therefore, a need exists to develop tamper resistant formulations which provide a quick medicinal action while at the same time having the benefits of controlled or modified release formulations.

WO 03/024430 relates to a pharmaceutical composition for controlled release of an active substance, wherein the active substance is released into an aqueous medium by erosion of at least one surface of the composition. The composition comprises i) a matrix comprising a) polymer or a mixture of polymers, b) an active substance and, optionally, c) one or more pharmaceutically acceptable excipients, and ii) a coating. Zero order release is desirable. The matrix typically comprises PEO and the active substance is typically an opioid such as morphine or a glucuronide thereof. The coating comprises a first cellulose derivative which is substantially insoluble in the aqueous medium and at least one of a) a second cellulose derivative which is soluble or dispersible in water, b) a plasticizer, and, d) a filler.

Pharmaceutical dosage forms providing controlled release of an active ingredient are also known from WO 2010/149169, WO 2004/084869, US 2005/089569, WO 2008/086804, WO 2010/088911, WO 2010/083843, WO 2008/148798 and WO 2006/128471.

L. Dierickxs et al. disclose the manufacture of a core/coat dosage form by co-extrusion, wherein the core provides sustained drug release of metoprolol tartrate and the coat immediate drug release of hydrochlorothiazide (L. Dierickxs et al., Eur. J. Pharm. Biopharm. 2012, 81, 683-689; L. Dierickxs et al., Co-extrusion as manufacturing technique for fixed-dose combination mini-tablets, poster displayed at AAPS annual meeting 2011).

U. Quintavalle et al. disclose the preparation of sustained release co-extrudates by hot-melt extrusion, wherein the inner extruded matrix has a hydrophilic character and the outer extruded matrix has a lipophilic character and wherein both matrices contained theophylline (U. Quintavalle et al., Eur. J. Pharm. Sci. 2008, 33, 282-293).

G. C. Oliveira et al. disclose laminar coextrudates manufactured at room temperature which are composed of three layers, wherein the model drug coumarin is only included in the inner layer (G. C. Oliveira et al., Production and characterization of laminar coextrudates at room temperature in the absence of solvents, poster displayed at AAPS annual meeting 2012).

US 2009/0022798 discloses formulations and methods for the delivery of drugs, particularly drugs of abuse, having an abuse-relevant drug substantially confined in the core and a non-abuse relevant drug in a non-core region. These formulations have reduced potential for abuse. In the formulation, preferably the abuse relevant drug is an opioid and the non-abuse relevant drug is acetaminophen or ibuprofen. More preferably, the opioid is hydrocodone, and the non-abuse relevant analgesic is acetaminophen. In certain preferred embodiments, the dosage forms are characterized by resistance to solvent extraction; tampering, crushing or grinding. Certain embodiments relate to dosage forms providing an initial burst of release of drug followed by a prolonged period of controllable drug release. When providing these dosage forms with tamper resistant properties, however, the initial burst of release of drug is difficult to achieve, as tamper-resistance typically relies on the presence of polymers that act as release matrix material slowing down the release of the drug from the dosage form. The non-core layer of said drug product is explicitly applied using a film-coating process. A film-coating process is disadvantageous due to the high cost it produces during manufacturing. The film-forming layer material is first dissolved, then sprayed on the core and finally the solvent is removed, all leading to long process times with high energy consumption. Due to the high amount of active that needs to be present in the film-layer, this is a significant disadvantage for a cost-competitive manufacturing of the drug product.

US 2010/172989 relates to at least one abuse-resistant drug delivery composition for delivering a drug having potential for dose dumping in alcohol, related methods of preparing these dosage forms, and methods of treating a patient in need thereof comprising administering the compositions to the patient.

US 2013/303623 discloses a thermoformed, tamper-resistant pharmaceutical dosage form comprising: a) a pharmacologically active ingredient; b) a polyalkylene oxide having a weight average molecular weight of more than 200,000 g/mol; and c) a zinc component, wherein the content of said zinc component is at least 1 ppm, relative to the total weight of the pharmaceutical dosage form.

WO 2008/132707 relates to an extrusion process comprising extruding a material that is flowable when heated and passing the extrudate thus formed through a nozzle 10 to shape the extrudate into a plurality of substantially uniformly shaped elements such as minispheres or minicapsules.

US 2010/104638 discloses an extended release oral administered dosage form of acetaminophen and tramadol. The dosage form includes a composition of acetaminophen together with a tramadol complex formed with an anionic polymer. The tramadol complex provides sustained release of tramadol for a synchronized (coordinated) release profile of acetaminophen and tramadol.

The properties of the pharmaceutical dosage forms of the prior art are not satisfactory in every respect.

It is an object of the invention to provide pharmaceutical dosage forms which have advantages over the pharmaceutical dosage forms of the prior art. The pharmaceutical dosage forms should provide prolonged or immediate release of a first pharmacologically active ingredient and prolonged or immediate release of a second pharmacologically active ingredient, wherein the first pharmacologically active ingredient and/or the second pharmacologically active ingredient is safeguarded from abuse.

This object has been achieved by the subject-matter described hereinbelow.

A first aspect of the invention relates to a monolithic pharmaceutical dosage form comprising
a hot melt-extruded first segment (S₁); and
a second segment (S₂);
wherein
the first segment (S₁) contains at least a first pharmacologically active ingredient (A₁) and/or the second segment (S₂) contains at least a second pharmacologically active ingredient (A₂); and
the segment (S₁) and/or the segment (S₂) is tamper-resistant and/or exhibits a breaking strength of at least 300 N.

In a particularly preferred embodiment, the monolithic pharmaceutical dosage form according to the invention comprises
a hot melt-extruded first segment (S₁) containing a first pharmacologically active ingredient (A₁); and
a hot melt-extruded second segment (S₂) containing a second pharmacologically active ingredient (A₂);
wherein
the segment (S₁) and/or the segment (S₂) is tamper-resistant and/or exhibits a breaking strength of at least 300 N; and
the segment (S₁) and/or the segment (S₂) provides prolonged release of the pharmacologically active ingredient (A₁) or (A₂) contained therein.

Another aspect of the invention relates to a process for the production of said monolithic pharmaceutical dosage form comprising the steps of
(i) hot melt-extruding a first segment (S₁) preferably containing a first pharmacologically active ingredient (A₁); and
(ii) preferably hot melt-extruding a second segment (S₂) preferably containing a second pharmacologically active ingredient (A₂);
   wherein step (i) is performed before, after and/or simultaneously with step (ii).

### BRIEF DESCRIPTION OF THE DRAWINGS

The invention will now be described in greater detail with reference to the drawings, wherein:

FIG. 1A schematically illustrates a two-layer tablet comprising a first segment (S₁) as first layer (1) and a second segment (S₂) as second layer (2).

FIG. 1B schematically illustrates a mantle tablet comprising a first segment (S₁) as a core (3) and a second segment (S₂) (4) surrounding said core (3).

FIG. 1C schematically illustrates a three-layer tablet comprising a first segment (S₁) as first layer (5) and two second segments (S₂) as layer (6) and layer (7).

FIG. 1D schematically illustrates a mantled three-layer tablet comprising a first segment (S₁) as first layer (5) and mantle (8) and two second segments (S₂) as layer (6) and layer (7).

FIG. 2 shows the release profile of the embodiment of Example 1 hereinbelow.

FIG. 3 shows the release profile of the embodiment of Example 2 hereinbelow.

FIG. 4 shows the release profile of the embodiment of Example 3 hereinbelow.

FIG. 5 shows the release profile of the embodiment of Example 4 hereinbelow.

FIG. 6 shows the release profile of the embodiment of Example 5 hereinbelow.

FIG. 7 shows the release profile of the embodiment of Example 6 hereinbelow.

FIG. 8 shows the release profile of the embodiment of Example 7 hereinbelow.

FIG. 9 shows the release profile of the embodiment of Example 8 hereinbelow.

FIG. 10 shows the release profile of the embodiment of Example 9 hereinbelow.

FIG. 11 shows the release profile of the embodiment of Example 10 hereinbelow.

FIG. 12 shows the release profile of the embodiment of Example 11 hereinbelow.

FIG. 13 shows the release profile of the embodiment of Example 12 hereinbelow.

It has been surprisingly found that tamper-resistant monolithic pharmaceutical dosage forms preferably having a bimodal release profile which preferably contain a first pharmacologically active ingredient and a second pharmacologically active ingredient can be prepared by hot melt extrusion. Further, the manufacture of the monolithic pharmaceutical dosage forms is cost-effective and ensures a consistently high quality. Still further, patient compliance can be improved by providing a rapid but also prolonged medicinal effect.

Unless expressly stated otherwise, all percentages are by weight (wt.-%).

For the purpose of specification, the term “pharmaceutical dosage form” refers to a pharmaceutical entity which contains the first pharmacologically active ingredient (A₁) and/or the second pharmacologically active ingredient (A₂) and which is to be administered to a patient (dose unit). It may be molded during manufacture, and it may be of almost any size, shape, weight, and color. Preferably, the monolithic pharmaceutical dosage form is solid or semi-solid.

For the purpose of specification, the term “monolithic” means non-multiparticulate. Thus, the monolithic pharmaceutical dosage form according to the present invention is single body or single entity which does not comprise any plurality of particles. In this regard, the monolithic pharmaceutical dosage form is neither a filled capsule nor a compressed tablet which comprises one or more matrix-embedded particles. Nonetheless, the monolithic pharmaceutical dosage form according to the present invention can comprise different elements such as layers, sections or a film coating.

The monolithic pharmaceutical dosage form is preferably intended for oral administration. It is preferably provided in form of a single body that can be easily swallowed by a patient. Typical examples of pharmaceutical dosage forms according to the invention include, but are not limited to tablets (e.g. mantle tablets, layered tablets and film-coated tablets).

For the purpose of specification, the term “segment” as used herein refers to any preferably hot melt-extruded physically distinct entity of the monolithic pharmaceutical dosage form that preferably contains the first pharmacologically active ingredient (A₁) or the second pharmacologically active ingredient (A₂) and that can be distinguished from another physically distinct entity of the pharmaceutical dosage form. Preferably, every segment is solid or semi-solid.

The first segment (S₁) is hot melt extruded. The second segment (S₂) is preferably hot melt extruded but can also be manufactured by other means than hot melt extrusion. A person skilled in the art knows manufacturing methods besides hot melt extrusion, such as e.g. granulation or direct compression. When the second segment (S₂) is not hot melt extruded, it preferably has a thickness of at least 200 μm, more preferably at least 300 μm, still more preferably at least 400 μm, yet more preferably at least 500 μm, even more preferably at least 600 μm, most preferably at least 700 μm or at least 800 μm and in particular at least 900 μm, at least 1,000 μm or at least 1,500 μm. In another preferred embodiment, when the second segment (S₂) is not hot melt extruded, the second segment (S₂) is not a film coating.

For the purpose of specification, a film coating preferably does not contain any pharmacologically active ingredient and preferably has a thickness of at most 150 μm, more preferably at most 120 μm, still more preferably at most 100 μm, even more preferably at most 80 μm, yet more preferably at most 60 μm, most preferably at most 40 μm and in particular at most 20 μm and does not constitute any segment of the monolithic pharmaceutical dosage form.

In a particularly preferred embodiment, both, the segment (S₁) as well as the segment (S₂), are hot melt extruded.

A skilled person knows how to distinguish a segment and a pharmaceutical dosage form, respectively, which was manufactured by hot melt-extrusion from a segment and a pharmaceutical dosage form, respectively, which was manufactured by direct compression or granulation. Preferred analytical methods which are suitable to distinguish hot melt-extruded segments and hot melt-extruded pharmaceutical dosage forms, respectively, from segments and pharmaceutical dosage forms, respectively, manufactured by direct compression or granulation include X-ray diffraction, scanning electron microscopy, transmission electron microscopy, porosity measurements, near-infrared spectroscopy (NIR), Raman spectroscopy and tetrahertz spectroscopy.

In a preferred embodiment, the first segment (S₁) contains at least a first pharmacologically active ingredient (A₁) and the second segment (S₂) preferably does not contain any pharmacologically active ingredient. In another preferred embodiment, the second segment (S₂) contains at least a second pharmacologically active ingredient (A₂) and the first segment (S₁) preferably does not contain any pharmacologically active ingredient. In still another preferred embodiment, the first segment (S₁) contains at least a first pharmacologically active ingredient (A₁) and a further pharmacologically active ingredient (A_{f}). According to this embodiment, the second segment (S₂) preferably does not contain any pharmacologically active ingredient. In yet another preferred embodiment, the second segment (S₂) contains at least a second pharmacologically active ingredient (A₂) and a further pharmacologically active ingredient (A_{f}). According to this embodiment, the first segment (S₁) preferably does not contain any pharmacologically active ingredient.

In a particularly preferred embodiment, the first segment (S₁) contains at least a first pharmacologically active ingredient (A₁) and the second segment (S₂) contains at least a second pharmacologically active ingredient (A₂). In another particularly preferred embodiment, the first segment (S₁) contains a first pharmacologically active ingredient (A₁) as the only pharmacologically active ingredient and the second segment (S₂) contains a second pharmacologically active ingredient (A₂) as the only pharmacologically active ingredient.

The first segment (S₁) and the second segment (S₂) of the monolithic pharmaceutical dosage form preferably contain the first pharmacologically active ingredient (A₁) and the second pharmacologically active ingredient (A₂), respectively. However, the first segment (S₁) and the second segment (S₂) preferably do not consist of the first pharmacologically active ingredient (A₁) and the second pharmacologically active ingredient (A₂), respectively, but contain further ingredients such as pharmaceutical excipients. Thus, the first segment (S₁) and the second segment (S₂) can be regarded as greater units of preferably hot melt-extruded material, comprising inter alia but not consisting of the first pharmacologically active ingredient (A₁) and the second pharmacologically active ingredient (A₂), respectively. While one segment may partially or completely surround the other segment, it is nevertheless not possible that a given location of the monolithic pharmaceutical dosage form contains both, matter of the first segment (S₁) and simultaneously matter of the second segment (S₂).

Preferably, besides the content of the first pharmacologically active ingredient (A₁) and the second pharmacologically active ingredient (A₂), the first segment (S₁) and the second segment (S₂) of the monolithic pharmaceutical dosage form preferably differ in at least one of the following properties and can be distinguished by said property: composition of ingredients (e.g. nature and/or amount), total weight, density, hardness, breaking strength, size, shape, color, morphology, position within the monolithic pharmaceutical dosage form (e.g. core, mantle, layer) and/or porosity. According to the present invention, preferably neither the first segment (S₁) nor the second segment (S₂) forms a coating of the pharmaceutical dosage form, particularly no spray coating.

In a particularly preferred embodiment, the first segment (S₁) is hot melt-extruded and contains a first pharmacologically active ingredient (A₁) and the second segment (S₂) is hot melt-extruded and contains a second pharmacologically active ingredient (A₂).

Typically, any segment of the monolithic pharmaceutical dosage form covers at least 1 vol.-%, or at least 2 vol.-%, or at least 5 vol.-%, more preferably at least 10 vol.-%, still more preferably at least 15 vol.-%, yet more preferably at least 20 vol.-%, even more preferably at least 25 vol.-%, most preferably at least 30 vol.-%, and in particular at least 35 vol.-%, of the total volume of the pharmaceutical dosage form. Thus, physically distinct entities that are so small that they do not cover such portion of the total volume of the monolithic pharmaceutical dosage form are typically not to be regarded as “segment” in the meaning of the invention.

Preferably, a segment is a spatially confined area within the monolithic pharmaceutical dosage form such as a layer, core or mantle (i.e. shell) of the monolithic pharmaceutical dosage form.

The first segment (S₁) and the second segment (S₂) of the monolithic pharmaceutical dosage form are separate of one another, i.e. they are at different locations of the pharmaceutical dosage form. However, preferably, the first segment (S₁) and the second segment (S₂) are directly adjacent to each other, i.e. they preferably share at least one common boundary.

In a preferred embodiment, the second segment (S₂) covers at least a part of the surface of the first segment (S₁).

Preferably, the second segment (S₂) covers at least 5% or 25% or 45%, more preferably at least 10% or 30% or 50%, still more preferably at least 20% or 40% or 60%, yet more preferably at least 30% or 50% or 70%, even more preferably at least 40% or 60% or 80%, most preferably at least 50% or 70% or 90% and in particular at least 60% or 80% or 99% of the surface of the first segment (S₁).

In another preferred embodiment, the second segment (S₂) covers the entire surface of the first segment (S₁). According to this embodiment, the second segment (S₂) preferably forms a mantle or shell around the first segment (S₁).

The first segment (S₁) and the second segment (S₂) of the monolithic pharmaceutical dosage form can be distinguished from one another.

The monolithic pharmaceutical dosage form according to the invention comprises at least one hot melt-extruded first segment (S₁) (e.g. a layer, core or mantle) but may also contain a plurality of first segments (S₁) (e.g. layers in a layered tablet or the mantle and one or more layers in a mantled layered tablet). When the monolithic pharmaceutical dosage form according to the invention comprises a plurality of first segments (S₁), the individual first segments (S₁) are preferably of essentially the same type and nature, e.g. composition, total weight, density, hardness, breaking strength, size, shape, color, morphology, coherence and/or porosity. Preferably, the monolithic pharmaceutical dosage form contains not more than 10 first segments (S₁), more preferably not more than 9, still more preferably not more than 8, yet more preferably not more than 7, even more preferably not more than 6, most preferably not more than 5, and in particular not more than 4 first segments (S₁). Preferably, the monolithic pharmaceutical dosage form contains 1, 2 or 3, most preferably 1 first segment (S₁).

The monolithic pharmaceutical dosage form according to the invention comprises at least one preferably hot melt-extruded second segment (S₂) (e.g. layer, core or mantle) but may also contain a plurality of second segments (S₂) (e.g. layers in a layered tablet or the mantle and one or more layers in a mantled layered tablet). When the monolithic pharmaceutical dosage form according to the invention comprises a plurality of second segments (S₂), the individual second segments (S₂) are preferably of essentially the same type and nature, e.g. composition, total weight, density, hardness, breaking strength, size, shape, color, morphology, coherence and/or porosity. Preferably, the monolithic pharmaceutical dosage form contains not more than 10 second segments (S₂), more preferably not more than 9, still more preferably not more than 8, yet more preferably not more than 7, even more preferably not more than 6, most preferably not more than 5, and in particular not more than 4 second segments (S₂). Preferably, the monolithic pharmaceutical dosage form contains 1, 2 or 3, most preferably 1 second segment (S₂).

When the monolithic pharmaceutical dosage form contains only one first segment (S₁) and only one second segment (S₂), the monolithic pharmaceutical dosage form is preferably a mantle tablet.

When the monolithic pharmaceutical dosage form contains more than one first segment (S₁) and/or more than one second segment (S₂), the monolithic pharmaceutical dosage form is preferably a layered tablet or a mantled layered tablet.

While the monolithic pharmaceutical dosage form may contain additional segments (S₃), e.g. segments which contain pharmacologically active ingredient but are essentially not of the same type and nature as first segments (S₁) and second segments (S₂), respectively, the monolithic pharmaceutical dosage form preferably does not contain additional segments (S₃).

For the purpose of specification, a coating such as e.g. a film coating preferably does not contain any pharmacologically active ingredient and does not constitute any segment of the monolithic pharmaceutical dosage form.

In a preferred embodiment, the monolithic pharmaceutical dosage form consists of
(i) at least one first segment (S₁);
(ii) at least one second segment (S₂); and
(iii) optionally a film coating.

In a particularly preferred embodiment, the monolithic pharmaceutical dosage form consists of
(i) at least one first segment (S₁) containing a first pharmacologically active ingredient (A₁);
(ii) at least one second segment (S₂) containing a second pharmacologically active ingredient (A₂); and
(iii) optionally a film coating.

According to this preferred embodiment, the monolithic pharmaceutical dosage form as such is preferably hot melt-extruded and optionally subsequently applied with a film coating. Nevertheless, it is principally also possible that the at least one hot melt-extruded first segment (S₁) preferably containing a first pharmacologically active ingredient (A₁) and the at least one preferably hot melt-extruded second segment (S₂) preferably containing a second pharmacologically active ingredient (A₂) are compacted with one another by another thermoforming process yielding the monolithic pharmaceutical dosage form that is optionally subsequently applied with a film coating.

In a preferred embodiment, the hot melt-extruded first segment(s) (S₁) and the preferably hot melt-extruded second segment(s) (S₂) each constitute a spatially confined area within the pharmaceutical dosage form. According to this embodiment, the first segment (S₁) and/or second segment (S₂) preferably forms a layer, a core or a mantle of the monolithic pharmaceutical dosage form which is preferably in form of a tablet.

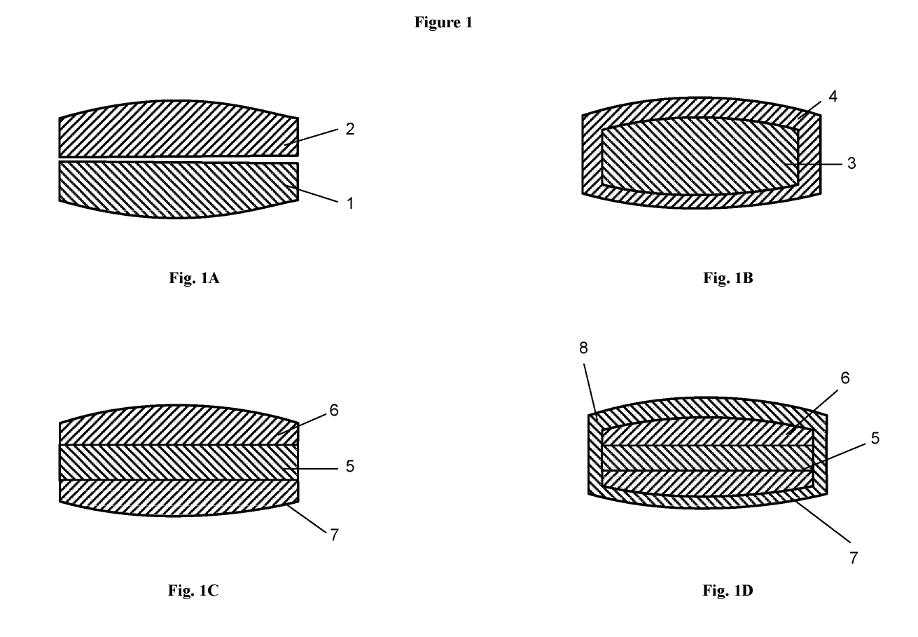

Preferred embodiments of tablets comprising the first segment (S₁) and the second segment (S₂) are illustrated in FIG. 1.

FIG. 1A schematically illustrates a two-layer tablet comprising a first segment (S₁) as first layer (1) and a second segment (S₂) as second layer (2).

FIG. 1B schematically illustrates a mantle tablet comprising a first segment (S₁) as a core (3) and a second segment (S₂) (4) surrounding said core (3).

FIG. 1C schematically illustrates a three-layer tablet comprising a first segment (S₁) as first layer (5) and two second segments (S₂) as layer (6) and layer (7).

FIG. 1D schematically illustrates a mantled three-layer tablet comprising a first segment (S₁) as first layer (5) and mantle (8) and two second segments (S₂) as layer (6) and layer (7).

Preferably, the content of the first segment(s) (S₁) in the monolithic pharmaceutical dosage form according to the invention is at most 99 wt.-%, more preferably at most 95 wt.-%, still more preferably at most 90 wt.-%, yet more preferably at most 85 wt.-%, most preferably at most 82 wt.-% and in particular at most 80 wt.-%, based on the total weight of the first segment(s) (S₁) and on the total weight of the monolithic pharmaceutical dosage form. In a particularly preferred embodiment, the content of the first segment(s) (S₁) in the monolithic pharmaceutical dosage form according to the invention is at most 75 wt.-%, more preferably at most 70 wt.-%, still more preferably at most 65 wt.-%, yet more preferably at most 60 wt.-%, most preferably at most 55 wt.-% and in particular at most 50 wt.-%, based on the total weight of the first segment(s) (S₁) and on the total weight of the monolithic pharmaceutical dosage form.

Particularly preferably, the content of the first segment(s) (S₁) in the monolithic pharmaceutical dosage form according to the invention is at least 1 wt.-%, more preferably at least 5 wt.-%, still more preferably at least 10 wt.-%, even more preferably at least 13 wt.-%, yet more preferably at least 15 wt.-%, most preferably at least 18 wt.-% and in particular at least 20 wt.-%; based on the total weight of the first segment(s) (S₁) and on the total weight of the monolithic pharmaceutical dosage form. In another preferred embodiment, the content of the first segment(s) (S₁) in the monolithic pharmaceutical dosage form according to the invention is at least 25 wt.-%, more preferably at least 30 wt.-%, still more preferably at least 35 wt.-%, even more preferably at least 40 wt.-%, yet more preferably at least 45 wt.-%, most preferably at least 48 wt.-% and in particular at least 50 wt.-%; based on the total weight of the first segment(s) (S₁) and on the total weight of the monolithic pharmaceutical dosage form.

In a particularly preferred embodiment, the content of the first segment(s) (S₁) in the monolithic pharmaceutical dosage form according to the invention is at least 15, more preferably at least 18 and most preferably at least 20 wt.-% and at most 60, more preferably at most 55 and most preferably at most 50 wt.-%, based on the total weight of the first segment(s) (S₁) and on the total weight of the monolithic pharmaceutical dosage form.

Particularly preferably, the content of the second segment(s) (S₂) in the monolithic pharmaceutical dosage form according to the invention is at most 99 wt.-%, more preferably at most 95 wt.-%, still more preferably at most 90 wt.-%, yet more preferably at most 85 wt.-%, most preferably at most 82 wt.-% and in particular at most 80 wt.-%, based on the total weight of the second segment(s) (S₂) and on the total weight of the monolithic pharmaceutical dosage form. In another preferred embodiment, the content of the second segment(s) (S₂) in the monolithic pharmaceutical dosage form according to the invention is at most 75 wt.-%, more preferably at most 70 wt.-%, still more preferably at most 65 wt.-%, yet more preferably at most 60 wt.-%, most preferably at most 55 wt.-% and in particular at most 50 wt.-%, based on the total weight of the second segment(s) (S₂) and on the total weight of the monolithic pharmaceutical dosage form.

Preferably, the content of the second segment(s) (S₂) in the monolithic pharmaceutical dosage form according to the invention is at least 1 wt.-%, more preferably at least 5 wt.-%, still more preferably at least 10 wt.-%, even more preferably at least 13 wt.-%, yet more preferably at least 15 wt.-%, most preferably at least 18 wt.-% and in particular at least 20 wt.-%; based on the total weight of the second segment(s) (S₂) and on the total weight of the monolithic pharmaceutical dosage form. In particularly preferred embodiment, the content of the second segment(s) (S₂) in the monolithic pharmaceutical dosage form according to the invention is at least 25 wt.-%, more preferably at least 30 wt.-%, still more preferably at least 35 wt.-%, even more preferably at least 40 wt.-%, yet more preferably at least 45 wt.-%, most preferably at least 48 wt.-% and in particular at least 50 wt.-%; based on the total weight of the second segment(s) (S₂) and on the total weight of the monolithic pharmaceutical dosage form.

In particularly preferred embodiment, the content of the second segment(s) (S₂) in the monolithic pharmaceutical dosage form according to the invention is at least 45, more preferably at least 48 and most preferably at least 50 wt.-% and at most 85, more preferably at most 82 and most preferably at most 80 wt.-%, based on the total weight of the second segment(s) (S₂) and on the total weight of the monolithic pharmaceutical dosage form.

Preferably, the relative weight ratio of the first segment (S₁) to the second segment (S₂) in the monolithic pharmaceutical dosage form is within the range of from 90:10 to 10:90, more preferably 80:20 to 13:87, still more preferably 70:30 to 15:85, even more preferably 60:40 to 17:83, most preferably 55:45 to 19:81 and in particular 50:50 to 20:80.

In a preferred embodiment, the relative weight ratio of the first segment (S₁) to the second segment (S₂) in the monolithic pharmaceutical dosage form is within the range of 9.0±8.5:1.0, more preferably 9.0±7.0:1.0, still more preferably 9.0±5.0:1.0, most preferably 9.0±3.0:1.0 and in particular 9.0±1.0:1.0.

In another preferred embodiment, the relative weight ratio of the first segment (S₁) to the second segment (S₂) in the monolithic pharmaceutical dosage form is within the range of 2.0±0.8:1.0, more preferably 2.0±0.6:1.0, still more preferably 2.0±0.4:1.0, most preferably 2.0±0.3:1.0 and in particular 2.0±0.2:1.0.

In still another preferred embodiment, the relative weight ratio of the first segment (S₁) to the second segment (S₂) in the monolithic pharmaceutical dosage form is within the range of 1.0±0.8:1.0, more preferably 1.0±0.6:1.0, still more preferably 1.0±0.4:1.0, most preferably 1.0±0.3:1.0 and in particular 1.0±0.2:1.0.

In yet another preferred embodiment, the relative weight ratio of the first segment (S₁) to the second segment (S₂) in the monolithic pharmaceutical dosage form is within the range of 1.0:1.0±0.8, more preferably 1.0:1.0±0.6, still more preferably 1.0:1.0±0.4, most preferably 1.0:1.0±0.3 and in particular 1.0:1.0±0.2.

In a further preferred embodiment, the relative weight ratio of the first segment (S₁) to the second segment (S₂) in the monolithic pharmaceutical dosage form is within the range of 1.0:2.0±0.8, more preferably 1.0:2.0±0.6, still more preferably 1.0:2.0±0.4, most preferably 1.0:2.0±0.3 and in particular 1.0:2.0±0.2.

In still a further preferred embodiment, the relative weight ratio of the first segment (S₁) to the second segment (S₂) in the monolithic pharmaceutical dosage form is within the range of 1.0:9.0±8.5, more preferably 1.0:9.0±7.0, still more preferably 1.0:9.0±5.0, most preferably 1.0:9.0±3.0 and in particular 1.0:9.0±1.0.

The shape of the segments, i.e. the shape of the first segment(s) (S₁) and/or the second segment(s) (S₂), is not particularly limited. When a segment forms a layer, e.g. in a layered tablet, it preferably has a sheet-like structure. When a segment forms a tablet core, e.g. in a mantle tablet, it preferably is essentially spherical and more preferably essentially cylindrical in shape, e.g. cut extruded rods. The diameter of such an essentially cylindrical segment is therefore the diameter of its circular cross section. The cylindrical shape is preferably caused by hot melt extrusion according to which the diameter of the circular cross section is a function of the extrusion die and the length of the cylinders is a function of the cutting length according to which the extruded strand of material is cut into pieces of preferably more or less predetermined length. When a segment forms a mantle, e.g. in a mantle tablet or a mantled layered tablet, it preferably has the shape of a hollow cylinder, more preferably a hollow sphere and most preferably a hollow ellipsoid.

In a preferred embodiment, neither the first segment(s) (S₁) nor the second segment(s) (S₂) are provided with a coating.

In another preferred embodiment, the first segment(s) (S₁) and/or the second segment(s) (S₂) are coated, more preferably film coated. According to this embodiment, preferably only the second segment (S₂) is film coated, wherein the second segment (S₂) covers the entire surface of the first segment (S₁).

The first segment(s) (S₁) and/or the second segment(s) (S₂) according to the invention can optionally be provided, partially or completely, with a coating, preferably a film coating. When the first segment(s) (S₁) and the second segment(s) (S₂) are each partially provided with a coating, they are preferably arranged in immediately adjacent layers forming a layered structure wherein said layered structure is preferably provided with a coating.

When the first segment(s) (S₁) and/or the second segment(s) (S₂) are provided with a coating, conventional film coating compositions are preferred. Suitable coating materials are commercially available, e.g. under the trademarks Opadry® and Eudragit®.

Examples of suitable materials include cellulose esters and cellulose ethers, such as methylcellulose (MC), hydroxypropylmethylcellulose (HPMC), hydroxypropylcellulose (HPC), hydroxyethylcellulose (HEC), sodium carboxymethylcellulose (Na-CMC), ethylcellulose (EC), cellulose acetate phthalate (CAP), hydroxypropylmethylcellulose phthalate (HPMCP); poly(meth)acrylates, such as aminoalkylmethacrylate copolymers, ethylacrylate methylmethacrylate copolymers, methacrylic acid methylmethacrylate copolymers, methacrylic acid methylmethacrylate copolymers; vinyl polymers, such as polyvinylpyrrolidone, polyvinylacetatephthalate, polyvinyl alcohol, polyvinyl alcohol-polyethylene glycol graft copolymers, polyvinylacetate; and natural film formers.

The coating material may contain excipients such as stabilizers (e.g. surfactants such as macrogol cetostearylether, sodium dodecylsulfate, and the like). Suitable excipients of film coating materials are known to the skilled person.

In a particularly preferred embodiment, the coating is water-soluble.

Though less preferred, the coating can principally be resistant to gastric juices and dissolve as a function of the pH value of the release environment. By means of this coating, it is possible to ensure that the monolithic pharmaceutical dosage form according to the invention passes through the stomach undissolved and the active compound is only released in the intestines. The coating which is resistant to gastric juices preferably dissolves at a pH value of between 5 and 7.5. Corresponding materials and methods for the delayed release of active compounds and for the application of coatings which are resistant to gastric juices are known to the person skilled in the art, for example from “Coated Pharmaceutical dosage forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers.

A particularly preferred coating contains polyvinyl alcohol and optionally, further excipients such as xanthan gum and/or talcum.

For the purpose of specification, the term “pharmacologically active ingredient” as used herein may refer to either one or more pharmacologically active ingredients, i.e. the terms “first pharmacologically ingredient (A₁)”, “second pharmacologically ingredient (A₂)” and “further pharmacologically ingredient (A_{f})” may each refer to a single pharmacologically active ingredient or a combination of one or more pharmacologically active ingredients.

There are generally no limitations as to the pharmacologically active ingredient (pharmacologically active compound) which can be incorporated in the segments of the monolithic pharmaceutical dosage form according to the invention. Furthermore, the term “pharmacologically active ingredient” preferably includes any physiologically acceptable salt, e.g. physiologically acceptable acid addition salt, of the base form of the pharmacologically active ingredient. Physiologically acceptable acid addition salts comprise any acid addition salts which can conveniently be obtained by treating the base form of a pharmacologically active ingredient with appropriate organic and inorganic acids. Pharmacologically active ingredients containing an acidic proton may be converted into their non-toxic metal or amine addition salt forms by treatment with appropriate organic and inorganic bases. The term addition salt also comprises the hydrates and solvent addition forms which a pharmacologically active ingredient is able to form. Examples of such forms are e.g. hydrates, alcoholates and the like.

Unless explicitly stated otherwise, all amounts of the first pharmacologically active ingredient (A₁), the second pharmacologically active ingredient (A₂) and the further pharmacologically active ingredient (A_{f}) specified in the following are given according to the corresponding amount of the free compound.

In a preferred embodiment, the first pharmacologically active ingredient (A₁) is an opioid and the second pharmacologically active ingredient (A₂) is another analgesic, but preferably no opioid, e.g. an NSAID or COX-2-inhibitor.

In another preferred embodiment, the first pharmacologically active ingredient (A₁) and the second pharmacologically active ingredient (A₂), respectively, is an opioid, wherein the first pharmacologically active ingredient (A₁) is equal to or is different from the second pharmacologically active ingredient (A₂).

In still another preferred embodiment, the first pharmacologically active ingredient (A₁) is an analgesic, but preferably no opioid, e.g. an NSAID or COX-2-inhibitor, and the second pharmacologically active ingredient (A₂) is an opioid.

In a further preferred embodiment, the first segment (S₁) contains a first pharmacologically active ingredient (A₁) and a further pharmacologically active ingredient (A_{f}), whereas the second segment (S₂) does not contain any pharmacologically active ingredient.

According to this embodiment, preferably, the first pharmacologically active ingredient (A₁) is an opioid and the further pharmacologically active ingredient (A_{f}) is another analgesic, but preferably no opioid, e.g. an NSAID or COX-2-inhibitor.

Further according to this embodiment, preferably, the first pharmacologically active ingredient (A₁) and the further pharmacologically active ingredient (A_{f}), respectively, is an opioid, wherein the first pharmacologically active ingredient (A₁) is different from the further pharmacologically active ingredient (A_{f}).

Still further according to this embodiment, preferably, the first pharmacologically active ingredient (A₁) is an analgesic, but preferably no opioid, e.g. an NSAID or COX-2-inhibitor, and the further pharmacologically active ingredient (A_{f}) is an opioid.

In another preferred embodiment, the second segment (S₂) contains a second pharmacologically active ingredient (A₂) and a further pharmacologically active ingredient (A_{f}), whereas the first segment (S₁) does not contain any pharmacologically active ingredient.

According to this embodiment, preferably, the second pharmacologically active ingredient (A₂) is an opioid and the further pharmacologically active ingredient (A_{f}) is another analgesic, but preferably no opioid, e.g. an NSAID or COX-2-inhibitor.

Further according to this embodiment, preferably, the second pharmacologically active ingredient (A₂) and the further pharmacologically active ingredient (A_{f}), respectively, is an opioid, wherein the second pharmacologically active ingredient (A₂) is different from the further pharmacologically active ingredient (A_{f}).

Still further according to this embodiment, preferably, the second pharmacologically active ingredient (A₂) is an analgesic, but preferably no opioid, e.g. an NSAID or COX-2-inhibitor, and the further pharmacologically active ingredient (A_{f}) is an opioid.

In a preferred embodiment, the first pharmacologically active ingredient (A₁) and the second pharmacologically active ingredient (A₂) are spatially separated from one another. According to this embodiment, the first segment (S₁) preferably contains less than 0.1 ppm, more preferably less than 0.01 ppm, most preferably less than 0.001 ppm and in particular less than 0.0001 ppm of the second pharmacologically active ingredient (A₂). Further, according to this embodiment, the second segment (S₂) preferably contains less than 0.1 ppm, more preferably less than 0.01 ppm, most preferably less than 0.001 ppm and in particular less than 0.0001 ppm of the first pharmacologically active ingredient (A₁). In a particularly preferred embodiment, the first segment (S₁) contains no second pharmacologically active ingredient (A₂) and the second segment (S₂) contains no first pharmacologically active ingredient (A₁).

Preferably, at least 99 wt.-%, more preferably at least 99.9 wt.-%, most preferably at least 99.99 wt.-% and in particular at least 99.999 wt.-% of the total amount of the first pharmacologically active ingredient (A₁) contained in the monolithic pharmaceutical dosage form are contained in the first segment (S₁).

Preferably, at least 99 wt.-%, more preferably at least 99.9 wt.-%, most preferably at least 99.99 wt.-% and in particular at least 99.999 wt.-% of the total amount of the second pharmacologically active ingredient (A₂) contained in the monolithic pharmaceutical dosage form are contained in the second segment (S₂).

In another preferred embodiment, the first pharmacologically active ingredient (A₁) contained in the first segment (S₁) and the second pharmacologically active ingredient (A₂) contained in the second segment (S₂) are identical.

The term “prolonged release” is known to the skilled artisan. For the purpose of specification, the term “prolonged release” preferably refers to a release rate of the pharmacologically active ingredient from the formulation that has been reduced over time in order to maintain therapeutic activity, to reduce toxic effects, or for some other therapeutic purpose such as reducing the dosing frequency.

The term “immediate release” is known to the skilled artisan. For the purpose of specification, the term “immediate release” preferably refers to a release rate of the pharmacologically active ingredient from the formulation that is comparatively fast and not retarded.

In the monolithic pharmaceutical dosage form according to the present invention, the release of the first pharmacologically active ingredient (A₁) and the second pharmacologically active ingredient (A₂), respectively, is preferably neither controlled by erosion of the surface of the segment (S₁) and the segment (S₂), respectively, nor by erosion of the surface of the monolithic pharmaceutical dosage form.

In a further preferred embodiment, the first segment (S₁) and/or the second segment (S₂) constitute a spatially confined area within the pharmaceutical dosage form. According to this embodiment, the first segment (S₁) and/or second segment (S₂) preferably form a layer, a core or a mantle of the monolithic pharmaceutical dosage form.

In a preferred embodiment, the monolithic pharmaceutical dosage form is a mantle tablet.

According to the present invention, the term “mantle tablet” preferably includes tablets in which one segment covers the entire surface of the other segment forming the tablet core, as well as tablets in which one segment preferably covers at least 75%, more preferably at least 80%, still more preferably at least 85%, yet more preferably at least 90%, most preferably at least 95% and in particular at least 99% of the surface of the other segment forming the tablet core.

In another preferred embodiment, the term “mantle tablet” includes tablets in which one segment covers the tablet core, wherein the tablet core has a layered structure with every layer constituting a segment. According to this embodiment, the monolithic pharmaceutical dosage form is preferably a mantled layered tablet, which is described in more detail further below.

However, in a particularly preferred embodiment, when the monolithic pharmaceutical dosage form is a mantle tablet, the tablet core constitutes a single segment and, thus, has no layered structure.

Preferably, when the monolithic pharmaceutical dosage form is provided in form of a mantle tablet, the tablet core constitutes one segment whereas the mantle (also known as shell) constitutes another segment of the dosage form. The mantle tablet, more preferably the mantle of the mantle tablet, may optionally be provided with a film coating.

When the monolithic pharmaceutical dosage form is provided in form of a mantle tablet, it may also comprise more than one, i.e. two or three mantles. Particularly preferably, however, when the monolithic pharmaceutical dosage form is provided in form of a mantle tablet, it comprises only one core and only one mantle.

A mantle of a mantle tablet is to be distinguished from a coating. According to the present invention, a mantle is preferably hot melt-extruded whereas a coating is not hot melt-extruded but is applied to a dosage form as a suspension or a solution by spray-coating (e.g. in a coating pan or a fluidized bed coater) or as a solid (e.g. by compression coating or as a powder coating).

In a preferred embodiment, the monolithic pharmaceutical dosage form is a mantle tablet, wherein the first segment (S₁) preferably forms the tablet core and the second segment (S₂) preferably forms the mantle (cf. FIG. 1B). According to this embodiment, the second segment (S₂) preferably covers the entire surface of the first segment (S₁).

Preferably, the relative weight ratio of the first segment (S₁) preferably forming the tablet core to the second segment (S₂) preferably forming the mantle is within the range of from 90:10 to 10:90, more preferably 80:20 to 13:87, still more preferably 70:30 to 15:85, even more preferably 60:40 to 17:83, most preferably 55:45 to 19:81 and in particular 50:50 to 20:80.

In another preferred embodiment, the monolithic pharmaceutical dosage form is a layered tablet. According to this embodiment, the first segment(s) (S₁) and/or the second segment(s) (S₂) form a layer (cf. FIG. 1A and FIG. 1C).

When the monolithic pharmaceutical dosage form is provided in form of a layered tablet, every layer of the layered tablet constitutes a segment of the monolithic dosage form. The layered tablet may optionally be provided with a film coating. However, the individual layers of the layered tablet are preferably not provided with a film coating.

When the monolithic pharmaceutical dosage form is provided in form of a layered tablet, any layer of the first segment (S₁) preferably is directly adjacent to a layer of the second segment (S₂). Preferred layer sequences of a layered tablet include but are not limited to (S₂)/(S₁), (S₂)/(S₁)/(S₂), (S₁)/(S₂)/(S₁) or (S₁)/(S₂)/(S₁)/(S₂). Layered tablets having two or three layers are particularly preferred.

Preferably, the relative weight ratio of the combined layers formed by the first segment (S₁) to the combined layers formed by the second segment (S₂) is within the range of from 90:10 to 10:90, more preferably 80:20 to 13:87, still more preferably 70:30 to 15:85, even more preferably 60:40 to 17:83, most preferably 55:45 to 19:81 and in particular 50:50 to 20:80.

In another preferred embodiment, the monolithic pharmaceutical dosage form is a mantled layered tablet.

For the purpose of specification a mantled layered tablet refers to a tablet having a layered inner structure wherein this layered inner structure is enclosed by a mantle (cf. FIG. 1 D). The mantle enclosing the layered inner structure may cover the entire surface of the layered inner structure or may cover at least 75%, preferably at least 80%, more preferably at least 85%, still more preferably at least 90%, most preferably at least 95% and in particular at least 99% of the surface of the layered inner structure.

When the monolithic pharmaceutical dosage form is provided in form of a mantled layered tablet, the mantle and every layer of the layered tablet constitute a segment of the dosage form. The mantled layered tablet, preferably the mantle of the mantled layered tablet, may optionally be provided with a film coating.

When the monolithic pharmaceutical dosage form is provided as a mantled layered tablet, preferred layer sequences of the layered inner structure include but are not limited to (S₂)/(S₁), (S₂)/(S₁)/(S₂), (S₁)/(S₂)/(S₁) or (S₁)/(S₂)/(S₁)/(S₂). Layered inner structures having two or three layers are particularly preferred. The mantle of a mantled layered tablet may be formed by the first segment (S₁) or the second segment (S₂).

Preferably, when the monolithic pharmaceutical dosage form is provided as a mantled layered tablet, the relative weight ratio of the total amount of the first segment (S₁) to the total amount of the second segment (S₂) is within the range of from 90:10 to 10:90, more preferably 80:20 to 13:87, still more preferably 70:30 to 15:85, even more preferably 60:40 to 17:83, most preferably 55:45 to 19:81 and in particular 50:50 to 20:80.

In a preferred embodiment, the monolithic pharmaceutical dosage form is a tablet with armoring layer comprising a tablet core and an armoring layer.

According to the present invention, the term “armoring layer” preferably relates to an entity which is not brittle, hard to cut and preferably has a high breaking strength of at least 300 N, more preferably at least 400 N and most preferably at least 500 N. Furthermore, the armoring layer is firmly attached to the tablet core so that preferably the armoring layer cannot be separated from the tablet core by conventional means available to an abuser, i.e. such as cutting with a knife or striking with a hammer.

The armoring layer can be hot melt extruded or not hot melt extruded. When the armoring layer is not hot melt extruded, it is preferably applied to the tablet core e.g. as a suspension or a solution by spray-coating (e.g. in a coating pan or a fluidized bed coater) or as a solid (e.g. by compression coating or as a powder coating). Preferably, the armoring layer has a thickness of at least 200 μm, more preferably at least 300 μm, still more preferably at least 400 μm, yet more preferably at least 500 μm, even more preferably at least 600 μm, most preferably at least 700 μm or at least 800 μm and in particular at least 900 μm, at least 1,000 μm or at least 1,500 μm.

Preferably, the armoring layer covers the entire surface of the other segment forming the tablet core. In another preferred embodiment, the armoring layer covers at least 75%, more preferably at least 80%, still more preferably at least 85%, yet more preferably at least 90%, most preferably at least 95% and in particular at least 99% of the surface of the tablet core.

In a particularly preferred embodiment, when the monolithic pharmaceutical dosage form is a tablet with armoring layer, the tablet core has no layered structure, thus, constituting one single segment.

Preferably, when the monolithic pharmaceutical dosage form is provided in form of a tablet with armoring layer, the tablet core constitutes one segment whereas the armoring layer constitutes another segment of the dosage form. The tablet with armoring layer, more preferably the armoring layer, may optionally be provided with a film coating.

When the monolithic pharmaceutical dosage form is provided in form of a tablet with armoring layer, it may also comprise more than one, i.e. two or three armoring layers. Particularly preferably, however, when the monolithic pharmaceutical dosage form is provided in form of a tablet with armoring layer, it comprises only one core and only one armoring layer.

In a preferred embodiment, the monolithic pharmaceutical dosage form is a tablet with armoring layer, wherein the first segment (S₁) preferably forms the tablet core and the second segment (S₂) preferably forms the armoring layer. According to this embodiment, the second segment (S₂) preferably covers the entire surface of the first segment (S₁).

Preferably, the relative weight ratio of the first segment (S₁) preferably forming the tablet core to the second segment (S₂) preferably forming the armoring layer is within the range of from 90:10 to 10:90, more preferably 80:20 to 13:87, still more preferably 70:30 to 15:85, even more preferably 60:40 to 17:83, most preferably 55:45 to 19:81 and in particular 50:50 to 20:80.

In a preferred embodiment, the monolithic pharmaceutical dosage form according to the invention is a tablet. According to this embodiment, the tablet preferably comprises
(i) a co-extrudate of one or more first segment(s) (S₁) and one or more second segment(s) (S₂) that are arranged in a seamless manner in form of a layered structure, wherein the layers can be parallel or concentric to one another; and/or
(ii) a single first segment (S₁) and a single second segment (S₂) that are arranged to form a bilayer tablet (cf. FIG. 1A);
(iii) a single first segment (S₁) forming a core that is surrounded by a single second segment (S₂) such that first segment (S₁) and second segment (S₂) are arranged to form a mantle tablet (cf. FIG. 1B);
(iv) a single first segment (S₁) and two second segments (S₂) that are arranged to form a trilayer tablet, wherein first segment (S₁) forms the middle layer and the two second segments (S₂) form the outer layers (cf. FIG. 1C);
(v) a plurality of first segments (S₁) and a plurality of second segments (S₂) that are arranged to form a layered tablet, wherein preferably each of the first segments (S₁) is arranged in between two adjacent second segments (S₂); or
(vi) a single first segment (S₁) and two second segments (S₂) that are arranged to form a trilayer structure, wherein the first segment (S₁) forms the middle layer and the two second segments (S₂) form the outer layers and wherein said trilayer structure is provided with a mantle formed by a further first segment (S₁) (cf. FIG. 1D); or a single second segment (S₂) and two first segments (S₁) that are arranged to form a trilayer structure, wherein the second segment (S₂) forms the middle layer and the two first segments (S₁) form the outer layers and wherein said trilayer structure is provided with a mantle formed by a further second segment (S₂).

The monolithic pharmaceutical dosage form comprises a first segment (S₁), which preferably contains a first pharmacologically active ingredient (A₁).

In another preferred embodiment, the monolithic pharmaceutical dosage form comprises a first segment (S₁), which does not contain any pharmacologically active ingredient.

In a preferred embodiment, the segment (S₁) provides prolonged release of the first pharmacologically active ingredient (A₁). In another preferred embodiment, the segment (S₁) provides immediate release of the first pharmacologically active ingredient (A₁).

In a preferred embodiment, the first pharmacologically active ingredient (A₁) is only a single pharmacologically active ingredient. In another preferred embodiment, the first pharmacologically active ingredient (A₁) is a combination of two or more pharmacologically active ingredients.

Preferably, the first pharmacologically active ingredient (A₁) has potential for being abused. Pharmacologically active ingredients with potential for being abused are known to the person skilled in the art and comprise e.g. tranquillizers, stimulants, barbiturates, narcotics, opioids or opioid derivatives.

Preferably, the first pharmacologically active ingredient (A₁) has a psychotropic effect, i.e. crosses the blood-brain barrier and acts primarily upon the central nervous system where it affects brain function, resulting in alterations in perception, mood, consciousness, cognition, and behavior.

Preferably, the first pharmacologically active ingredient (A₁) is selected from the group consisting of opioids, stimulants, tranquilizers, and other narcotics.

Particularly preferably, the first pharmacologically active ingredient (A₁) is an opioid or a physiologically acceptable salt thereof. According to the Anatomical Therapeutic Chemical (ATC) classification system by WHO (ATC index), opioids are divided into natural opium alkaloids, phenylpiperidine derivatives, diphenylpropylamine derivatives, benzomorphan derivatives, oripavine derivatives, morphinan derivatives and others. Preferably, the second pharmacologically active ingredient (A₂) is selected from ATC classes [M01A], [M01d], [N02B] and [N02C] according to the WHO.

The following opioids, tranquillizers or other narcotics are substances with a psychotropic action, i.e. have a potential of abuse, and hence are preferably contained in the first segment (S₁) of the monolithic pharmaceutical dosage form according to the invention: alfentanil, allobarbital, allylprodine, alphaprodine, alprazolam, amfepramone, amphetamine, amphetaminil, amobarbital, anileridine, apocodeine, axomadol, barbital, bemidone, benzylmorphine, bezitramide, bromazepam, brotizolam, buprenorphine, butobarbital, butorphanol, camazepam, carfentanil, cathine/D-norpseudoephedrine, chlordiazepoxide, clobazam clofedanol, clonazepam, clonitazene, clorazepate, clotiazepam, cloxazolam, cocaine, codeine, cyclobarbital, cyclorphan, cyprenorphine, delorazepam, desomorphine, dextromoramide, dextropropoxyphene, dezocine, diampromide, diamorphone, diazepam, dihydrocodeine, dihydromorphine, dihydromorphone, dimenoxadol, dimephetamol, dimethylthiambutene, dioxaphetylbutyrate, dipipanone, dronabinol, eptazocine, estazolam, ethoheptazine, ethylmethylthiambutene, ethyl loflazepate, ethylmorphine, etonitazene, etorphine, faxeladol, fencamfamine, fenethylline, fenpipramide, fenproporex, fentanyl, fludiazepam, flunitrazepam, flurazepam, halazepam, haloxazolam, heroin, hydrocodone, hydromorphone, hydroxypethidine, isomethadone, hydroxymethylmorphinan, ketazolam, ketobemidone, levacetylmethadol (LAAM), levomethadone, levorphanol, levophenacyl-morphane, levoxemacin, lisdexamfetamine dimesylate, lofentanil, loprazolam, lorazepam, lormetazepam, mazindol, medazepam, mefenorex, meperidine, meprobamate, metapon, meptazinol, metazocine, methylmorphine, metamphetamine, methadone, methaqualone, 3-methylfentanyl, 4-methylfentanyl, methylphenidate, methylphenobarbital, methyprylon, metopon, midazolam, modafinil, morphine, myrophine, nabilone, nalbuphene, nalorphine, narceine, nicomorphine, nimetazepam, nitrazepam, nordazepam, norlevorphanol, normethadone, normorphine, norpipanone, opium, oxazepam, oxazolam, oxycodone, oxymorphone, Papaver somniferum, papaveretum, pemoline, pentazocine, pentobarbital, pethidine, phenadoxone, phenomorphane, phenazocine, phenoperidine, piminodine, pholcodeine, phenmetrazine, phenobarbital, phentermine, pinazepam, pipradrol, piritramide, prazepam, profadol, proheptazine, promedol, properidine, propoxyphene, remifentanil, secbutabarbital, secobarbital, sufentanil, tapentadol, temazepam, tetrazepam, tilidine (cis and trans), tramadol, triazolam, vinylbital, N-(1-methyl-2-piperidinoethyl)-N-(2-pyridyl)-propionamide, (1R,2R)-3-(3-dimethylamino-1-ethyl-2-methyl-propyl)phenol, (1R,2R,4 S)-2-(dimethylamino)methyl-4-(p-fluorobenzyloxy)-1-(m-methoxyphenyl)cyclohexanol, (1R,2R)-3-(2-dimethylaminomethyl-cyclohexyl)phenol, (1S,2S)-3-(3-dimethylamino-1-ethyl-2-methyl-propyl)phenol, (2R,3R)-1-dimethylamino-3(3-methoxyphenyl)-2-methyl-pentan-3-ol, (1RS,3RS,6RS)-6-dimethylaminomethyl-1-(3-methoxyphenyl)-cyclohexane-1,3-diol, preferably as racemate, 3-(2-dimethylaminomethyl-1-hydroxy-cyclohexyl)phenyl2-(4-isobutylphenyl)propionate, 3-(2-dimethylaminomethyl-1-hydroxy-cyclohexyl)phenyl2-(6-methoxy-naphthalen-2-yl)propionate, 3-(2-dimethylaminomethyl-cyclohex-1-enyl)-phenyl2-(4-isobutyl-phenyl)propionate, 3-(2-dimethylaminomethyl-cyclohex-1-enyl)-phenyl2-(6-methoxy-naphthalen-2-yl)propionate, (RR—SS)-2-acetoxy-4-trifluoromethyl-benzoic acid 3-(2-dimethylaminomethyl-1-hydroxy-cyclohexyl)-phenyl ester, (RR—SS)-2-hydroxy-4-trifluoromethyl-benzoic acid 3-(2-dimethylaminomethyl-1-hydroxy-cyclohexyl)-phenyl ester, (RR—SS)-4-chloro-2-hydroxy-benzoic acid 3-(2-dimethylaminomethyl-1-hydroxy-cyclohexyl)-phenyl ester, (RR—SS)-2-hydroxy-4-methyl-benzoic acid 3-(2-dimethylaminomethyl-1-hydroxy-cyclohexyl)-phenyl ester, (RR—SS)-2-hydroxy-4-methoxybenzoic acid 3-(2-dimethylaminomethyl-1-hydroxy-cyclohexyl)-phenyl ester, (RR—SS)-2-hydroxy-5-nitro-benzoic acid 3-(2-dimethylaminomethyl-1-hydroxy-cyclohexyl)-phenyl ester, (RR—SS)-2′,4′-difluoro-3-hydroxy-biphenyl-4-carboxylic acid 3-(2-dimethylaminomethyl-1-hydroxy-cyclohexyl)-phenyl ester, and corresponding stereoisomeric compounds, in each case the corresponding derivatives thereof, physiologically acceptable enantiomers, stereoisomers, diastereomers and racemates and the physiologically acceptable derivatives thereof, e.g. ethers, esters or amides, and in each case the physiologically acceptable compounds thereof, in particular the acid or base addition salts thereof and solvates, e.g. hydrochlorides.

In a preferred embodiment, the first segment (S₁) contains an opioid selected from the group consisting of DPI-125, M6G (CE-04-410), ADL-5859, CR-665, NRP290 and sebacoyl dinalbuphine ester.

In a preferred embodiment, the first segment (S₁) contains the first pharmacologically active ingredient (A₁) which is one pharmacologically active ingredient or more pharmacologically active ingredients selected from the group consisting of tramadol, oxycodone, oxymorphone, hydromorphone, hydrocodone, morphine, buprenorphine and tapentadol and the physiologically acceptable salts thereof.

In another preferred embodiment, the first pharmacologically active ingredient (A₁) is selected from the group consisting of tapentadol, faxeladol, axomadol and the physiologically acceptable salts thereof.

In still another preferred embodiment, the first pharmacologically active ingredient (A₁) is selected from the group consisting of 1,1-(3-dimethylamino-3-phenylpentamethylene)-6-fluoro-1,3,4,9-tetrahydropyrano[3,4-b]indole (cebranopadol), particularly its hemicitrate; 1,1-[3-dimethylamino-3-(2-thienyl)pentamethylene]-1,3,4,9-tetrahydropyrano[3,4-b]indole, particularly its citrate; and 1,1-[3-dimethylamino-3-(2-thienyl)pentamethylene]-1,3,4,9-tetrahydropyrano[3,4-b]-6-fluoroindole, particularly its hemicitrate. These compounds are known from, e.g., WO 2004/043967, WO 2005/066183.

In a particularly preferred embodiment, the first segment (S₁) provides prolonged release of the first pharmacologically active ingredient (A₁) which preferably is an opioid or a physiologically acceptable salt thereof.

In another preferred embodiment, the first pharmacologically active ingredient (A₁) exhibits no psychotropic action. In a preferred embodiment, when the first pharmacologically active ingredient (A₁) exhibits no psychotropic action, the first segment (S₁) provides immediate release of the first pharmacologically active ingredient (A₁). In another preferred embodiment, when the first pharmacologically active ingredient (A₁) exhibits no psychotropic action, the first segment (S₁) provides prolonged release of the first pharmacologically active ingredient (A₁).

In another preferred embodiment, the first pharmacologically active ingredient (A₁) is selected from ATC classes [M01A], [M01d], [N02B] and [N02C] according to the WHO.

Preferably, the first pharmacologically active ingredient (A₁) is selected from the group consisting of acetylsalicylic acid, aloxiprin, choline salicylate, sodium salicylate, salicylamide, salsalate, ethenzamide, morpholine salicylate, dipyrocetyl, benorilate, diflunisal, potassium salicylate, guacetisal, carbasalate calcium, imidazole salicylate, phenazone, metamizole sodium, aminophenazone, propyphenazone, nifenazone, paracetamol, phenacetin, bucetin, propacetamol, rimazolium, glafenine, floctafenine, viminol, nefopam, flupirtine, ziconotide, methoxyflurane, nabiximols, dihydroergotamine, ergotamine, methysergide, lisuride, flumedroxone, sumatriptan, naratriptan, zolmitriptan, rizatriptan, almotriptan, eletriptan, frovatriptan, pizotifen, clonidine, iprazochrome, dimetotiazine, oxetorone, phenylbutazone, mofebutazone, oxyphenbutazone, clofezone, kebuzone, indomethacin, sulindac, tolmetin, zomepirac, diclofenac, alclofenac, bumadizone, etodolac, lonazolac, fentiazac, acemetacin, difenpiramide, oxametacin, proglumetacin, ketorolac, aceclofenac, bufexamac, piroxicam, tenoxicam, droxicam, lornoxicam, meloxicam, ibuprofen, naproxen, ketoprofen, fenoprofen, fenbufen, benoxaprofen, suprofen, pirprofen, flurbiprofen, indoprofen, tiaprofenic acid, oxaprozin, ibuproxam, dexibuprofen, flunoxaprofen, alminoprofen, dexketoprofen, naproxcinod, mefenamic acid, tolfenamic acid, flufenamic acid, meclofenamic acid, celecoxib, rofecoxib, valdecoxib, parecoxib, etoricoxib, lumiracoxib, nabumetone, niflumic acid, azapropazone, glucosamine, benzydamine, glucosaminoglycan polysulfate, proquazone, orgotein, nimesulide, feprazone, diacerein, momiflumate, tenidap, oxaceprol, chondroitin sulfate, oxycinchophen, sodium aurothiomalate, sodium aurotiosulfate, auranofin, aurothioglucose, aurotioprol, penicillamine, bucillamine, their physiologically acceptable salts, as well as mixtures thereof.

Preferably, the first pharmacologically active ingredient (A₁) is present in the monolithic pharmaceutical dosage form in a therapeutically effective amount. In general, the amount that constitutes a therapeutically effective amount varies according to the pharmacologically active ingredients being used, the condition being treated, the severity of said condition, the patient being treated, and whether the monolithic pharmaceutical dosage form or the segment in which the pharmacologically active ingredient is contained is designed for an immediate or retarded release.

The content of the first pharmacologically active ingredient (A₁) preferably ranges from about 0.01 wt.-% to about 95 wt.-%, more preferably from about 0.1 wt.-% to about 90 wt.-%, even more preferably from about 0.3 wt.-% to about 85 wt.-%, yet more preferably from about 0.4 wt.-% to about 83 wt.-%, and most preferably from about 0.5 wt.-% to 82 wt.-%, based on the total weight of the first segment(s) (S₁) or based on the total weight of the monolithic pharmaceutical dosage form.

In a preferred embodiment, the content of the first pharmacologically active ingredient (A₁) is within the range of from 0.01 to 85 wt.-%, more preferably 0.1 to 60 wt.-%, still more preferably 0.3 to 40 wt.-%, most preferably 0.4 to 25 wt.-% and in particular 0.5 to 15 wt.-%, based on the total weight of the monolithic pharmaceutical dosage form. In another preferred embodiment, the content of the first pharmacologically active ingredient (A₁) is within the range of from 1 to 95 wt.-%, more preferably 3 to 80 wt.-%, still more preferably 5 to 70 wt.-%, most preferably 7 to 60 wt.-% and in particular 8 to 50 wt.-%, based on the total weight of the monolithic pharmaceutical dosage form.

In a preferred embodiment, the content of the first pharmacologically active ingredient (A₁) is within the range of from 1±0.9 wt.-%, 5±4 wt.-% or 7±6 wt.-%, more preferably 1±0.8 wt.-%, 5±3.5 wt.-% or 7±5 wt.-%, still more preferably 1±0.6 wt.-%, 5±3.0 wt.-% or 7±4 wt.-%, most preferably 1±0.4 wt.-%, 5±2.5 wt.-% or 7±3 wt.-%, and in particular 1±0.2 wt.-%, 5±2 wt.-% or 7±2 wt.-%, based on the total weight of the monolithic pharmaceutical dosage form. In another preferred embodiment, the content of the first pharmacologically active ingredient (A₁) is within the range of from 9±8 wt.-%, 12±11 wt.-% or 17±15 wt.-%, more preferably 9±6 wt.-%, 12±8 wt.-% or 17±12 wt.-%, still more preferably 9±4 wt.-%, 12±6 wt.-% or 17±9 wt.-%, most preferably 9±3 wt.-%, 12±4 wt.-% or 17±5 wt.-%, and in particular 9±2 wt.-%, 12±2 wt.-% or 17±2 wt.-%, based on the total weight of the monolithic pharmaceutical dosage form. In another preferred embodiment, the content of the first pharmacologically active ingredient (A₁) is within the range of from 20±18 wt.-%, 25±20 wt.-% or 30±25 wt.-%, more preferably 20±12 wt.-%, 25±15 wt.-% or 30±18 wt.-%, still more preferably 20±9 wt.-%, 25±10 wt.-% or 30±12 wt.-%, most preferably 20±6 wt.-%, 25±5 wt.-% or 30±7 wt.-%, and in particular 20±3 wt.-%, 25±3 wt.-% or 30±5 wt.-%, based on the total weight of the monolithic pharmaceutical dosage form. In another preferred embodiment, the content of the first pharmacologically active ingredient (A₁) is within the range of from 35±25 wt.-%, 40±25 wt.-% or 47±25 wt.-%, more preferably 35±18 wt.-%, 40±18 wt.-% or 47±18 wt.-%, still more preferably 35±12 wt.-%, 40±12 wt.-% or 47±12 wt.-%, most preferably 35±7 wt.-%, 40±7 wt.-% or 47±7 wt.-%, and in particular 35±5 wt.-%, 40±5 wt.-% or 47±5 wt.-%, based on the total weight of the monolithic pharmaceutical dosage form.

In a preferred embodiment, the content of the first pharmacologically active ingredient (A₁) is within the range of from 0.01 to 85 wt.-%, more preferably 0.1 to 55 wt.-%, still more preferably 0.5 to 32 wt.-%, based on the total weight of the first segment(s) (S₁). In another preferred embodiment, the content of the first pharmacologically active ingredient (A₁) is within the range of from 1 to 95 wt.-%, more preferably 10 to 87 wt.-%, still more preferably 17 to 82 wt.-%, based on the total weight of the first segment(s) (S₁).

In a preferred embodiment, the content of the first pharmacologically active ingredient (A₁) is within the range of from 2±1.0 wt.-%, 7±6 wt.-% or 12±11 wt.-%, more preferably 2±0.8 wt.-%, 7±5 wt.-% or 12±8 wt.-%, still more preferably 2±0.6 wt.-%, 7±4 wt.-% or 12±6 wt.-%, most preferably 2±0.4 wt.-%, 7±3 wt.-% or 12±4 wt.-%, and in particular 2±0.2 wt.-%, 7±2 wt.-% or 12±2 wt.-%, based on the total weight of the first segment(s) (S₁). In another preferred embodiment, the content of the first pharmacologically active ingredient (A₁) is within the range of from 19±15 wt.-%, 29±25 wt.-% or 40±25 wt.-%, more preferably 19±11 wt.-%, 29±18 wt.-% or 40±18 wt.-%, still more preferably 19±7 wt.-%, 29±12 wt.-% or 40±12 wt.-%, most preferably 19±4 wt.-%, 29±7 wt.-% or 40±7 wt.-%, and in particular 19±2 wt.-%, 29±5 wt.-% or 40±5 wt.-%, based on the total weight of the first segment(s) (S₁). In another preferred embodiment, the content of the first pharmacologically active ingredient (A₁) is within the range of from 50±40 wt.-%, 60±30 wt.-%, 70±20 wt.-% or 80±15 wt.-%, more preferably 50±30 wt.-%, 60±20 wt.-%, 70±15 wt.-% or 80±12 wt.-%, still more preferably 50±20 wt.-%, 60±15 wt.-%, 70±10 wt.-% or 80±9 wt.-%, most preferably 50±10 wt.-%, 60±10 wt.-%, 70±7 wt.-% or 80±7 wt.-%, and in particular 50±5 wt.-%, 60±5 wt.-%, 70±5 wt.-% or 80±5 wt.-%, based on the total weight of the first segment(s) (S₁).

The total dose of the first pharmacologically active ingredient (A₁) which is preferably contained in the first segment (S₁) and the monolithic pharmaceutical dosage form, respectively, is not limited. The dose of the first pharmacologically active ingredient (A₁) which is adapted for administration preferably is in the range of 0.01 mg to 2,000 mg or 0.01 mg to 1,000 mg or 0.1 mg to 800 or 500 mg, more preferably in the range of 1.0 mg to 600 or 400 mg, even more preferably in the range of 1.5 mg to 500 or 300 mg, and most preferably in the range of 2 mg to 400 or 250 mg.

In a preferred embodiment, the total amount of the first pharmacologically active ingredient (A₁) which is contained in the first segment (S₁) and the monolithic pharmaceutical dosage form, respectively, is within the range of from 0.01 to 200 mg, more preferably 0.1 to 150 or 190 mg, still more preferably 1.0 to 100 or 180 mg, yet more preferably 1.5 to 80 or 160 mg, most preferably 2.0 to 60 or 100 mg and in particular 2.5 to 40 or 80 mg. In another preferred embodiment, the total amount of the first pharmacologically active ingredient (A₁) which is contained in the first segment (S₁) and the monolithic pharmaceutical dosage form, respectively, is within the range of from 10 to 500 mg, more preferably 14 to 450 mg, still more preferably 17 to 400 mg, yet more preferably 20 to 350 mg, most preferably 22 to 325 mg and in particular 25 to 300 mg.

In a preferred embodiment, the first pharmacologically active ingredient (A₁) is contained in the first segment(s) (S₁) and the monolithic pharmaceutical dosage form, respectively, in a total amount of 10±5 20±5 30±5 40±5 50±5 60±5 70±5 80±5 90±5 μg, 100±5 μg, 125±25 μg, 150±25 μg, 175±25 μg, 200±25 μg, 250±50 μg, 300±50 μg, 350±50 μg, 400±50 μg, 450±50 μg, 500±50 μg, 550±50 μg, 600±50 μg, 650±50 μg, 700±50 μg, 750±50 μg, 800±50 μg, 850±50 μg, 900±50 μg, 950±50 μg, or 1000±50 μg. In another preferred embodiment, the first pharmacologically active ingredient (A₁) is contained in the first segment(s) (S₁) and the monolithic pharmaceutical dosage form, respectively, in a total amount of 3±2 mg, 7.5±5 mg, 10±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5, 140±5 mg, 150±5 mg, 160±5 mg, 170±5 mg, 180±5 mg, 190±5 mg, 200±5 mg, 210±5 mg, 220±5 mg, 230±5 mg, 240±5 mg, or 250±5 mg. In another preferred embodiment, the first pharmacologically active ingredient (A₁) is contained in the first segment (S₁) and the monolithic pharmaceutical dosage form, respectively, in a total amount of 3±1.5 mg, 5±2.5 mg, 7.5±2.5 mg, 10±2.5 mg, 15±2.5 mg, 20±2.5 mg, 25±2.5 mg, 30±2.5 mg, 35±2.5 mg, 40±2.5 mg, 45±2.5 mg, 50±2.5 mg, 55±2.5 mg, 60±2.5 mg, 65±2.5 mg, 70±2.5 mg, 75±2.5 mg, 80±2.5 mg, 85±2.5 mg, 90±2.5 mg, 95±2.5 mg, 100±2.5 mg, 105±2.5 mg, 110±2.5 mg, 115±2.5 mg, 120±2.5 mg, 125±2.5 mg, 130±2.5 mg, 135±2.5 mg, 140±2.5 mg, 145±2.5 mg, 150±2.5 mg, 155±2.5 mg, 160±2.5 mg, 165±2.5 mg, 170±2.5 mg, 175±2.5 mg, 180±2.5 mg, 185±2.5 mg, 190±2.5 mg, 195±2.5 mg, 200±2.5 mg, 205±2.5 mg, 210±2.5 mg, 215±2.5 mg, 220±2.5 mg, 225±2.5 mg, 230±2.5 mg, 235±2.5 mg, 240±2.5 mg, 245±2.5 mg, or 250±2.5 mg. In still another preferred embodiment, the first pharmacologically active ingredient (A₁) is contained in the first segment(s) (S₁) and the monolithic pharmaceutical dosage form, respectively, in a total amount of 250±10 mg, 275±10 mg, 300±10 mg, 325±10 mg, 350±10 mg, 375±10 mg, 400±10 mg, 425±10 mg, 450±10 mg, 475±10 mg, 500±10 mg, 525±10 mg, 550±10 mg, 575±10 mg or 600±10 mg.

In a particularly preferred embodiment, the first pharmacologically active ingredient (A₁) is tramadol, preferably its HCl salt, and the monolithic pharmaceutical dosage form is adapted for administration twice daily. In this embodiment, the first pharmacologically active ingredient (A₁) is preferably contained in the first segment(s) (S₁) and the monolithic pharmaceutical dosage form, respectively, in a total amount of from 2 to 300 mg. In another particularly preferred embodiment, the first pharmacologically active ingredient (A₁) is tramadol, preferably its HCl salt, and the monolithic pharmaceutical dosage form is adapted for administration once daily. In this embodiment, the first pharmacologically active ingredient (A₁) is preferably contained in the first segment(s) (S₁) and the monolithic pharmaceutical dosage form, respectively, in a total amount of from 10 to 500 mg.

In another particularly preferred embodiment, the first pharmacologically active ingredient (A₁) is oxycodone, preferably its HCl salt, and the monolithic pharmaceutical dosage form is adapted for administration twice daily. In this embodiment, the first pharmacologically active ingredient (A₁) is preferably contained in the first segment(s) (S₁) and the monolithic pharmaceutical dosage form, respectively, in a total amount of from 5 to 80 mg. In another particularly preferred embodiment, the first pharmacologically active ingredient (A₁) is oxycodone, preferably its HCl salt, and the monolithic pharmaceutical dosage form is adapted for administration once daily. In this embodiment, the first pharmacologically active ingredient (A₁) is preferably contained in the first segment(s) (S₁) and the monolithic pharmaceutical dosage form, respectively, in a total amount of from 10 to 320 mg.

In another particularly preferred embodiment, the first pharmacologically active ingredient (A₁) is oxymorphone, preferably its HCl salt, and the monolithic pharmaceutical dosage form is adapted for administration twice daily. In this embodiment, the first pharmacologically active ingredient (A₁) is preferably contained in the first segment(s) (S₁) and the monolithic pharmaceutical dosage form, respectively, in a total amount of from 5 to 40 mg. In another particularly preferred embodiment, the first pharmacologically active ingredient (A₁) is oxymorphone, preferably its HCl salt, and the monolithic pharmaceutical dosage form is adapted for administration once daily. In this embodiment, the first pharmacologically active ingredient (A₁) is preferably contained in the first segment(s) (S₁) and the monolithic pharmaceutical dosage form, respectively, in a total amount of from 10 to 80 mg.

In another particularly preferred embodiment, the first pharmacologically active ingredient (A₁) is tapentadol, preferably its HCl salt, and the monolithic pharmaceutical dosage form is adapted for administration once daily or twice daily. In this embodiment, the first pharmacologically active ingredient (A₁) is preferably contained in the first segment(s) (S₁) and the monolithic pharmaceutical dosage form, respectively, in a total amount of from 25 to 250 mg.

In still another particularly preferred embodiment, the first pharmacologically active ingredient (A₁) is hydromorphone, preferably its HCl salt, and the monolithic pharmaceutical dosage form is adapted for administration twice daily. In this embodiment, the first pharmacologically active ingredient (A₁) is preferably contained in the first segment(s) (S₁) and the monolithic pharmaceutical dosage form, respectively, in a total amount of from 2 to 52 mg. In another particularly preferred embodiment, the first pharmacologically active ingredient (A₁) is hydromorphone, preferably its HCl salt, and the monolithic pharmaceutical dosage form is adapted for administration once daily. In this embodiment, the first pharmacologically active ingredient (A₁) is preferably contained in the first segment(s) (S₁) and the monolithic pharmaceutical dosage form, respectively, in a total amount of from 4 to 104 mg.

In yet another particularly preferred embodiment, the first pharmacologically active ingredient (A₁) is hydrocodone, preferably its HCl salt, and the pharmaceutical dosage form is adapted for administration twice daily. In this embodiment, the first pharmacologically active ingredient (A₁) is preferably contained in the formed segment(s) (S₁) and the pharmaceutical dosage form, respectively, in a total amount of from 5 to 250 mg. In another particularly preferred embodiment, the first pharmacologically active ingredient (A₁) is hydrocodone, preferably its HCl salt, and the pharmaceutical dosage form is adapted for administration once daily. In this embodiment, the first pharmacologically active ingredient (A₁) is preferably contained in the formed segment(s) (S₁) and the pharmaceutical dosage form, respectively, in a total amount of from 5 to 250 mg.

In a further particularly preferred embodiment, the first pharmacologically active ingredient (A₁) is morphine, preferably its HCl or H₂SO₄ salt, and the pharmaceutical dosage form is adapted for administration twice daily. In this embodiment, the first pharmacologically active ingredient (A₁) is preferably contained in the formed segment(s) (S₁) and the pharmaceutical dosage form, respectively, in a total amount of from 5 to 250 mg. In another particularly preferred embodiment, the first pharmacologically active ingredient (A₁) is morphine, preferably its HCl or H₂SO₄ salt, and the pharmaceutical dosage form is adapted for administration once daily. In this embodiment, the first pharmacologically active ingredient (A₁) is preferably contained in the formed segment(s) (S₁) and the pharmaceutical dosage form, respectively, in a total amount of from 5 to 250 mg.

In still a further particularly preferred embodiment, the first pharmacologically active ingredient (A₁) is buprenorphine, preferably its HCl salt, and the pharmaceutical dosage form is adapted for administration twice daily. In this embodiment, the first pharmacologically active ingredient (A₁) is preferably contained in the formed segment(s) (S₁) and the pharmaceutical dosage form, respectively, in a total amount of from 1 to 12 mg. In another particularly preferred embodiment, the first pharmacologically active ingredient (A₁) is buprenorphine, preferably its HCl salt, and the pharmaceutical dosage form is adapted for administration once daily. In this embodiment, the first pharmacologically active ingredient (A₁) is preferably contained in the formed segment(s) (S₁) and the pharmaceutical dosage form, respectively, in a total amount of from 2 to 12 mg.

In another preferred embodiment, the first pharmacologically active ingredient (A₁) is paracetamol (acetaminophen). In this embodiment, the paracetamol is preferably contained in the first segment(s) (S₁) or the monolithic pharmaceutical dosage form in an amount of from 10 to 400 mg or 100 to 600 mg, more preferably 15 to 350 mg or 150 to 550 mg, still more preferably 20 to 300 mg or 200 to 500 mg, most preferably 25 to 250 mg or 250 to 450 mg and in particular 30 to 200 mg or 275 to 400 mg.

In still another preferred embodiment, the first pharmacologically active ingredient (A₁) is ibuprofen. In this embodiment, the ibuprofen is preferably contained in the first segment(s) (S₁) or the monolithic pharmaceutical dosage form in an amount of from 100 to 600 mg, more preferably 150 to 550 mg, still more preferably 200 to 500 mg, most preferably 250 to 450 mg and in particular 275 to 400 mg.

The first pharmacologically active ingredient (A₁) that is preferably employed in the preparation of the first segment(s) (S₁) preferably has an average particle size of less than 500 microns, still more preferably less than 300 microns, yet more preferably less than 200 or 100 microns. There is no lower limit on the average particle size and it may be, for example, 50 microns. The particle size of pharmacologically active ingredients may be determined by any technique conventional in the art, e.g. laser light scattering, sieve analysis, light microscopy or image analysis.

In a preferred embodiment, the segment (S₁) provides immediate release of the first pharmacologically active ingredient (A₁).

When the segment (S₁) provides immediate release of the first pharmacologically active ingredient (A₁), the first segment(s) (S₁) preferably comprise(s) an immediate release matrix. The immediate release matrix in turn preferably comprises an immediate release matrix material that serves the function of providing immediate release of the first pharmacologically active ingredient (A₁), optionally further pharmaceutical excipients that do not substantially influence the release profile, and the first pharmacologically active ingredient (A₁).

The first pharmacologically active ingredient (A₁) is preferably embedded, particularly preferably dispersed in the immediate release matrix material.

The total content of the immediate release matrix (first pharmacologically active ingredient (A₁)+immediate release matrix material+optionally present excipients that do not substantially influence the release profile) that is contained in the first segment(s) (S₁) is preferably at least 30 wt.-%, more preferably at least 40 wt.-%, still more preferably at least 50 wt.-%, yet more preferably at least 60 wt.-%, even more preferably at least 70 wt.-%, most preferably at least 80 wt.-%, and in particular at least 90 wt.-%, relative to the total weight of the first segment(s) (S₁).

The total content of the immediate release matrix (first pharmacologically active ingredient (A₁)+immediate release matrix material+optionally present excipients that do not substantially influence the release profile) that is contained in the first segment(s) (S₁) is preferably the range of from 5 to 95 wt.-%, more preferably 15 to 90 wt.-%, still more preferably 25 to 88 wt.-%, yet more preferably 35 to 86 wt.-%, even more preferably 40 to 84 wt.-%, most preferably 45 to 82 wt.-%, and in particular 50 to 80 wt.-%, relative to the total weight of the monolithic pharmaceutical dosage form.

Preferably, the first pharmacologically active ingredient (A₁) and the immediate release matrix material are intimately homogeneously distributed within the first segment(s) (S₁) so that the first segment(s) (S₁) do(es) not contain any portions where either the first pharmacologically active ingredient (A₁) is present in the absence of immediate release matrix material or where immediate release matrix material is present in the absence of the first pharmacologically active ingredient (A₁).

When the first segment (S₁) is film coated, the immediate release matrix material is preferably homogeneously distributed in the body of the first segment (S₁), i.e. the film coating preferably does not contain immediate release matrix material.

The chemical nature and the content of the immediate release matrix material are not particularly limited. The skilled person will readily be able to determine appropriate immediate release matrix materials as well as their appropriate quantities.

In a preferred embodiment, suitable immediate release matrix materials also include fillers/binders. Suitable fillers/binders are those disclosed herein below in connection with the excipients which may be contained in the segment (S₁) which provides immediate release of the first pharmacologically active ingredient (A₁).

Particularly preferred immediate release matrix materials include but are not limited to polyvinyl alcohol-polyethylene glycol graft copolymers and acrylic polymers (preferably copolymers of one or two different C₁₋₄-alkyl(meth)acrylate monomers and dimethyl-ammonioethyl(meth)acrylate).

Preferred immediate release matrix materials which are commercially available include Kollicoat®IR, Eudragit® E PO and Eudragit® E 100.

When the first segment(s) (S₁) comprises an immediate release matrix material, the first segment(s) (S₁) preferably further contain(s) conventional pharmaceutical excipients that do not substantially influence the release profile.

Preferably, the total content of the immediate release matrix material, i.e. material that serves the function of providing immediate release of the first pharmacologically active ingredient (A₁), is within the range of from 5 to 95 wt.-%, more preferably from 7 to 80 wt.-%, still more preferably from 9 to 75 wt.-%, yet more preferably from 11 to 70 wt.-%, most preferably from 13 to 65 wt.-% and in particular from 15 to 60 wt.-%, relative to the total weight of the first segment(s) (S₁). When the monolithic pharmaceutical dosage form contains more than one first segment (S₁), e.g. when the dosage form is a layered tablet and contains two layers of the first segment (S₁), these percent values preferably are related to the total weight of all first segments (S₁) which are contained in the monolithic pharmaceutical dosage form, e.g. the combined weight of the two layers of the first segment (S₁).

Preferably, the total content of the immediate release matrix material, i.e. material that serves the function of providing immediate release of the first pharmacologically active ingredient (A₁), contained in the first segment(s) (S₁) is within the range of from 1 to 95 wt.-%, more preferably from 5 to 80 wt.-%, still more preferably from 7 to 65 wt.-%, yet more preferably from 8 to 50 wt.-%, most preferably from 9 to 40 wt.-% and in particular from 10 to 30 wt.-%, relative to the total weight of the monolithic pharmaceutical dosage form.

Preferably, the relative weight ratio of the immediate release matrix material, i.e. material that serves the function of providing immediate release of the first pharmacologically active ingredient (A₁), to the first pharmacologically active ingredient (A₁) is within the range of from 20:1 to 1:20, more preferably 15:1 to 1:15, still more preferably 10:1 to 1:10, yet more preferably 5:1 to 1:8, most preferably 3:1 to 1:6 and in particular 1:1 to 1:5.

When the first segment(s) (S₁) comprises an immediate release matrix, it may optionally comprise conventional pharmaceutical excipients.

Preferably, when comprising an immediate release matrix, the first segment(s) (S₁) further contain(s) a filler or a binder. As many fillers can be regarded as binders and vice versa, for the purpose of the specification “filler/binder” refers to any excipient that is suitable as filler, binder or both. Thus, the first segment(s) (S₁) preferably providing immediate release of the first pharmacologically active ingredient (A₁) preferably comprise(s) a filler/binder. In a preferred embodiment, the filler/binders can be regarded as immediate release matrix materials.

Preferred fillers (=filler/binders) are selected from the group consisting of poloxamers (e.g. Lutrol® F68), silicium dioxide (e.g. Aerosil®), microcrystalline cellulose (e.g. Avicel®, Elcema®, Emocel®, ExCel®, Vitacell®); cellulose ether (e.g. Natrosol®, Klucel®, Methocel®, Blanose®, Pharmacoat®, Viscontran^{k}); mannitol; dextrines; dextrose; calciumhydrogen phosphate (e.g. Emcompress®); tricalcium phosphate, maltodextrine (e.g. Emdex®); lactose (e.g. Fast-Flow Lactose®; Ludipress®° Pharmaceutical dosage Formtose®, Zeparox®); polyvinylpyrrolidone (PVP) (e.g. Kollidone®, Polyplasdone®, Polydone®); saccharose (e.g. Nu-Tab®, Sugar Tab®); magnesium salts (e.g. MgCO₃, MgO, MgSiO₃); starches and pretreated starches (e.g. Prejel®, Primotab® ET, Starch® 1500).

Some fillers/binders may also serve other purposes. It is known, for example, that silicium dioxide exhibits excellent function as a glidant. Preferably, the first segment(s) (S₁) comprise(s) a glidant such as silicium dioxide.

In a preferred embodiment, the content of the filler/binder or mixture of fillers/binders in the first segment(s) (S₁) is from 0 to 90 wt.-%, more preferably 1 to 80 wt.-%, still more preferably 2 to 70 wt.-%, yet more preferably 3 to 60 wt.-%, most preferably 4 to 55 wt.-%, and in particular from 5 to 50 wt.-%, based on the total weight of the first segment(s) (S₁).

Preferably, when comprising an immediate release matrix, the first segment(s) (S₁) further contain(s) a diluent or lubricant, preferably selected from the group consisting of calcium stearate; magnesium stearate; glycerol monobehenate (e.g. Compritol®); Myvatex®; Precirol®; Precirol® AtoS; sodium stearylfumarate (e.g. Pruv®); and talcum. Preferably, the content of the lubricant in the first segment(s) (S₁) is at most 10.0 wt.-%, more preferably at most 7.5 wt.-%, still more preferably at most 5.0 wt.-%, yet more preferably at most 2.0 wt.-%, even more preferably at most 1.0 wt.-%, and most preferably at most 0.5 wt.-%, based on the total weight of the first segment(s) (S₁) or based on the total weight of pharmaceutical dosage form.

The first segment(s) (S₁) of the monolithic pharmaceutical dosage form according to the invention may additionally contain other excipients that are conventional in the art, e.g. diluents, binders, granulating aids, colorants, flavourants, glidants, wet-regulating agents and disintegrants. The skilled person will readily be able to determine appropriate quantities of each of these excipients.

In a particularly preferred embodiment, when the first segment(s) (S₁) provides immediate release of the pharmacologically active ingredient (S₁), said first segment(s) (S₁) do(es) not contain one or more gel-forming agents and/or a silicone. According to this embodiment, the first segment(s) (S₁) of the monolithic pharmaceutical dosage form according to the invention preferably do(es) not contain polyalkylene oxides, acrylic polymers or waxy materials. If the first segment(s) (S₁) provides immediate release of the first pharmacologically active ingredient (A₁) and contain(s) polyalkylene oxides, acrylic polymers and/or waxy materials, the total content of polyalkylene oxides, acrylic polymers and waxy materials preferably is not more than 30 wt.-%, more preferably not more than 25 wt.-%, still more preferably not more than 20 wt.-%, yet more preferably not more than 15 wt.-%, even more preferably not more than 10 wt.-%, most preferably not more than 5.0 wt.-%, and in particular not more than 1.0 wt.-%, relative to the total weight of the first segment(s) (S₁).

As used herein the term “gel-forming agent” is used to refer to a compound that, upon contact with a solvent (e.g. water), absorbs the solvent and swells, thereby forming a viscous or semi-viscous substance. Preferred gel-forming agents are not cross-linked. This substance may moderate pharmacologically active ingredient release from the segments in both aqueous and aqueous alcoholic media. Upon full hydration, a thick viscous solution or dispersion is typically produced that significantly reduces and/or minimizes the amount of free solvent which can contain an amount of solubilized pharmacologically active ingredient, and which can be drawn into a syringe. The gel that is formed may also reduce the overall amount of pharmacologically active ingredient extractable with the solvent by entrapping the pharmacologically active ingredient within a gel structure. Thus the gel-forming agent may play an important role in conferring tamper-resistance to the pharmaceutical dosage forms according to the invention.

When the first segment(s) (S₁) provides immediate release of the first pharmacologically active ingredient (A₁), gel-forming agents that preferably are not contained in said first segment(s) (S₁) include pharmaceutically acceptable polymers, typically hydrophilic polymers, such as hydrogels. Representative examples of gel-forming agent include polyalkylene oxide such as polyethylene oxide, polyvinyl alcohol, hydroxypropylmethyl cellulose, carbomers, poly(uronic) acids and mixtures thereof.

Preferred contents of the first pharmacologically active ingredient (A₁), immediate release matrix material, and excipients, relative to the total weight of the first segment(s) (S₁), are summarized as embodiments B¹ to B¹⁶ in the tables here below:

| | wt.-% | | | |
|---|---|---|---|---|
| | B¹ | B² | B³ | B⁴ |
| first pharmacologically active | 80 ± 70 | 80 ± 50 | 80 ± 30 | 80 ± 10 |
| ingredient (A₁) | | | | |
| immediate release matrix material | 10 ± 10 | 10 ± 8 | 10 ± 6 | 10 ± 5 |
| pharmaceutical excipients | 20 ± 20 | 20 ± 20 | 20 ± 20 | 20 ± 20 |

| | wt.-% | | | |
|---|---|---|---|---|
| | B⁵ | B⁶ | B⁷ | B⁸ |
| first pharmacologically active | 70 ± 60 | 70 ± 50 | 70 ± 30 | 70 ± 10 |
| ingredient (A₁) | | | | |
| immediate release matrix material | 20 ± 20 | 20 ± 15 | 20 ± 10 | 20 ± 5 |
| pharmaceutical excipients | 20 ± 20 | 20 ± 20 | 20 ± 20 | 20 ± 20 |

| | wt.-% | | | |
|---|---|---|---|---|
| | B⁹ | B¹⁰ | B¹¹ | B¹² |
| first pharmacologically active | 60 ± 50 | 60 ± 30 | 60 ± 20 | 60 ± 10 |
| ingredient (A₁) | | | | |
| immediate release matrix material | 30 ± 30 | 30 ± 20 | 30 ± 10 | 30 ± 5 |
| pharmaceutical excipients | 20 ± 20 | 20 ± 20 | 20 ± 20 | 20 ± 20 |

| | wt.-% | | | |
|---|---|---|---|---|
| | B¹³ | B¹⁴ | B¹⁵ | B¹⁶ |
| first pharmacologically active | 50 ± 40 | 50 ± 30 | 50 ± 20 | 50 ± 10 |
| ingredient (A₁) | | | | |
| immediate release matrix material | 40 ± 40 | 40 ± 30 | 40 ± 20 | 40 ± 10 |
| pharmaceutical excipients | 20 ± 20 | 20 ± 20 | 20 ± 20 | 20 ± 20 |

In a preferred embodiment, the first segment(s) (S₁) provide(s) immediate release of the first pharmacologically active ingredient (A₁). Preferably, the immediate release matrix provides for an immediate release of the first pharmacologically active ingredient (A₁) from the first segment (S₁).

Preferably, under in vitro conditions the monolithic pharmaceutical dosage form has released after 15 minutes 20 to 90%, after 30 minutes 40 to 99%, after 45 minutes 80 to 99% and after 60 minutes more than 95% of the first pharmacologically active ingredient (A₁).

Suitable in vitro conditions are known to the skilled artisan. In this regard it can be referred to, e.g., the Eur. Ph. Preferably, the release profile is measured under the following conditions: Paddle apparatus, 50 rpm, 37±5° C., 900 mL 0.1 M HCl (pH 1.0) or simulated intestinal fluid pH 6.8 (phosphate buffer) or pH 4.5. In another preferred embodiment, the rotational speed of the paddle is increased to 75 rpm. In another preferred embodiment, the release profile is determined under the following conditions: basket method, 75 rpm, 37±5° C., 900 mL 0.1 N HCl or 900 mL of SIF sp (pH 6.8) or 900 mL of 0.1 N HCl+40% ethanol.

Preferred release profiles R¹ to R⁵ are summarized in the table here below [all data in wt.-% of released first pharmacologically active ingredient (A₁)]:

| time | R¹ | R² | R³ | R⁴ | R⁵ |
|---|---|---|---|---|---|
| 15 min | 10-30 | 10-40 | 20-50 | 20-60 | 20-70 |
| 30 min | 40-70 | 60-99 | 70-90 | 60-90 | 60-99 |
| 45 min | 70-90 | 85-99 | 85-99 | 70-99 | 80-99.9 |
| 60 min | 80-99 | 90-99.9 | 90-99.9 | 90-99.9 | >99 |
| 120 min | >99 | >95 | >95 | 90-99.9 | |

Further preferred release profiles R⁶ to R⁹ are summarized in the table here below [all data in wt.-% of released first pharmacologically active ingredient (A₁)]:

| time | R⁶ | R⁷ | R⁸ | R⁹ |
|---|---|---|---|---|
| 15 min | 43 ± 10 | 50 ± 10 | 55 ± 10 | 65 ± 10 |
| 30 min | 89 ± 10 | 83 ± 10 | 80 ± 10 | 93 ± 7 |
| 45 min | 94 ± 6 | 95 ± 5 | 88 ± 12 | 97 ± 3 |
| 60 min | 95 ± 5 | 97 ± 3 | 90 ± 10 | 99 ± 1 |
| 120 min | 98 ± 2 | 98 ± 2 | 90 ± 10 | |

In a particularly preferred embodiment; under in vitro conditions in 900 mL 0.1 N HCl (pH 1.0), using the paddle method according to Ph. Eur. at 50 rpm, after 30 min under physiological conditions, the monolithic pharmaceutical dosage form has released at least 30% or at least 40%, more preferably at least 50%, still more preferably at least 60%, yet more preferably at least 70%, most preferably at least 75% and in particular at least 80% of the first pharmacologically active ingredient (A₁) relative to the total amount of the first pharmacologically active ingredient (A₁) originally contained in the pharmaceutical dosage form.

In another preferred embodiment, the segment (S₁) provides prolonged release of the first pharmacologically active ingredient (A₁).

While such prolonged release may principally be achieved by providing the first segment(s) (S₁) with a prolonged release coating containing pore formers, prolonged release is preferably achieved by a prolonged release matrix.

Thus, the first segment(s) (S₁) preferably comprise(s) a prolonged release matrix. The prolonged release matrix in turn preferably comprises a prolonged release matrix material that serves the function of providing prolonged release of the first pharmacologically active ingredient (A₁), optionally further pharmaceutical excipients that do not substantially influence the release profile, and the first pharmacologically active ingredient (A₁).

The first pharmacologically active ingredient (A₁) is preferably embedded, particularly preferably dispersed in the prolonged release matrix material.

Preferably, the total content of the prolonged release matrix (first pharmacologically active ingredient (A₁)+prolonged release matrix material+optionally present excipients that do not substantially influence the release profile) that is contained in the first segment(s) (S₁) is preferably at least 30 wt.-%, more preferably at least 40 wt.-%, still more preferably at least 50 wt.-%, yet more preferably at least 60 wt.-%, even more preferably at least 70 wt.-%, most preferably at least 80 wt.-%, and in particular at least 90 wt.-%, relative to the total weight of the first segment(s) (S₁).

Preferably, the total content of the prolonged release matrix (first pharmacologically active ingredient (A₁)+prolonged release matrix material+optionally present excipients that do not substantially influence the release profile) that is contained in the first segment(s) (S₁) is preferably within the range of from 5 to 95 wt.-%, more preferably 8 to 90 wt.-%, still more preferably 11 to 80 wt.-%, yet more preferably 14 to 70 wt.-%, even more preferably 16 to 60 wt.-%, most preferably 18 to 50 wt.-%, and in particular 20 to 45 wt.-%, relative to the total weight of the monolithic pharmaceutical dosage form.

Preferably, the first pharmacologically active ingredient (A₁) and the prolonged release matrix material are intimately homogeneously distributed within the first segment(s) (S₁) so that the first segment(s) (S₁) do(es) not contain any portions where either the first pharmacologically active ingredient (A₁) is present in the absence of prolonged release matrix material or where prolonged release matrix material is present in the absence of the first pharmacologically active ingredient (A₁).

When the first segment (S₁) is film coated, the prolonged release matrix material is preferably homogeneously distributed in the body of the first segment (S₁), i.e. the film coating preferably does not contain prolonged release matrix material.

When the first segment(s) (S₁) comprises a prolonged release matrix material, the first segment(s) (S₁) preferably contain(s) conventional pharmaceutical excipients that do not substantially influence the release profile.

Preferably, the total content of the prolonged release matrix material, i.e. material that preferably serves the function of providing prolonged release of the first pharmacologically active ingredient (A₁), is within the range of from 20 to 95 wt.-%, relative to the total weight of the first segment(s) (S₁). When the monolithic pharmaceutical dosage form contains more than one first segment (S₁), e.g. when the dosage form is a layered tablet and contains two layers of the first segment (S₁), these percent values preferably are related to the total weight of all first segments (S₁) which are contained in the monolithic pharmaceutical dosage form, e.g. the combined weight of the two layers of the first segment (S₁).

In a preferred embodiment, the content of the prolonged release matrix material is at least 5 wt.-%, or at least 10 wt.-%, or at least 15 wt.-%, more preferably at least 20 wt.-%, or at least 25 wt.-%, or at least 30 wt.-%, still more preferably at least 35 wt.-%, or at least 40 wt.-%, or at least 45 wt.-%, yet more preferably at least 50 wt.-%, or at least 55 wt.-%, or at least 60 wt.-%, most preferably at least 65 wt.-%, or at least 70 wt.-%, or at least 75 wt.-%, and in particular at least 80 wt.-%, or at least 85 wt.-%, or at least 90 wt.-%, based on the total weight of the first segment(s) (S₁).

In a preferred embodiment, the total content of prolonged release matrix material is within the range of 25±20 wt.-%, more preferably 25±15 wt.-%, most preferably 25±10 wt.-%, and in particular 25±5 wt.-%, based on the total weight of the first segment(s) (S₁).

In another preferred embodiment, the total content of prolonged release matrix material is within the range of 30±20 wt.-%, more preferably 30±15 wt.-%, most preferably 30±10 wt.-%, and in particular 30±5 wt.-%, based on the total weight of the first segment(s) (S₁).

In still another preferred embodiment, the total content of prolonged release matrix material is within the range of 35±20 wt.-%, more preferably 35±15 wt.-%, most preferably 35±10 wt.-%, and in particular 35±5 wt.-%, based on the total weight of the first segment(s) (S₁).

In a yet another preferred embodiment, the total content of prolonged release matrix material is within the range of 40±20 wt.-%, more preferably 40±15 wt.-%, and most preferably 40±10 wt.-%, and in particular 40±5 wt.-%, based on the total weight of the first segment(s) (S₁).

In a further preferred embodiment, the total content of prolonged release matrix material is within the range of 45±20 wt.-%, more preferably 45±15 wt.-%, and most preferably 45±10 wt.-%, and in particular 45±5 wt.-%, based on the total weight of the first segment(s) (S₁).

In still a further preferred embodiment, the total content of prolonged release matrix material is within the range of 50±20 wt.-%, more preferably 50±15 wt.-%, and most preferably 50±10 wt.-%, and in particular 50±5 wt.-%, based on the total weight of the first segment(s) (S₁).

In yet a further preferred embodiment, the total content of prolonged release matrix material is within the range of 55±20 wt.-%, more preferably 55±15 wt.-%, and most preferably 55±10 wt.-%, and in particular 55±5 wt.-%, based on the total weight of the first segment(s) (S₁).

In another preferred embodiment, the total content of prolonged release matrix material is within the range of 60±20 wt.-%, more preferably 60±15 wt.-%, and most preferably 60±10 wt.-%, and in particular 60±5 wt.-%, based on the total weight of the first segment(s) (S₁).

In still another preferred embodiment, the total content of prolonged release matrix is within the range of 65±20 wt.-%, more preferably 65±15 wt.-%, and most preferably 65±10 wt.-%, and in particular 65±5 wt.-%, based on the total weight of the first segment(s) (S₁).

In yet another preferred embodiment, the total content of prolonged release matrix material is within the range of 70±20 wt.-%, more preferably 70±15 wt.-%, and most preferably 70±10 wt.-%, and in particular 70±5 wt.-%, based on the total weight of the first segment(s) (S₁).

In a further preferred embodiment, the total content of prolonged release matrix material is within the range of 75±20 wt.-%, more preferably 75±15 wt.-%, and most preferably 75±10 wt.-%, and in particular 75±5 wt.-%, based on the total weight of the first segment(s) (S₁).

In still a further preferred embodiment, the total content of prolonged release matrix material is within the range of 80±15 wt.-%, more preferably 80±12 wt.-%, and most preferably 80±10 wt.-%, and in particular 80±5 wt.-%, based on the total weight of the first segment(s) (S₁).

In yet a further preferred embodiment, the total content of prolonged release matrix material is within the range of 85±10 wt.-%, more preferably 85±8 wt.-%, and most preferably 85±6 wt.-%, and in particular 85±4 wt.-%, based on the total weight of the first segment(s) (S₁).

In another preferred embodiment, the total content of prolonged release matrix material is within the range of 90±8 wt.-%, more preferably 90±7 wt.-%, and most preferably 90±6 wt.-%, and in particular 90±4 wt.-%, based on the total weight of the first segment(s) (S₁).

In still another preferred embodiment, the total content of prolonged release matrix material is within the range of 95±3 wt.-%, more preferably 95±2 wt.-%, and most preferably 95±1 wt.-%, and in particular 95±0.5 wt.-%, based on the total weight of the first segment(s) (S₁).

Preferably, the total content of the prolonged release matrix material, i.e. material that preferably serves the function of providing prolonged release of the first pharmacologically active ingredient (A₁), which may be contained in the first segment(s) (S₁) is within the range of from 5 to 95 wt.-%, more preferably 20 to 80 wt.-% relative to the total weight of the monolithic pharmaceutical dosage form.

In a preferred embodiment, the total content of the prolonged release matrix material is at least 5 wt.-% or at least 10 wt.-%, more preferably at least 15 wt.-%, still more preferably at least 20 wt.-%, yet more preferably at least 25 wt.-% and in particular at least 30 wt.-%, or at least 35 wt.-%, or at least 40 wt.-%, or at least 45 wt.-%, or at least 50 wt.-%, or at least 55 wt.-%, or at least 60 wt.-%, based on the total weight of the monolithic pharmaceutical dosage form.

In a preferred embodiment, the total content of prolonged release matrix material is within the range of 10±5 wt.-%, more preferably 10±4 wt.-%, most preferably 10±3 wt.-%, and in particular 10±2 wt.-%, based on the total weight of the monolithic pharmaceutical dosage form.

In another preferred embodiment, the total content of prolonged release matrix material is within the range of 15±10 wt.-%, more preferably 15±7 wt.-%, most preferably 15±5 wt.-%, and in particular 15±3 wt.-%, based on the total weight of the monolithic pharmaceutical dosage form.

In still another preferred embodiment, the total content of prolonged release matrix material is within the range of 20±16 wt.-%, more preferably 20±12 wt.-%, most preferably 20±8 wt.-%, and in particular 20±4 wt.-%, based on the total weight of the monolithic pharmaceutical dosage form.

In yet another preferred embodiment, the total content of prolonged release matrix material is within the range of 25±20 wt.-%, more preferably 25±15 wt.-%, most preferably 25±10 wt.-%, and in particular 25±5 wt.-%, based on the total weight of the monolithic pharmaceutical dosage form.

In a further preferred embodiment, the total content of prolonged release matrix material is within the range of 30±20 wt.-%, more preferably 30±15 wt.-%, most preferably 30±10 wt.-%, and in particular 30±5 wt.-%, based on the total weight of the monolithic pharmaceutical dosage form.

In still a further preferred embodiment, the total content of prolonged release matrix material is within the range of 35±20 wt.-%, more preferably 35±15 wt.-%, most preferably 35±10 wt.-%, and in particular 35±5 wt.-%, based on the total weight of the monolithic pharmaceutical dosage form.

In a still further preferred embodiment, the total content of prolonged release matrix material is within the range of 40±20 wt.-%, more preferably 40±15 wt.-%, and most preferably 40±10 wt.-%, and in particular 40±5 wt.-%, based on the total weight of the monolithic pharmaceutical dosage form.

In a yet further preferred embodiment, the total content of prolonged release matrix material is within the range of 45±20 wt.-%, more preferably 45±15 wt.-%, and most preferably 45±10 wt.-%, and in particular 45±5 wt.-%, based on the total weight of the monolithic pharmaceutical dosage form.

In another preferred embodiment, the total content of prolonged release matrix material is within the range of 50±20 wt.-%, more preferably 50±15 wt.-%, and most preferably 50±10 wt.-%, and in particular 50±5 wt.-%, based on the total weight of the monolithic pharmaceutical dosage form.

In a yet further preferred embodiment, the total content of prolonged release matrix material is within the range of 55±20 wt.-%, more preferably 55±15 wt.-%, and most preferably 55±10 wt.-%, and in particular 55±5 wt.-%, based on the total weight of the monolithic pharmaceutical dosage form.

In another preferred embodiment, the total content of prolonged release matrix material is within the range of 60±20 wt.-%, more preferably 60±15 wt.-%, and most preferably 60±10 wt.-%, and in particular 60±5 wt.-%, based on the total weight of the monolithic pharmaceutical dosage form.

In still another preferred embodiment, the total content of prolonged release matrix material is within the range of 65±20 wt.-%, more preferably 65±15 wt.-%, and most preferably 65±10 wt.-%, and in particular 65±5 wt.-%, based on the total weight of the monolithic pharmaceutical dosage form.

Preferably, the relative weight ratio of the prolonged release matrix material, i.e. material that preferably serves the function of providing prolonged release of the first pharmacologically active ingredient (A₁), to the first pharmacologically active ingredient (A₁) is within the range of from 50:1 to 1:20 or 20:1 to 1:20, more preferably 45:1 to 1:15 or 15:1 to 1:15, still more preferably 40:1 to 1:10 or 10:1 to 1:10, yet more preferably 37:1 to 1:7 or 7:1 to 1:7, most preferably 33:1 to 1:5 or 5:1 to 1:5, and in particular 32:1 to 1:2 or 2:1 to 1:2.

The prolonged release matrix material, i.e. material that preferably serves the function of providing prolonged release of the first pharmacologically active ingredient (A₁), preferably comprises at least one synthetic or natural polymer (C) and/or optionally a waxy material. Preferably, the prolonged release matrix material comprises only one synthetic or natural polymer (C). In a preferred embodiment, the prolonged release matrix material consists of synthetic or natural polymer (C).

In a preferred embodiment, the segment (S₁) and/or the segment (S₂) contains a pharmacologically active ingredient (A₁) and (A₂), respectively, which is embedded in a matrix material comprising a synthetic or natural polymer (C).

In a preferred embodiment, the segment (S₁) contains a pharmacologically active ingredient (A₁) which is embedded in a matrix material comprising a synthetic or natural polymer (C).

In another preferred embodiment, the first pharmacologically active ingredient (A₁) is embedded in a prolonged release matrix comprising a synthetic or natural polymer (C).

The total content of the synthetic or natural polymer (C) is preferably at least 65 wt.-%, more preferably at least 70 wt.-%, still more preferably at least 75 wt.-%, yet more preferably at least 80 wt.-%, even more preferably at least 85 wt.-%, most preferably at least 90 wt.-%, and in particular at least 95 wt.-%, relative to the total weight of the prolonged release matrix material, i.e. material that preferably serves the function of providing prolonged release of the first pharmacologically active ingredient (A₁).

The total content of the synthetic or natural polymer (C) is preferably at least 10 wt.-% or at least 20 wt.-%, more preferably at least 30 wt.-%, still more preferably at least 40 wt.-%, yet more preferably at least 50 wt.-%, even more preferably at least 60 wt.-%, most preferably at least 70 wt.-%, and in particular at least 80 wt.-%, relative to the total weight of the prolonged release matrix (first pharmacologically active ingredient (A₁)+prolonged release matrix material+optionally present excipients that do not substantially influence the release profile).

Preferably, the total content of the synthetic or natural polymer (C) is at least 10 wt.-% or at least 20 wt.-%, more preferably at least 30 wt.-%, still more preferably at least 40 wt.-%, yet more preferably at least 50 wt.-%, most preferably at least 60 wt.-%, and in particular at least 75 wt.-%, relative to the total weight of the first segment(s) (S₁).

In a preferred embodiment, the total content of the synthetic or natural polymer (C) is at least 5 wt.-%, more preferably at least 10 wt.-%, still more preferably at least 15 wt.-%, yet more preferably at least 20 wt.-% and in particular at least 25 wt.-%, relative to the total weight of the first segment(s) (S₁). In a particularly preferred embodiment, the content of the synthetic or natural polymer (C) is at least 30 wt.-% relative to the total weight of the first segment(s) (S₁).

In a preferred embodiment, the total content of the synthetic or natural polymer (C) is within the range of 10±8 wt.-%, more preferably 10±6 wt.-%, most preferably 10±4 wt.-%, and in particular 10±2 wt.-%, based on the total weight of the first segment(s) (S₁).

In another preferred embodiment, the total content of the synthetic or natural polymer (C) is within the range of 15±12 wt.-%, more preferably 15±10 wt.-%, most preferably 15±7 wt.-%, and in particular 15±3 wt.-%, based on the total weight of the first segment(s) (S₁).

In still another preferred embodiment, the total content of the synthetic or natural polymer (C) is within the range of 20±16 wt.-%, more preferably 20±12 wt.-%, most preferably 20±8 wt.-%, and in particular 20±4 wt.-%, based on the total weight of the first segment(s) (S₁).

In yet another preferred embodiment, the total content of the synthetic or natural polymer (C) is within the range of 25±20 wt.-%, more preferably 25±15 wt.-%, most preferably 25±10 wt.-%, and in particular 25±5 wt.-%, based on the total weight of the first segment(s) (S₁).

In a further preferred embodiment, the total content of the synthetic or natural polymer (C) is within the range of 30±20 wt.-%, more preferably 30±15 wt.-%, most preferably 30±10 wt.-%, and in particular 30±5 wt.-%, based on the total weight of the first segment(s) (S₁).

In still a further preferred embodiment, the total content of the synthetic or natural polymer (C) is within the range of 35±20 wt.-%, more preferably 35±15 wt.-%, most preferably 35±10 wt.-%, and in particular 35±5 wt.-%, based on the total weight of the first segment(s) (S₁).

In a still further preferred embodiment, the total content of the synthetic or natural polymer (C) is within the range of 40±20 wt.-%, more preferably 40±15 wt.-%, and most preferably 40±10 wt.-%, and in particular 40±5 wt.-%, based on the total weight of the first segment(s) (S₁).

In a yet further preferred embodiment, the total content of the synthetic or natural polymer (C) is within the range of 45±20 wt.-%, more preferably 45±15 wt.-%, and most preferably 45±10 wt.-%, and in particular 45±5 wt.-%, based on the total weight of the first segment(s) (S₁).

In another preferred embodiment, the total content of the synthetic or natural polymer (C) is within the range of 50±20 wt.-%, more preferably 50±15 wt.-%, and most preferably 50±10 wt.-%, and in particular 50±5 wt.-%, based on the total weight of the first segment(s) (S₁).

In a yet further preferred embodiment, the total content of the synthetic or natural polymer (C) is within the range of 55±20 wt.-%, more preferably 55±15 wt.-%, and most preferably 55±10 wt.-%, and in particular 55±5 wt.-%, based on the total weight of the first segment(s) (S₁).

In another preferred embodiment, the total content of the synthetic or natural polymer (C) is within the range of 60±20 wt.-%, more preferably 60±15 wt.-%, and most preferably 60±10 wt.-%, and in particular 60±5 wt.-%, based on the total weight of the first segment(s) (S₁).

In a yet further preferred embodiment, the total content of the synthetic or natural polymer (C) is within the range of 65±20 wt.-%, more preferably 65±15 wt.-%, and most preferably 65±10 wt.-%, and in particular 65±5 wt.-%, based on the total weight of the first segment(s) (S₁).

In another preferred embodiment, the total content of the synthetic or natural polymer (C) is within the range of 70±20 wt.-%, more preferably 70±15 wt.-%, and most preferably 70±10 wt.-%, and in particular 70±5 wt.-%, based on the total weight of the first segment(s) (S₁).

Preferably, the total content of the polymer (C) is within the range of from 1 to 99 wt.-%, more preferably 3 to 90 wt.-%, still more preferably 5 to 80 wt.-%, yet more preferably 7 to 75 wt.-%, most preferably 8 to 70 wt.-% and in particular 9 to 65 wt.-%, based on the total weight of the monolithic pharmaceutical dosage form.

In a preferred embodiment, the total content of the polymer (C) is at least 2 wt.-%, more preferably at least 5 wt.-%, most preferably at least 10 wt.-%, and in particular at least 11 wt.-%, based on the total weight of the monolithic pharmaceutical dosage form.

In a preferred embodiment, the total content of the synthetic or natural polymer (C) is within the range of 10±8 wt.-%, more preferably 10±6 wt.-%, most preferably 10±4 wt.-%, and in particular 10±2 wt.-%, based on the total weight of the monolithic pharmaceutical dosage form.

In another preferred embodiment, the total content of the synthetic or natural polymer (C) is within the range of 15±12 wt.-%, more preferably 15±10 wt.-%, most preferably 15±7 wt.-%, and in particular 15±3 wt.-%, based on the total weight of the monolithic pharmaceutical dosage form.

In still another preferred embodiment, the total content of the synthetic or natural polymer (C) is within the range of 20±16 wt.-%, more preferably 20±12 wt.-%, most preferably 20±8 wt.-%, and in particular 20±4 wt.-%, based on the total weight of the monolithic pharmaceutical dosage form.

In yet another preferred embodiment, the total content of the synthetic or natural polymer (C) is within the range of 25±20 wt.-%, more preferably 25±15 wt.-%, most preferably 25±10 wt.-%, and in particular 25±5 wt.-%, based on the total weight of the monolithic pharmaceutical dosage form.

In a further preferred embodiment, the total content of the synthetic or natural polymer (C) is within the range of 30±20 wt.-%, more preferably 30±15 wt.-%, most preferably 30±10 wt.-%, and in particular 30±5 wt.-%, based on the total weight of the monolithic pharmaceutical dosage form.

In still a further preferred embodiment, the total content of the synthetic or natural polymer (C) is within the range of 35±20 wt.-%, more preferably 35±15 wt.-%, most preferably 35±10 wt.-%, and in particular 35±5 wt.-%, based on the total weight of the monolithic pharmaceutical dosage form.

In a still further preferred embodiment, the total content of the synthetic or natural polymer (C) is within the range of 40±20 wt.-%, more preferably 40±15 wt.-%, and most preferably 40±10 wt.-%, and in particular 40±5 wt.-%, based on the total weight of the monolithic pharmaceutical dosage form.

In a yet further preferred embodiment, the total content of the synthetic or natural polymer (C) is within the range of 45±20 wt.-%, more preferably 45±15 wt.-%, and most preferably 45±10 wt.-%, and in particular 45±5 wt.-%, based on the total weight of the monolithic pharmaceutical dosage form.

In another preferred embodiment, the total content of the synthetic or natural polymer (C) is within the range of 50±20 wt.-%, more preferably 50±15 wt.-%, and most preferably 50±10 wt.-%, and in particular 50±5 wt.-%, based on the total weight of the monolithic pharmaceutical dosage form.

In still another preferred embodiment, the total content of the synthetic or natural polymer (C) is within the range of 55±20 wt.-%, more preferably 55±15 wt.-%, and most preferably 55±10 wt.-%, and in particular 55±5 wt.-%, based on the total weight of the monolithic pharmaceutical dosage form.

In yet another preferred embodiment, the total content of the synthetic or natural polymer (C) is within the range of 60±20 wt.-%, more preferably 60±15 wt.-%, and most preferably 60±10 wt.-%, and in particular 60±5 wt.-%, based on the total weight of the monolithic pharmaceutical dosage form.

Preferably, the relative weight ratio of the polymer (C) to the first pharmacologically active ingredient (A₁) is within the range of from 50:1 to 1:20 or 20:1 to 1:20, more preferably 45:1 to 1:15 or 15:1 to 1:15, still more preferably 40:1 to 1:10 or 10:1 to 1:10, yet more preferably 37:1 to 1:7 or 7:1 to 1:7, most preferably 33:1 to 1:5 or 5:1 to 1:5, and in particular 32:1 to 1:2 or 2:1 to 1:2.

The synthetic or natural polymer (C) is preferably selected from the group consisting of polyalkylene oxides (preferably polymethylene oxide, polyethylene oxide, polypropylene oxide), polyalkylenes (preferably polyethylenes, polypropylenes, polyisobutylenes), polyvinyl chlorides, polycarbonates, polystyrenes, polyacrylates, polyacrylic acids, poly(hydroxy fatty acids), poly(hydroxyvaleric acids), polycaprolactones, polyvinyl caprolactames, polyvinyl alcohols, polyesteramides, polyethylene succinates, polylactones, polyglycolides, cellulose ethers (preferably methylcellulose, ethylcellulose, hydroxyethylcellulose, hydroxypropylcellulose, hydroxypropylmethylcellulose), polyurethanes, polyvinylpyrrolidones, polyamides, polylactides, polyacetals, polylactide/glycolides, polylactones, polyglycolides, polyorthoesters, polyanhydrides, copolymers thereof, block-copolymers thereof, and mixtures of at least two of the stated polymers.

In a preferred embodiment, polymer (C) is non-ionic. In another preferred embodiment, polymer (C) is anionic. In still another preferred embodiment, polymer (C) is cationic.

Preferred polyvinyl caprolactames include polyvinyl caprolactam-polyvinyl acetate-polyethylene glycol graft copolymers which are also commercially available as Soluplus®.

Preferably, the synthetic or natural polymer (C) is selected from polyalkylene oxides or acrylic polymers.

In a preferred embodiment,
the content of the synthetic or natural polymer (C) is at least 30 wt.-% relative to the total weight of the segment (S₁) and (S₂), respectively; and/or
the synthetic or natural polymer (C) is selected from polyalkylene oxides or acrylic polymers.

In a particularly preferred embodiment, the segment (S₁) and/or the segment (S₂) contains a pharmacologically active ingredient (A₁) and (A₂), respectively, which is embedded in a matrix material comprising a synthetic or natural polymer (C), wherein
the content of the synthetic or natural polymer (C) is at least 30 wt.-% relative to the total weight of the segment (S₁) and (S₂), respectively; and/or
the synthetic or natural polymer (C) is selected from polyalkylene oxides or acrylic polymers.

In a preferred embodiment, the synthetic or natural polymer (C) is a polyalkylene oxide.

When the prolonged release matrix material of the prolonged release matrix comprises a polyalkylene oxide, it preferably does not additionally comprise an acrylic polymer, a waxy material or a polyalkylene, and vice versa. However, it is principally possible that the prolonged release matrix material of the prolonged release matrix comprises a combination of a polyalkylene oxide, an acrylic polymer, a waxy material and/or a polyalkylene.

In a preferred embodiment, the polyalkylene oxide is homogeneously distributed in the first segment(s) (S₁). According to this embodiment, the first pharmacologically active ingredient (A₁) and the polyalkylene oxide are preferably intimately homogeneously distributed in the first segment(s) (S₁), so that the first segment(s) (S₁) do(es) not contain any portions where either the first pharmacologically active ingredient (A₁) is present in the absence of polyalkylene oxide or where polyalkylene oxide is present in the absence of the first pharmacologically active ingredient (A₁).

When the first segment(s) (S₁) is/are film coated, the polyalkylene oxide is preferably homogeneously distributed in the body of the first segment(s) (S₁), i.e. the film coating preferably does not contain polyalkylene oxide. Nonetheless, the film coating as such may of course contain one or more polymers, which however, preferably differ from the polyalkylene oxide contained in the body.

Preferably, the polyalkylene oxide is selected from polymethylene oxide, polyethylene oxide and polypropylene oxide, or copolymers or mixtures thereof.

Preferably, the polyalkylene oxide has a weight average molecular weight (M_{W}), preferably also a viscosity average molecular weight (M_{η}) of more than 200,000 g/mol or at least 500,000 g/mol, preferably at least 1,000,000 g/mol or at least 2,500,000 g/mol, more preferably in the range of about 1,000,000 g/mol to about 15,000,000 g/mol, and most preferably in the range of about 5,000,000 g/mol to about 10,000,000 g/mol. Suitable methods to determine M_{W} and M_{η} are known to a person skilled in the art. M_{η} is preferably determined by rheological measurements, whereas M_{W} can be determined by gel permeation chromatography (GPC).

Preferably, the molecular weight dispersity M_{w}/Mₙ of the polyalkylene oxide is within the range of 2.5±2.0, more preferably 2.5±1.5, still more preferably 2.5±1.0, yet more preferably 2.5±0.8, most preferably 2.5±0.6, and in particular 2.5±0.4.

The polyalkylene oxide preferably has a viscosity at 25° C. of 30 to 17,600 mPa·s, more preferably 55 to 17,600 mPa·s, still more preferably 600 to 17,600 mPa·s, yet more preferably 4,500 to 17,600 mPa·s, even more preferably 4,500 to 12,000 mPa·s, most preferably 5,000 to 10,500 mPa·s and in particular 5,500 to 7,500 mPa·s or 7,500 to 10,000 mPa·s, measured in a 1 wt.-% aqueous solution.

The polyalkylene oxide may comprise a single polyalkylene oxide having a particular average molecular weight, or a mixture (blend) of different polymers, such as two, three, four or five polymers, e.g., polymers of the same chemical nature but different average molecular weight, polymers of different chemical nature but same average molecular weight, or polymers of different chemical nature as well as different molecular weight.

In a preferred embodiment, the total content of the polyalkylene oxide is at least 20 wt.-%, more preferably at least 15 wt.-%, still more preferably at least 20 wt.-%, most preferably at least 25 wt.-% and in particular at least 30 wt.-%, relative to the total weight of the first segment(s) (S₁).

In a particularly preferred embodiment, the synthetic or natural polymer (C) is a polyalkylene oxide the content of which is at least 30 wt.-% relative to the total weight of the first segment(s) (S₁).

For the purpose of specification, a polyalkylene glycol has a molecular weight of up to 20,000 g/mol whereas a polyalkylene oxide has a molecular weight of more than 20,000 g/mol. The weight average over all molecular weights of all polyalkylene oxides that are contained in the monolithic pharmaceutical dosage form is more than 200,000 g/mol. Thus, polyalkylene glycols, if any, are preferably not taken into consideration when determining the weight average molecular weight of polyalkylene oxide.

In a preferred embodiment, polymer (C) is an acrylic polymer which is preferably derived from a monomer mixture comprising a first C₁₋₄-alkyl(meth)acrylate and a second C₁₋₄-alkyl(meth)acrylate differing from said first C₁₋₄-alkyl(meth)acrylate.

When the prolonged release matrix material of the prolonged release matrix comprises an acrylic polymer, it preferably does not additionally comprise a polyalkylene oxide, a waxy material or a polyalkylene, and vice versa. However, it is principally possible that the prolonged release matrix material of the prolonged release matrix comprises a combination of an acrylic polymer, a polyalkylene oxide, a waxy material and/or a polyalkylene.

Preferred C₁₋₄-alkyl(meth)acrylates include methyl methacrylate, methyl acrylate, ethyl methacrylate, ethyl acrylate, propyl methacrylate, propyl acrylate, butyl methacrylate, and butyl acrylate.

For the purpose of the specification, “(meth)acryl” refers to acryl as well as methacryl.

Preferably, the acrylic polymer has a weight average molecular weight within the range of from 100,000 g/mol to 2,000,000 g/mol. In a preferred embodiment, the acrylic polymer has a weight average molecular weight (M_{W}) or viscosity average molecular weight (M_{η}) of at least 150,000 or at least 200,000 g/mol, preferably at least 250,000 g/mol or at least 300,000 g/mol, more preferably in the range of about 300,000 g/mol to about 2,000,000 g/mol, and most preferably in the range of about 300,000 g/mol to about 1,000,000 g/mol. Suitable methods to determine M_{W} and M_{η} are known to a person skilled in the art. M_{η} is preferably determined by rheological measurements, whereas M_{W} can be determined by gel permeation chromatography (GPC).

The acrylic polymer can be a nonionic acrylic polymer or an ionic acrylic polymer. For the purpose of specification, “nonionic polymer” refers to a polymer not containing more than 1 mole.-% ionic, i.e. anionic or cationic, monomer units, preferably containing no ionic monomer units at all.

In a preferred embodiment, the synthetic or natural polymer (C) is a nonionic acrylic polymer which is preferably derived from a monomer mixture comprising a first C₁₋₄-alkyl(meth)acrylate and a second C₁₋₄-alkyl(meth)acrylate differing from said first C₁₋₄-alkyl(meth)acrylate.

Preferably, the first C₁₋₄-alkyl(meth)acrylate is ethyl acrylate and the second C₁₋₄-alkyl(meth)acrylate is methyl methacrylate.

Preferably, the relative molar content of the ethyl acrylate within the nonionic acrylic polymer is greater than the relative molar content of the methyl methacrylate within the nonionic acrylic polymer.

Preferably, the molar ratio of the first C₁₋₄-alkyl(meth)acrylate, which is preferably ethyl acrylate, to the second C₁₋₄-alkyl(meth)acrylate, which is preferably methyl methacrylate, is within the range of from 5:1 to 1:3, more preferably from 4.5:1 to 1:2.5, still more preferably from 4:1 to 1:2, yet more preferably from 3.5:1 to 1:1.5, even more preferably from 3:1 to 1:1, most preferably from 2.5:1 to 1.5:1, and in particular about 2:1.

Preferably, the nonionic acrylic polymer has a weight average molecular weight within the range of from 100,000 g/mol to 2,000,000 g/mol. In a preferred embodiment, the nonionic acrylic polymer has a weight average molecular weight (M_{W}) or viscosity average molecular weight (M_{η}) of at least 150,000 or at least 200,000 g/mol, preferably at least 250,000 g/mol or at least 300,000 g/mol, more preferably in the range of about 300,000 g/mol to about 2,000,000 g/mol, and most preferably in the range of about 300,000 g/mol to about 1,500,000 g/mol. Suitable methods to determine M_{W} and M_{η} are known to a person skilled in the art. M_{η} is preferably determined by rheological measurements, whereas M_{W} can be determined by gel permeation chromatography (GPC).

In a preferred embodiment, the weight average molecular weight of the nonionic acrylic polymer is within the range of 675,000±500,000 g/mol, more preferably 675,000±450,000 g/mol, still more preferably 675,000±400,000 g/mol, yet more preferably 675,000±350,000 g/mol, even more preferably 675,000±300,000 g/mol, most preferably 675,000±250,000 g/mol, and in particular 675,000±200,000 g/mol.

The nonionic acrylic polymer may comprise a single nonionic acrylic polymer having a particular average molecular weight, or a mixture (blend) of different nonionic acrylic polymers, such as two, three, four or five nonionic acrylic polymers, e.g., nonionic acrylic polymers of the same chemical nature but different average molecular weight, nonionic acrylic polymers of different chemical nature but same average molecular weight, or nonionic acrylic polymers of different chemical nature as well as different molecular weight.

In a preferred embodiment, the nonionic acrylic polymer is homogeneously distributed in the first segment(s) (S₁). According to this embodiment, the first pharmacologically active ingredient (A₁) and the nonionic acrylic polymer preferably are intimately homogeneously distributed in the first segment(s) (S₁), so that the first segment(s) (S₁) do(es) not contain any portions where either the first pharmacologically active ingredient (A₁) is present in the absence of nonionic acrylic polymer or where nonionic acrylic polymer is present in the absence of the first pharmacologically active ingredient (A₁).

When the first segment(s) (S₁) is/are film coated, the nonionic acrylic polymer is preferably homogeneously distributed in the body of the first segment(s) (S₁), i.e. the film coating preferably does not contain nonionic acrylic polymer. Nonetheless, the film coating as such may of course contain one or more polymers, which however, preferably differ from the nonionic acrylic polymer contained in the body.

The nonionic acrylic polymer preferably has a glass transition temperature (T_{g}) within the range of 1±15° C., more preferably 1±11° C.

The nonionic acrylic polymer preferably has a minimum film forming temperature (MFT) within the range of 5±5° C., more preferably 5±2° C.

Nonionic acrylic polymers that are suitable for use in the first segment (S₁) according to the invention are commercially available, e.g. from Evonik. For example, Eudragit® NE30D, Eudragit® NE40D and Eudragit® NM30D, which are provided as aqueous dispersions of poly(ethyl acrylate-co-methyl methacrylate) 2:1, may be used in the first segment (S₁) according to the invention. For details concerning the properties of these products, it can be referred to e.g. the product specification.

In a preferred embodiment, the synthetic or natural polymer (C) is an ionic acrylic polymer.

In a preferred embodiment, the ionic acrylic polymer is homogeneously distributed in the first segment(s) (S₁). According to this embodiment, the first pharmacologically active ingredient (A₁) and the ionic acrylic polymer preferably are intimately homogeneously distributed in the first segment(s) (S₁), so that the first segment(s) (S₁) do(es) not contain any portions where either the first pharmacologically active ingredient (A₁) is present in the absence of ionic acrylic polymer or where ionic acrylic polymer is present in the absence of the first pharmacologically active ingredient (A₁).

When the first segment(s) (S₁) is/are film coated, the ionic acrylic polymer is preferably homogeneously distributed in the body of the first segment(s) (S₁), i.e. the film coating preferably does not contain ionic acrylic polymer. Nonetheless, the film coating as such may of course contain one or more polymers, which however, preferably differ from the ionic acrylic polymer contained in the body.

Preferred ionic acrylic polymers are anionic acrylic polymers. Preferred anionic acrylic polymers include but are not limited to copolymers of one or two different C₁₋₄-alkyl(meth)acrylate monomers and copolymerizable anionic monomers such as acrylic acid. Preferred representatives are ternary copolymers of methyl acrylate, methyl methacrylate and methacrylic acid, wherein the relative molar content of the monomers is preferably methyl acrylate>methyl methacrylate>methacrylic acid. In a preferred embodiment, the anionic acrylic polymer has a weight average molecular weight within the range of 125,000±100,000 g/mol, more preferably 125,000±90,000 g/mol, still more preferably 125,000±80,000 g/mol, yet more preferably 125,000±70,000 g/mol, even more preferably 125,000±60,000 g/mol, most preferably 125,000±50,000 g/mol, and in particular 125,000±40,000 g/mol. Poly(methacrylic acid-co-methyl methacrylate) 1:2 having an average molecular weight of about 125,000 g/mol is commercially available as Eudragit® FS 100. In another preferred embodiment, the anionic acrylic polymer has a weight average molecular weight within the range of 280,000±250,000 g/mol, more preferably 280,000±200,000 g/mol, still more preferably 280,000±180,000 g/mol, yet more preferably 280,000±160,000 g/mol, even more preferably 280,000±140,000 g/mol, most preferably 280,000±120,000 g/mol, and in particular 280,000±100,000 g/mol. Poly(methyl acrylate-co-methyl methacrylate-co-methacrylic acid) 7:3:1 having an average molecular weight of about 280,000 g/mol is commercially available as Eudragit® FS. In still another preferred embodiment, the anionic acrylic polymer has a weight average molecular weight within the range of 1,250,000±1,000,000 g/mol, more preferably 1,250,000±900,000 g/mol, still more preferably 1,250,000±800,000 g/mol, yet more preferably 1,250,000±700,000 g/mol, most preferably 1,250,000±600,000 g/mol, and in particular 1,250,000±500,000 g/mol. According to this embodiment, the anionic acrylic polymer preferably is polyacrylic acid which is optionally crosslinked, preferably with allyl ethers of pentaerythritol. Polyacrylic acid or carbomer homopolymer is commercially available as Carbopol® 71 G.

Other preferred ionic acrylic polymers are cationic acrylic polymers. Preferred cationic acrylic polymers include but are not limited to copolymers of one or two different C₁₋₄-alkyl(meth)acrylate monomers and copolymerizable cationic monomers such as trimethylammonioethyl methacrylate chloride. Preferred representatives are ternary copolymers of ethyl acrylate, methyl methacrylate and a low content of methacrylic acid ester with quaternary ammonium groups, preferably trimethylammonioethyl methacrylate chloride, wherein the relative molar content of the monomers is preferably methyl methacrylate>ethyl acrylate>copolymerizable cationic monomers. Preferably, the cationic acrylic polymer has a weight average molecular weight within the range of 32,000±30,000 g/mol, more preferably 32,000±27,000 g/mol, still more preferably 32,000±23,000 g/mol, yet more preferably 32,000±20,000 g/mol, even more preferably 32,000±17,000 g/mol, most preferably 32,000±13,000 g/mol, and in particular 32,000±10,000 g/mol. Poly(ethyl acrylate-co-methyl methacrylate-co-trimethylammonioethyl methacrylate chloride) 1:2:0.1 and 1:2:0.2, respectively, having an average molecular weight of about 32,000 g/mol is commercially available as Eudragit® RS-PO and Eudragit® RL-PO, respectively. Because of its lower content of trimethylammonioethyl methacrylate chloride, Eudragit® RS-PO is particularly preferred. Another preferred cationic acrylic polymer is Eudragit® RL 100 which is a copolymer of ethyl acrylate, methyl methacrylate and a low content of methacrylic acid ester with quaternary ammonium groups.

In another preferred embodiment, the synthetic or natural polymer (C) is a polyalkylene.

When the prolonged release matrix material of the prolonged release matrix comprises a polyalkylene, it preferably does not additionally comprise a polyalkylene oxide, an acrylic polymer or a waxy material, and vice versa. However, it is principally possible that the prolonged release matrix material of the prolonged release matrix comprises a combination of a polyalkylene, a polyalkylene oxide, an acrylic polymer and/or a waxy material.

In a preferred embodiment, the polyalkylene is homogeneously distributed in the first segment(s) (S₁). According to this embodiment, the first pharmacologically active ingredient (A₁) and the polyalkylene preferably are intimately homogeneously distributed in the first segment(s) (S₁), so that the first segment(s) (S₁) do(es) not contain any portions where either the first pharmacologically active ingredient (A₁) is present in the absence of polyalkylene or where polyalkylene is present in the absence of the first pharmacologically active ingredient (A₁).

When the first segment(s) (S₁) is/are film coated, the polyalkylene is preferably homogeneously distributed in the body of the first segment(s) (S₁), i.e. the film coating preferably does not contain polyalkylene. Nonetheless, the film coating as such may of course contain one or more polymers, which however, preferably differ from the polyalkylene contained in the body.

Preferably, the polyalkylene is selected from polyethylenes, polypropylenes, polyisobutylenes or copolymers or mixtures thereof.

Preferably, the polyalkylene has a weight average molecular weight (M_{W}), preferably also a viscosity average molecular weight (M_{η}) of at least 10,000 g/mol, preferably at least 20,000 g/mol, more preferably in the range of about 20,000 g/mol to about 1,000,000 g/mol, and most preferably in the range of about 30,000 g/mol to about 100,000 g/mol.

Preferably, the molecular weight dispersity M_{w}/Mₙ of the polyalkylene is within the range of 3.2±2.0, more preferably 3.2±1.5, still more preferably 3.2±1.0, yet more preferably 3.2±0.8, most preferably 3.2±0.6, and in particular 3.2±0.4.

The polyalkylene preferably has a Brookfield viscosity at 150° C. of 10,000 to 1,000,000 mPa·s, more preferably 15,000 to 950,000 mPa·s, still more preferably 20,000 to 900,000 mPa·s, yet more preferably 23,000 to 850,000 mPa·s, even more preferably 25,000 to 800,000 mPa·s, most preferably 28,000 to 750,000 mPa·s and in particular 30,000 to 710,000 mPa·s.

The polyalkylene may comprise a single polyalkylene having a particular average molecular weight, or a mixture (blend) of different polymers, such as two, three, four or five polymers, e.g., polymers of the same chemical nature but different average molecular weight, polymers of different chemical nature but same average molecular weight, or polymers of different chemical nature as well as different molecular weight.

In a preferred embodiment, the synthetic or natural polymer (C) is polyisobutylene, preferably having a weight average molecular weight M_{w} of 36,000±1,000 g/mol.

Preferred polyisobutylenes include Oppanol® B 10, Oppanol® B 11, Oppanol® B 12, Oppanol® B 13, Oppanol® B 14 and Oppanol® B 15.

In another preferred embodiment, the prolonged release matrix material comprises a waxy material, preferably selected from the group consisting of
glycerides, especially monoglycerides, diglycerides, triglycerides,
esters of fatty acids with fatty alcohols, and
paraffins.

When the prolonged release matrix material of the prolonged release matrix comprises a waxy material, it preferably does not additionally comprise an acrylic polymer or a polyalkylene oxide, and vice versa.

As used herein a “waxy material” refers to a material which melts into liquid form having low viscosity upon heating and sets again to a solid state upon cooling. Preferably, the waxy material has a melting point of at least 30° C., more preferably at least 35° C., still more preferably at least 40° C., yet more preferably at least 45° C., even more preferably at least 50° C., most preferably at least 55° C., and in particular at least 60° C.

When the waxy material is or comprises a monoglyceride, diglyceride, triglyceride or a mixture thereof, it is preferably a mono-, di- or triester of glycerol and carboxylic acids, whereas the carboxylic acid is preferably selected from the group consisting of fatty acids, hydroxy fatty acids and aromatic acids.

In another preferred embodiment, the glyceride is a fatty acid macrogolglyceride, e.g. lauroyl macrogolglyceride, such as Gelucire 44/14 that can be regarded as a non-ionic water dispersible surfactant composed of well-characterized PEG-esters, a small glyceride fraction and free PEG

Preferred glycerides of fatty acids include monoglycerides, diglycerides, triglycerides, and mixtures thereof; preferably of C₆ to C₂₂ fatty acids. Especially preferred are partial glycerides of the C₁₆ to C₂₂ fatty acids such as glycerol behenate, glycerol monostearate, glycerol palmitostearate and glyceryl distearate as well as triglycerides of the C₁₆ to C₂₂ fatty acids such as glycerol tristearate.

The term “fatty acid” is well acknowledged in the art and includes for example unsaturated representatives such as myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, and docosahexaenoic acid; as well as saturated representatives such as caprylic acid, capric acid, lauric acid, myristic acid, palmitic acid, stearic acid, arachidic acid, behenic acid, lignoceric acid, and cerotic acid.

The term “hydroxy fatty acid” is also well acknowledged in the art and includes for example 2-hydroxyhexanoic acid, 2-hydroxyoctanoic acid, 2-hydroxydecanoic acid, 2-hydroxydodecanoic acid, β-hydroxylauric acid, 2-hydroxytetradecanoic acid, β-hydroxymyristic acid, 15-hydroxypentadecanoic acid, 16-hydroxyhexadecanoic acid, β-hydroxypalmitic acid, 12-hydroxyoctadecanoic acid, α-hydroxystearic acid, and α-hydroxyarachidic acid.

The fatty acids and the hydroxy fatty acids are preferably saturated.

When the waxy material is or comprises a diglyceride or a triglyceride, the fatty acids, hydroxy fatty acids and aromatic acids, respectively, may be identical or different.

According to this embodiment of the invention, the waxy material is preferably a hard fat (adeps solidus) in accordance with Ph. Eur.

Preferably, the waxy material is a monoglyceride, diglyceride, triglyceride or a mixture thereof, selected from the group consisting of hydrogenated soybean oil, hydrogenated palm oil, hydrogenated castor oil, hydrogenated cottonseed oil, and mixtures thereof.

When the waxy material is or comprises an ester of a fatty acid with a fatty alcohol, the fatty acid is preferably a saturated fatty acid. Preferred examples of fatty acids are already mentioned above in connection with the glycerides. The fatty alcohol is preferably derived from a fatty acid and preferably also saturated.

Preferred representatives of esters of fatty acids with fatty alcohols include but are not limited to natural waxes such as beeswax, carnaubawax, candelilla wax, ouricury wax, sugarcane wax, cetyl palmitate, oleyl oleate, cetaceum and retamo wax.

When the waxy material is or comprises paraffin, the paraffin is preferably a hard paraffin (paraffinum solidum, ceresin, zeresin) in accordance with Ph. Eur.

The waxy material may comprise a single waxy material, or a mixture (blend) of different waxy materials, such as two, three, four or five waxy materials, each of which preferably being selected from the group consisting of glycerides, especially monoglycerides, diglycerides, triglycerides; esters of fatty acids with fatty alcohols; and paraffins.

In a preferred embodiment, the waxy material is homogeneously distributed in the first segment(s) (S₁). According to this embodiment, the first pharmacologically active ingredient (A₁) and the waxy material preferably are intimately homogeneously distributed in the first segment(s) (S₁), so that the first segment(s) (S₁) do(es) not contain any portions where either the first pharmacologically active ingredient (A₁) is present in the absence of waxy material or where waxy material is present in the absence of the first pharmacologically active ingredient (A₁).

When the first segment(s) (S₁) is/are film coated, the waxy material is preferably homogeneously distributed in the first segment(s) (S₁), i.e. the film coating preferably does not contain waxy material. Nonetheless, the film coating as such may of course contain one or more waxy materials, which however, preferably differ from the waxy materials contained in the body.

Waxy materials that are suitable for use in the pharmaceutical dosage forms according to the invention are commercially available, e.g. Cera alba, Cera flava, Kolliwax™ HCO, Dynasan® 118, Compritol® 888 ATO, Precirol® ATO 5, Gelucire® 44/14, and the like. For details concerning the properties of these products, it can be referred to e.g. the product specification.

The total content of the waxy material is preferably within the range of from 5.0 to 95 wt.-%, more preferably 7 to 90 wt.-%, still more preferably 9 to 85 wt.-%, yet more preferably 11 to 80 wt.-%, most preferably 13 to 75 wt.-%, and in particular 15 to 70 wt.-%, relative to the total weight of the prolonged release matrix.

Preferably, the total content of the waxy material is within the range of from 1 to 90 wt.-%, more preferably 3 to 85 wt.-%, still more preferably 5 to 80 wt.-%, yet more preferably 7 to 75 wt.-%, most preferably 10 to 70 wt.-% and in particular 15 to 65 wt.-%, based on the total weight of the first segment(s) (S₁).

In a preferred embodiment, the total content of the waxy material is at least 2 wt.-%, more preferably at least 5 wt.-%, still more preferably at least 10 wt.-%, yet more preferably at least 15 wt.-% and in particular at least 20 wt.-%, based on the total weight of the first segment(s) (S₁).

In a preferred embodiment, the total content of waxy material is within the range of 10±8 wt.-%, more preferably 10±6 wt.-%, most preferably 10±4 wt.-%, and in particular 10±2 wt.-%, based on the total weight of the first segment(s) (S₁).

In another preferred embodiment, the total content of waxy material is within the range of 15±12 wt.-%, more preferably 15±10 wt.-%, most preferably 15±7 wt.-%, and in particular 15±3 wt.-%, based on the total weight of the first segment(s) (S₁).

In still another preferred embodiment, the total content of waxy material is within the range of 20±16 wt.-%, more preferably 20±12 wt.-%, most preferably 20±8 wt.-%, and in particular 20±4 wt.-%, based on the total weight of the first segment(s) (S₁).

In yet another preferred embodiment, the total content of waxy material is within the range of 25±20 wt.-%, more preferably 25±15 wt.-%, most preferably 25±10 wt.-%, and in particular 25±5 wt.-%, based on the total weight of the first segment(s) (S₁).

In a further preferred embodiment, the total content of waxy material is within the range of 30±20 wt.-%, more preferably 30±15 wt.-%, most preferably 30±10 wt.-%, and in particular 30±5 wt.-%, based on the total weight of the first segment(s) (S₁).

In still a further preferred embodiment, the total content of waxy material is within the range of 35±20 wt.-%, more preferably 35±15 wt.-%, most preferably 35±10 wt.-%, and in particular 35±5 wt.-%, based on the total weight of the first segment(s) (S₁).

In a still further preferred embodiment, the total content of waxy material is within the range of 40±20 wt.-%, more preferably 40±15 wt.-%, and most preferably 40±10 wt.-%, and in particular 40±5 wt.-%, based on the total weight of the first segment(s) (S₁).

In a yet further preferred embodiment, the total content of waxy material is within the range of 45±20 wt.-%, more preferably 45±15 wt.-%, and most preferably 45±10 wt.-%, and in particular 45±5 wt.-%, based on the total weight of the first segment(s) (S₁).

In another preferred embodiment, the total content of waxy material is within the range of 50±20 wt.-%, more preferably 50±15 wt.-%, and most preferably 50±10 wt.-%, and in particular 50±5 wt.-%, based on the total weight of the first segment(s) (S₁).

In a yet further preferred embodiment, the total content of waxy material is within the range of 55±20 wt.-%, more preferably 55±15 wt.-%, and most preferably 55±10 wt.-%, and in particular 55±5 wt.-%, based on the total weight of the first segment(s) (S₁).

In another preferred embodiment, the total content of waxy material is within the range of 60±20 wt.-%, more preferably 60±15 wt.-%, and most preferably 60±10 wt.-%, and in particular 60±5 wt.-%, based on the total weight of the first segment(s) (S₁).

In a yet further preferred embodiment, the total content of waxy material is within the range of 65±20 wt.-%, more preferably 65±15 wt.-%, and most preferably 65±10 wt.-%, and in particular 65±5 wt.-%, based on the total weight of the first segment(s) (S₁).

In another preferred embodiment, the total content of waxy material is within the range of 70±20 wt.-%, more preferably 70±15 wt.-%, and most preferably 70±10 wt.-%, and in particular 70±5 wt.-%, based on the total weight of the first segment(s) (S₁).

In a yet further preferred embodiment, the total content of waxy material is within the range of 75±20 wt.-%, more preferably 75±15 wt.-%, and most preferably 75±10 wt.-%, and in particular 75±5 wt.-%, based on the total weight of the first segment(s) (S₁).

In another preferred embodiment, the total content of waxy material is within the range of 80±20 wt.-%, more preferably 80±15 wt.-%, and most preferably 80±10 wt.-%, and in particular 80±5 wt.-%, based on the total weight of the first segment(s) (S₁).

Preferably, the relative weight ratio of the waxy material to the first pharmacologically active ingredient (A₁) is within the range of 20:1 to 1:20, more preferably 15:1 to 1:15, still more preferably 10:1 to 1:10, yet more preferably 7:1 to 1:7, most preferably 5:1 to 1:5, and in particular 2:1 to 1:2 or 1:1 to 1:3.

Besides the preferably present first pharmacologically active ingredient (A₁) and the preferably present prolonged release matrix material the first segment(s) (S₁) may optionally further comprise additional pharmaceutical excipients conventionally contained in pharmaceutical dosage forms in conventional amounts, such as antioxidants, preservatives, lubricants, plasticizer, fillers/binders, and the like.

The skilled person will readily be able to determine appropriate further excipients as well as the quantities of each of these excipients. Specific examples of pharmaceutically acceptable carriers and excipients are described in the Handbook of Pharmaceutical Excipients, American Pharmaceutical Association (1986).

In a preferred embodiment, when the first segment(s) (S₁) provide(s) prolonged release of the first pharmacologically active ingredient (A₁), the first segment(s) (S₁) do(es) not contain a disintegrant.

Preferably, when the first segment(s) (S₁) provide(s) prolonged release of the first pharmacologically active ingredient (A₁), the first segment(s) (S₁) further comprise(s) an antioxidant. Suitable antioxidants include ascorbic acid, butylated hydroxyanisole (BHA), butylated hydroxytoluene (BHT), salts of ascorbic acid, monothioglycerol, phosphorous acid, vitamin C, vitamin E and the derivatives thereof, coniferyl benzoate, nordihydroguajaretic acid, gallus acid esters, sodium bisulfite, particularly preferably butylhydroxytoluene or butylhydroxyanisole and α-tocopherol. The antioxidant is preferably present in quantities of 0.005 wt.-% to 10 wt.-%, more preferably of 0.01 wt.-% to 8 wt.-%, most preferably of 0.04 wt.-% to 6 wt.-%, based on the total weight of the first segment(s) (S₁).

In a preferred embodiment, when the first segment(s) (S₁) provide(s) prolonged release of the first pharmacologically active ingredient (A₁), the first segment(s) (S₁) further comprise(s) an acid, preferably a carboxylic acid, more preferably a multicarboxylic acid, particularly citric acid. The amount of acid is preferably in the range of 0.01 wt.-% to about 20 wt.-%, more preferably in the range of 0.02 wt.-% to about 10 wt.-%, and still more preferably in the range of 0.05 wt.-% to about 5 wt.-%, and most preferably in the range of 0.1 wt.-% to about 1.0 wt.-%, based on the total weight of the first segment(s) (S₁).

In a preferred embodiment, when the first segment(s) (S₁) provide(s) prolonged release of the first pharmacologically active ingredient (A₁), the first segment(s) (S₁) contain(s) at least one lubricant. In another preferred embodiment, the first segment(s) (S₁) contain(s) no lubricant.

Especially preferred lubricants are selected from
magnesium stearate, calcium stearate and stearic acid;
polyoxyethylene glycerol fatty acid esters, such as mixtures of mono-, di- and triesters of glycerol and di- and monoesters of macrogols having molecular weights within the range of from 200 to 4000 g/mol, e.g., macrogolglycerolcaprylocaprate, macrogolglycerollaurate, macrogolglycerolococoate, macrogolglycerollinoleate, macrogol-20-glycerolmonostearate, macrogol-6-glycerolcaprylocaprate, macrogolglycerololeate; macrogolglycerolstearate, macrogolglycerolhydroxystearate, and macrogolglycerolrizinoleate;
polyglycolyzed glycerides, such as the one known and commercially available under the trade name “Labrasol”;
fatty alcohols that may be linear or branched, such as cetylalcohol, stearylalcohol, cetylstearyl alcohol, 2-octyldodecane-1-ol and 2-hexyldecane-1-ol; and
polyethylene glycols having a molecular weight between 10.000 and 60.000 g/mol.

Particularly preferred lubricants comprise stearyl alcohol, stearic acid and calcium stearate or a mixture thereof.

Preferably, the amount of the lubricant ranges from 0.01 wt.-% to about 10 or 15 wt.-%, more preferably in the range of 0.05 wt.-% to about 10 wt.-%, most preferably in the range of 0.1 wt.-% to about 5 wt.-% or 1.5 wt.-% to about 8 wt.-%, and in particular in the range of 0.1 wt.-% to about 1 wt.-% or 3 to about 7 wt.-%, based on the total weight of the first segment(s) (S₁).

When the first segment(s) (S₁) contain(s) more than one lubricant, preferably, the overall amount of the lubricant ranges from 1 wt.-% to about 20 wt.-%, more preferably in the range of 5 wt.-% to about 18 wt.-%, most preferably in the range of 7 wt.-% to about 15 wt.-%, and in particular in the range of 8 wt.-% to about 12 wt.-%, based on the total weight of the first segment(s) (S₁).

Preferably, when the first segment(s) (S₁) provide(s) prolonged release of the first pharmacologically active ingredient (A₁), the first segment(s) (S₁) further comprise(s) a plasticizer. The plasticizer improves the processability of the prolonged release matrix material. A preferred plasticizer is polyalkylene glycol, like polyethylene glycol, triethyl citrate (TEC), triacetin, fatty acids, fatty acid esters, waxes and/or microcrystalline waxes. Particularly preferred plasticizers are polyethylene glycols, such as PEG 6000. Further particularly preferred plasticizers comprise triethyl citrate (TEC), stearic acid, calcium stearate and stearyl alcohol or a mixture thereof.

Preferably, the content of the plasticizer is within the range of from 0.5 to 30 wt.-%, more preferably 1 to 25 wt.-%, still more preferably 2 wt.-% to 22 wt.-%, yet more preferably 5 wt.-% to 21 wt.-%, most preferably 7 to 20 wt.-% and in particular 8 wt.-% to 19 wt.-%, based on the total weight of the first segment(s) (S₁).

When the first segment (S₁) contains more than one plasticizer, preferably, the overall amount of the plasticizer ranges from 3 wt.-% to about 30 wt.-%, more preferably in the range of 5 wt.-% to about 25 wt.-%, most preferably in the range of 7 wt.-% to about 15 wt.-%, and in particular in the range of 8 wt.-% to about 20 wt.-%, based on the total weight of the first segment(s) (S₁).

Plasticizers can sometimes act as a lubricant, and lubricants can sometimes act as a plasticizer.

Preferably, when the first segment(s) (S₁) provide(s) prolonged release of the first pharmacologically active ingredient (A₁), the first segment(s) (S₁) further comprise(s) a filler/binder. A preferred filler/binder is selected from celluloses, cellulose derivatives such as cellulose ethers and cellulose esters, tricalcium phosphate, poloxamers (e.g. Lutrol® F68) and isomalt. A particularly preferred filler/binder is selected from cellulose esters and cellulose ethers, in particular hydroxypropyl methylcellulose (HPMC).

The amount of the filler/binder, preferably HPMC, preferably ranges from 0.1 wt.-% to about 30 wt.-%, more preferably in the range of 1.0 wt.-% to about 20 wt.-%, and most preferably in the range of 2.0 wt.-% to about 18 wt.-% relative to the total weight of the first segment(s) (S₁).

In a preferred embodiment, besides the preferably present first pharmacologically active ingredient (A₁) that may have any solubility in aqueous ethanol, relative to the total weight of the first segment(s) (S₁), the first segment(s) (S₁) according to the invention preferably contain(s) at most 25 wt.-%, more preferably at most 20 wt.-%, still more preferably at most 15 wt.-%, yet more preferably at most 10 wt.-%, even more preferably at most 5.0 wt.-%, most preferably at most 2.5 wt.-%, and in particular at most 1.0 wt.-% of ingredients (prolonged release matrix material, excipients, and the like) having at room temperature in aqueous ethanol (40 vol.-%) a solubility of at least 100 mg/ml, more preferably a solubility of at least 75 mg/ml, still more preferably a solubility of at least 50 mg/ml, yet more preferably a solubility of at least 25 mg/ml, even more preferably a solubility of at least 10 mg/ml, most preferably a solubility of at least 5.0 mg/ml, and in particular a solubility of at least 1.0 mg/ml.

Preferred contents of the first pharmacologically active ingredient (A₁), prolonged release matrix material, and excipients, relative to the total weight of the first segment(s) (S₁), are summarized as embodiments B¹⁷ to B⁴⁵ in the tables here below:

| | wt.-% | | | |
|---|---|---|---|---|
| | B¹⁷ | B¹⁸ | B¹⁹ | B²⁰ |
| first pharmacologically active | 40 ± 30 | 40 ± 20 | 40 ± 10 | 40 ± 5 |
| ingredient (A₁) | | | | |
| synthetic or natural polymer (C) | 50 ± 30 | 50 ± 20 | 50 ± 10 | 50 ± 5 |
| pharmaceutical excipients | 20 ± 20 | 20 ± 20 | 20 ± 20 | 20 ± 20 |

| | wt.-% | | | |
|---|---|---|---|---|
| | B²¹ | B²² | B²³ | B²⁴ |
| first pharmacologically active | 30 ± 25 | 30 ± 20 | 30 ± 10 | 30 ± 5 |
| ingredient (A₁) | | | | |
| synthetic or natural polymer (C) | 50 ± 30 | 50 ± 20 | 50 ± 10 | 50 ± 5 |
| pharmaceutical excipients | 20 ± 20 | 20 ± 20 | 20 ± 20 | 20 ± 20 |

| | wt.-% | | | |
|---|---|---|---|---|
| | B²⁵ | B²⁶ | B²⁷ | B²⁸ |
| first pharmacologically active | 20 ± 15 | 20 ± 12.5 | 20 ± 10 | 20 ± 5 |
| ingredient (A₁) | | | | |
| synthetic or natural polymer (C) | 60 ± 30 | 60 ± 20 | 60 ± 10 | 60 ± 5 |
| pharmaceutical excipients | 20 ± 20 | 20 ± 20 | 20 ± 20 | 20 ± 20 |

| | wt.-% | | | |
|---|---|---|---|---|
| | B²⁹ | B³⁰ | B³¹ | B³² |
| first pharmacologically active | 20 ± 15 | 20 ± 12.5 | 20 ± 10 | 20 ± 5 |
| ingredient (A₁) | | | | |
| synthetic or natural polymer (C) | 50 ± 30 | 50 ± 20 | 50 ± 10 | 50 ± 5 |
| pharmaceutical excipients | 20 ± 20 | 20 ± 20 | 20 ± 20 | 20 ± 20 |

| wt.-% | B³³ | B³⁴ | B³⁵ | B³⁶ |
|---|---|---|---|---|
| first pharmacologically | 20 ± 15 | 20 ± 12.5 | 20 ± 10 | 20 ± 5 |
| active ingredient (A₁) | | | | |
| synthetic or natural | 40 ± 30 | 40 ± 20 | 40 ± 10 | 40 ± 5 |
| polymer (C) | | | | |
| pharmaceutical excipients | 20 ± 20 | 20 ± 20 | 20 ± 20 | 20 ± 20 |

| wt.-% | B³⁷ | B³⁸ | B³⁹ | B⁴⁰ |
|---|---|---|---|---|
| first pharmacologically | 10 ± 7.5 | 10 ± 7.5 | 10 ± 5 | 10 ± 5 |
| active ingredient (A₁) | | | | |
| synthetic or natural | 50 ± 30 | 50 ± 20 | 50 ± 10 | 50 ± 5 |
| polymer (C) | | | | |
| pharmaceutical excipients | 20 ± 20 | 20 ± 20 | 20 ± 20 | 20 ± 20 |

| wt.-% | B⁴¹ | B⁴² | B⁴³ | B⁴⁴ |
|---|---|---|---|---|
| first pharmacologically active | 5 ± 4 | 5 ± 4 | 5 ± 3 | 5 ± 3 |
| ingredient (A₁) | | | | |
| synthetic or natural polymer (C) | 70 ± 20 | 70 ± 15 | 70 ± 10 | 70 ± 5 |
| pharmaceutical excipients | 20 ± 20 | 20 ± 20 | 20 ± 20 | 20 ± 20 |

Preferably, the first segment(s) (S₁) provide(s) prolonged release of the first pharmacologically active ingredient (A₁). Preferably, the prolonged release matrix provides for a prolonged release of the first pharmacologically active ingredient (A₁) from the first segment (S₁).

Preferably, under in vitro conditions the monolithic pharmaceutical dosage form has released after 30 minutes 0.1 to 75%, after 240 minutes 0.5 to 99%, after 480 minutes 1.0 to 100% and after 720 minutes 2.5 to 100% of the first pharmacologically active ingredient (A₁).

Suitable in vitro conditions are known to the skilled artisan. In this regard it can be referred to, e.g., the Eur. Ph. Preferably, the release profile is measured under the following conditions: Paddle apparatus, 50 rpm, 37±5° C., 900 mL 0.1 M HCl (pH 1.0) or simulated intestinal fluid pH 6.8 (phosphate buffer) or pH 4.5. In another preferred embodiment, the rotational speed of the paddle is increased to 75 rpm. In another preferred embodiment, the release profile is determined under the following conditions: basket method, 75 rpm, 37±5° C., 900 mL 0.1 N HCl or 900 mL of SIF sp (pH 6.8) or 900 mL of 0.1 N HCl+40% ethanol.

Preferred release profiles R¹⁰ to R¹⁷ are summarized in the table here below [all data in wt.-% of released first pharmacologically active ingredient (A₁)]:

| time | R¹⁰ | R¹¹ | R¹² | R¹³ | R¹⁴ | R¹⁵ | R¹⁶ | R¹⁷ |
|---|---|---|---|---|---|---|---|---|
| 60 min | 0-60 | 0-10 | 2-20 | 4-20 | 5-30 | 15-40 | 15-50 | 20-65 |
| 120 min | 0-90 | 1-60 | 5-30 | 10-35 | 10-35 | 20-55 | 25-80 | 30-90 |
| 240 min | 1-99 | 5-95 | 15-45 | 25-85 | 15-45 | 40-80 | 35-100 | 50-95 |
| 480 min | 5-100 | 7-100 | 25-85 | 60-100 | 20-60 | 60-100 | 45-100 | 70-100 |
| 720 min | 10-100 | 10-100 | 35-100 | 80-100 | 30-80 | >80 | >80 | 70-100 |
| 960 min | 20-100 | 15-100 | 50-100 | >90 | 40-90 | >90 | >90 | >80 |
| 1440 min | 50-100 | 30-100 | 60-100 | >99 | >60 | >99 | >99 | >90 |
| 2160 min | >80 | >80 | >80 | | >80 | | | >99 |

Further preferred release profiles R¹⁸ to R²⁴ are summarized in the table here below [all data in wt.-% of released first pharmacologically active ingredient (A₁)]:

| time | R¹⁸ | R¹⁹ | R²⁰ | R²¹ | R²² | R²³ | R²⁴ |
|---|---|---|---|---|---|---|---|
| 15 min | 3 ± 3 | 2 ± 2 | 11 ± 5 | 1 ± 1 | 14 ± 5 | 6 ± 4 | 2 ± 2 |
| 30 min | 12 ± 6 | 4 ± 3 | 16 ± 5 | 1 ± 1 | 23 ± 8 | 9 ± 4 | 5 ± 3 |
| 45 min | 25 ± 10 | 6 ± 5 | 21 ± 5 | 1 ± 1 | 33 ± 8 | 12 ± 5 | 7 ± 5 |
| 60 min | 32 ± 10 | 7 ± 5 | 25 ± 10 | 1 ± 1 | 45 ± 10 | 14 ± 5 | 9 ± 5 |
| 120 min | 60 ± 12 | 13 ± 5 | 39 ± 10 | 46 ± 10 | 70 ± 20 | 21 ± 8 | 22 ± 10 |
| 240 min | 88 ± 10 | 29 ± 10 | 61 ± 10 | 84 ± 16 | 86 ± 14 | 31 ± 8 | 56 ± 25 |
| 480 min | 95 ± 5 | 58 ± 25 | 86 ± 15 | 99 ± 1 | 92 ± 8 | 46 ± 8 | 94 ± 6 |
| 720 min | 99 ± 1 | 78 ± 22 | 90 ± 10 | | 92 ± 8 | 57 ± 8 | 95 ± 5 |
| 735 min | | 80 ± 20 | 91 ± 9 | | 92 ± 8 | 59 ± 15 | 99 ± 1 |

In a particularly preferred embodiment; under in vitro conditions in 900 mL 0.1 N HCl (pH 1.0), using the paddle method according to Ph. Eur. at 50 rpm, after 1 h under physiological conditions, the monolithic pharmaceutical dosage form has released at most 55%, more preferably at most 50%, still more preferably at most 45%, most preferably at most 42% and in particular at most 39% of the first pharmacologically active ingredient (A₁) relative to the total amount of the first pharmacologically active ingredient (A₁) originally contained in the pharmaceutical dosage form.

In another particularly preferred embodiment; under in vitro conditions in 900 mL 0.1 N HCl (pH 1.0), using the paddle method according to Ph. Eur. at 50 rpm, after 30 min under physiological conditions, the monolithic pharmaceutical dosage form has released at most 50%, more preferably at most 45%, still more preferably at most 40%, even more preferably at most 35%, yet more preferably at most 30%, most preferably at most 28% and in particular at most 26% of the first pharmacologically active ingredient (A₁) relative to the total amount of the first pharmacologically active ingredient (A₁) originally contained in the pharmaceutical dosage form.

In another preferred embodiment, the first segment (S₁) contains a first pharmacologically active ingredient (A₁) and a further pharmacologically active ingredient (A_{f}). According to this embodiment, preferably, the first pharmacologically active ingredient (A₁), the further pharmacologically active ingredient (A_{f}) and either the immediate release matrix material or the prolonged release matrix material are intimately homogeneously distributed within the first segment(s) (S₁) so that the first segment(s) (S₁) do(es) not contain any portions where either the first pharmacologically active ingredient (A₁) is present in the absence of the further pharmacologically active ingredient (A_{f}) and either the immediate release matrix material or the prolonged release matrix material; or where the further pharmacologically active ingredient (A_{f}) is present in the absence of the first pharmacologically active ingredient (A₁) and either the immediate release matrix material or the prolonged release matrix material; or where either the immediate release matrix material or the prolonged release matrix material is present in the absence of the first pharmacologically active ingredient (A₁) and the further pharmacologically active ingredient (A_{f}).

The further pharmacologically active ingredient (A_{f}) is preferably different from the first pharmacologically active ingredient (A₁).

Any preferred embodiment which has been defined above with respect to the chemical nature of the first pharmacologically active ingredient (A₁) also applies to the further pharmacologically active ingredient (A_{f}) and is therefore not repeated hereinafter.

Preferably, when the first segment (S₁) comprises a first pharmacologically active ingredient (A₁) and a further pharmacologically active ingredient (A_{f}), the further pharmacologically active ingredient (A_{f}) is present in the monolithic pharmaceutical dosage form in a therapeutically effective amount. In general, the amount that constitutes a therapeutically effective amount varies according to the pharmacologically active ingredients being used, the condition being treated, the severity of said condition, the patient being treated, and whether the monolithic pharmaceutical dosage form or the segment in which the pharmacologically active ingredient is contained is designed for an immediate or retarded release.

With regard to the content of the further pharmacologically active ingredient (A_{f}) relative to the total weight of the first segment(s) (S₁) and relative to the total weight of the monolithic pharmaceutical dosage form, respectively, any preferred embodiment which has been defined above with respect to the content of the first pharmacologically active ingredient (A₁) also applies accordingly to the content of the further pharmacologically active ingredient (A_{f}) and is therefore not repeated hereinafter.

In a preferred embodiment, the relative weight ratio of the total content of the further pharmacologically active ingredient (A_{f}) to the total content of the first pharmacologically active ingredient (A₁) [A_{f}:A₁] is within the range of (60±10):1, (50±10):1, (40±10):1, (30±10):1, (20±5):1, (15±5):1, (10±5):1, (5±3):1, (3±2):1, (2±1):1 or 1:1.

In another preferred embodiment, the relative weight ratio of the total content of the first pharmacologically active ingredient (A₁) to the total content of the further pharmacologically active ingredient (A_{f}) [A₁:A_{f}] is within the range of (60±10):1, (50±10):1, (40±10):1, (30±10):1, (20±5):1, (15±5):1, (10±5):1, (5±3):1, (3±2):1, (2±1):1 or 1:1.

When the first segment (S₁) comprises a first pharmacologically active ingredient (A₁) and a further pharmacologically active ingredient (A_{f}), the first segment (S₁) preferably releases the first pharmacologically active ingredient (A₁) and the further pharmacologically active ingredient (A_{f}) according to the same release mode. In this regard, the term “release mode” preferably only refers to the general terms “prolonged release” or “immediate release”, i.e. two compounds which are released according to the same release mode still can vary in the respective absolute amounts which are released during a given time interval.

Accordingly, when the first pharmacologically active ingredient (A₁) and the further pharmacologically active ingredient (A_{f}) are released from segment (S₁) according to the same release mode, they preferably both display either an immediate release profile or a prolonged release profile.

In a preferred embodiment, the segment (S₁) provides immediate release of the first pharmacologically active ingredient (A₁) and the further pharmacologically active ingredient (A_{f}). According to this embodiment, any preferred embodiment which has been defined above with respect to the immediate release of the first pharmacologically active ingredient (A₁) also applies to the immediate release of the further pharmacologically active ingredient (A_{f}) and is therefore not repeated hereinafter.

In another preferred embodiment, the segment (S₁) provides prolonged release of the first pharmacologically active ingredient (A₁) and the further pharmacologically active ingredient (A_{f}). According to this embodiment, any preferred embodiment which has been defined above with respect to the prolonged release of the first pharmacologically active ingredient (A₁) also applies to the prolonged release of the further pharmacologically active ingredient (A_{f}) and is therefore not repeated hereinafter.

Preferably, the release profile, the preferably present first pharmacologically active ingredient (A₁), the optionally present further pharmacologically active ingredient (A_{f}) and optionally present pharmaceutical excipients of the first segment (S₁) are stable upon storage, preferably upon storage at elevated temperature, e.g. 40° C., for 3 months in sealed containers.

In connection with the release profile “stable” preferably means that when comparing the initial release profile with the release profile after storage, at any given time point the release profiles deviate from one another by not more than 20%, more preferably not more than 15%, still more preferably not more than 10%, yet more preferably not more than 7.5%, most preferably not more than 5.0% and in particular not more than 2.5%.

In connection with a pharmacologically active ingredient and pharmaceutical excipients “stable” preferably means that the segments and the monolithic pharmaceutical dosage form satisfy the requirements of EMEA concerning shelf-life of pharmaceutical products.

Preferably, after storage for 4 weeks, more preferably 6 months, at 40° C. and 75% rel. humidity, the content of the preferably present first pharmacologically active ingredient (A₁) in the first segment(s) (S₁) and the pharmaceutical dosage form, respectively, amounts to at least 98.0%, more preferably at least 98.5%, still more preferably at least 99.0%, yet more preferably at least 99.2%, most preferably at least 99.4% and in particular at least 99.6%, of its original content before storage.

In a preferred embodiment, the first segment(s) (S₁) has/have a breaking strength of at least 300 N.

Preferably, the mechanical properties, particularly the breaking strength, substantially relies on the presence and spatial distribution of the prolonged release matrix material, although its mere presence does typically not suffice in order to achieve said properties.

In another preferred embodiment, the mechanical properties, particularly the breaking strength, substantially relies on the presence and spatial distribution of the immediate release matrix material, although its mere presence does typically not suffice in order to achieve said properties.

The advantageous mechanical properties may not automatically be achieved by simply processing pharmacologically active ingredient, the optional prolonged release matrix material or the optional immediate release matrix material, and optionally further excipients by means of conventional processes for the preparation of pharmaceutical dosage forms. In fact, usually suitable apparatuses must be selected for the preparation and critical processing parameters must be adjusted, particularly pressure/force, temperature and time. Thus, even if conventional apparatuses are used, the process protocols usually must be adapted in order to meet the required criteria.

In general, the desired properties may be obtained only if, during preparation of the first segment(s) (S₁),
suitable components
in suitable amounts are exposed to
a sufficient pressure
at a sufficient temperature
for a sufficient period of time.

Thus, regardless of the apparatus used, the process protocols must be adapted in order to meet the required criteria. Therefore, the breaking strength is separable from the composition.

The first segment(s) (S₁) preferably has/have a breaking strength of at least 300 N, at least 400 N, or at least 500 N, preferably at least 600 N, more preferably at least 700 N, still more preferably at least 800 N, yet more preferably at least 1000 N, most preferably at least 1250 N and in particular at least 1500 N.

The “breaking strength” (resistance to crushing) of a pharmaceutical dosage form or a segment is known to the skilled person. In this regard it can be referred to, e.g., W. A. Ritschel, Die Tablette, 2. Auflage, Editio Cantor Verlag Aulendorf, 2002; H Liebermann et al., Pharmaceutical dosage forms: Pharmaceutical dosage forms, Vol. 2, Informa Healthcare; 2 edition, 1990; and Encyclopedia of Pharmaceutical Technology, Informa Healthcare; 1 edition.

For the purpose of the specification, the breaking strength is preferably defined as the amount of force that is necessary in order to fracture a pharmaceutical dosage form and a segment, respectively (=breaking force). Therefore, for the purpose of the specification a monolithic pharmaceutical dosage form and segment, respectively, does preferably not exhibit the desired breaking strength when it breaks, i.e., is fractured into at least two independent parts that are separated from one another. In another preferred embodiment, however, the monolithic pharmaceutical dosage form and segment, respectively, is regarded as being broken if the force decreases by 25% (threshold value) of the highest force measured during the measurement (see below).

Preferably, the first segment (S₁) according to the invention is distinguished from conventional pharmaceutical dosage forms and segments, respectively, in that due to its breaking strength, it cannot be pulverized by the application of force with conventional means, such as for example a pestle and mortar, a hammer, a mallet or other usual means for pulverization, in particular devices developed for this purpose (pharmaceutical dosage form crushers). In this regard “pulverization” means crumbling into small particles. Avoidance of pulverization virtually rules out oral or parenteral, in particular intravenous or nasal abuse.

Preferably, the first segment (S₁) is tamper resistant and provides resistance against grinding.

Conventional pharmaceutical dosage forms and segments, respectively, typically have a breaking strength well below 200 N.

The breaking strength of conventional round pharmaceutical dosage forms and segments, respectively, may be estimated according to the following empirical formula:

Breaking Strength[in N]=10×Diameter of pharmaceutical dosage form/segment[in mm].

Thus, according to said empirical formula, a round pharmaceutical dosage form/segment having a breaking strength of at least 300 N would require a diameter of at least 30 mm. Such a particle however, could not be swallowed, let alone a pharmaceutical dosage form containing such a particle. The above empirical formula preferably does not apply to the first segment (S₁) according to the invention, which is not conventional but rather special.

Further, the actual mean chewing force is about 220 N (cf., e.g., P. A. Proeschel et al., J Dent Res, 2002, 81(7), 464-468). This means that conventional pharmaceutical dosage forms and segments, respectively, having a breaking strength well below 200 N may be crushed upon spontaneous chewing, whereas the first segment (S₁) according to the invention may preferably not.

Still further, when applying a gravitational acceleration of about 9.81 m/s², 300 N correspond to a gravitational force of more than 30 kg, i.e. the first segment (S₁) according to the invention can preferably withstand a weight of more than 30 kg without being pulverized.

Methods for measuring the breaking strength are known to the skilled artisan. Suitable devices are commercially available.

For example, the breaking strength (resistance to crushing) can be measured in accordance with the Eur. Ph. 5.0, 2.9.8 or 6.0, 2.09.08 “Resistance to Crushing of Pharmaceutical dosage forms”. The segments may be subjected to the same or similar breaking strength test as the pharmaceutical dosage form. The test is intended to determine, under defined conditions, the resistance to crushing of pharmaceutical dosage forms and segments, respectively, measured by the force needed to disrupt them by crushing. The apparatus consists of 2 jaws facing each other, one of which moves towards the other. The flat surfaces of the jaws are perpendicular to the direction of movement. The crushing surfaces of the jaws are flat and larger than the zone of contact with the pharmaceutical dosage form and segments, respectively. The apparatus is calibrated using a system with a precision of 1 Newton. The pharmaceutical dosage form and segment, respectively, is placed between the jaws, taking into account, where applicable, the shape, the break-mark and the inscription; for each measurement the pharmaceutical dosage form and segment, respectively, is oriented in the same way with respect to the direction of application of the force (and the direction of extension in which the breaking strength is to be measured). The measurement is carried out on 10 pharmaceutical dosage forms and segments, respectively, taking care that all fragments have been removed before each determination. The result is expressed as the mean, minimum and maximum values of the forces measured, all expressed in Newton.

A similar description of the breaking strength (breaking force) can be found in the USP. The breaking strength can alternatively be measured in accordance with the method described therein where it is stated that the breaking strength is the force required to cause a pharmaceutical dosage form and segment(s), respectively, to fail (i.e., break) in a specific plane. The pharmaceutical dosage form and segment(s), respectively, is generally placed between two platens, one of which moves to apply sufficient force to the pharmaceutical dosage form and segment, respectively, to cause fracture. For conventional, round (circular cross-section) pharmaceutical dosage form and segments, respectively, loading occurs across their diameter (sometimes referred to as diametral loading), and fracture occurs in the plane. The breaking force of pharmaceutical dosage form and segment, respectively, is commonly called hardness in the pharmaceutical literature; however, the use of this term is misleading. In material science, the term hardness refers to the resistance of a surface to penetration or indentation by a small probe. The term crushing strength is also frequently used to describe the resistance of pharmaceutical dosage form and segments, respectively, to the application of a compressive load. Although this term describes the true nature of the test more accurately than does hardness, it implies that pharmaceutical dosage form and segments, respectively, are actually crushed during the test, which is often not the case.

Alternatively, the breaking strength (resistance to crushing) can be measured in accordance with WO 2008/107149, which can be regarded as a modification of the method described in the Eur. Ph. The apparatus used for the measurement is preferably a “Zwick Z 2.5” materials tester, Fₘₐₓ=2.5 kN with a maximum draw of 1150 mm, which should be set up with one column and one spindle, a clearance behind of 100 mm and a test speed adjustable between 0.1 and 800 mm/min together with testControl software. Measurement is performed using a pressure piston with screw-in inserts and a cylinder (diameter 10 mm), a force transducer, Fₘₐₓ. 1 kN, diameter=8 mm, class 0.5 from 10 N, class 1 from 2 N to ISO 7500-1, with manufacturer's test certificate M according to DIN 55350-18 (Zwick gross force Fₘₐₓ=1.45 kN) (all apparatus from Zwick GmbH & Co. KG, Ulm, Germany) with Order No BTC-FR 2.5 TH. D09 for the tester, Order No BTC-LC 0050N. P01 for the force transducer, Order No BO 70000 S06 for the centring device.

In a preferred embodiment, the monolithic pharmaceutical dosage form and segment, respectively, is regarded as being broken if it is fractured into at least two separate pieces.

When the first segment (S₁) and the second segment (S₂) have a different breaking strength, the breaking strengths of both segments may be determined separately or together, depending upon the geometry of the dosage form and the relative position of the segments. For example, when the segment with the lower breaking strength surrounds the segment with the higher breaking strength, (cf. FIG. 1) the dosage form may be as such subjected to a conventional breaking strength test. As a result, the weaker outer segment will break first thus providing a first breaking strength value and the stronger inner segment will break subsequently providing a second breaking strength value. However, it is also possible to separate the segments from one another and to measure their breaking strength separately and independently. Separation of the segments may be achieved e.g. by means of a knife having a metal blade that may be heated, or by any other means available to a skilled person. Alternatively, the segments may be prepared separately of one another and the breaking strength of the separated segments may be measured independently.

The first segment(s) (S₁) according to the invention preferably exhibit(s) mechanical strength over a wide temperature range, in addition to the breaking strength (resistance to crushing) optionally also sufficient hardness, impact resistance, impact elasticity, tensile strength and/or modulus of elasticity, optionally also at low temperatures (e.g. below −24° C., below −40° C. or possibly even in liquid nitrogen), for it to be virtually impossible to pulverize by spontaneous chewing, grinding in a mortar, pounding, etc. Thus, preferably, the comparatively high breaking strength of the first segment(s) (S₁) according to the invention is maintained even at low or very low temperatures, e.g., when the monolithic pharmaceutical dosage form is initially chilled to increase its brittleness, for example to temperatures below −25° C., below −40° C. or even in liquid nitrogen.

The first segment(s) (S₁) according to the invention is/are characterized by a certain degree of breaking strength. This does not mean that it must also exhibit a certain degree of hardness. Hardness and breaking strength are different physical properties. Therefore, the preferred tamper-resistance of the first segment(s) (S₁) does not necessarily depend on the hardness of the first segment(s) (S₁). For instance, due to its breaking strength, impact strength, elasticity modulus and tensile strength, respectively, the first segment(s) (S₁) can preferably be deformed, e.g. plastically, when exerting an external force, for example using a hammer, but cannot be pulverized, i.e., crumbled into a high number of fragments. In other words, the first segment(s) (S₁) according to the invention is/are characterized by a certain degree of breaking strength, but not necessarily also by a certain degree of form stability.

Therefore, in the meaning of the specification, a pharmaceutical dosage form and segment, respectively, that is deformed when being exposed to a force in a particular direction of extension but that does not break (plastic deformation or plastic flow) is preferably to be regarded as having the desired breaking strength in said direction of extension.

Preferred pharmaceutical dosage forms and segments, respectively, are those having a suitable tensile strength as determined by a test method currently accepted in the art. Further pharmaceutical dosage form and segments, respectively, are those having a Youngs Modulus as determined by a test method of the art. Still further pharmaceutical dosage form and segments, respectively, are those having an acceptable elongation at break.

In a preferred embodiment, the segment (S₁) is tamper-resistant and/or exhibits a breaking strength of at least 300 N. In another preferred embodiment, the segment (S₁) is tamper-resistant and exhibits a breaking strength of at least 300 N.

In a further preferred embodiment, the segment (S₁) which is tamper-resistant and exhibits a breaking strength of at least 300 N provides resistance against grinding and/or resistance against solvent extraction and/or resistance against dose-dumping in aqueous ethanol.

Tamper-resistant preferably means that the first segment(s) (S₁)
(i) preferably provide(s) resistance against solvent extraction, and/or
(ii) preferably provide(s) resistance against grinding, and/or
(iii) preferably provide(s) resistance against dose-dumping in aqueous ethanol.

Thus, the first segment(s) (S₁) according to the invention do(es) not necessarily need to exhibit any of resistances (i) to (iii); but may preferably exhibit any of resistances (i) to (iii) as well as any combination thereof; namely only (i); only (ii); only (iii); a combination of only (i) and (ii); a combination of only (i) and (iii); a combination of only (ii) and (iii); or a combination of (i) and (ii) and (iii).

Preferably, when the first segment(s) (S₁) provide(s) prolonged release of the preferably present first pharmacologically active ingredient (A₁), the prolonged release of A₁ is achieved by a prolonged release matrix contained in the first segment(s) (S₁) wherein said prolonged release matrix additionally provides tamper resistance in terms of resistance against solvent extraction, resistance against grinding, and resistance against dose-dumping in aqueous ethanol.

As used herein, the term “tamper-resistant” refers to pharmaceutical dosage forms or segments that are resistant to conversion into a form suitable for misuse or abuse, particular for nasal and/or intravenous administration, by conventional means.

In this regard, the monolithic pharmaceutical dosage form as such it may be crushable by conventional means such as grinding in a mortar or crushing by means of a hammer. However, the first segment(s) (S₁) contained in the monolithic pharmaceutical dosage form preferably exhibit(s) mechanical properties such that they cannot be pulverized by conventional means any further. As the first segment(s) (S₁) is/are of macroscopic size and contain(s) the pharmacologically active ingredient, it/they cannot be administered nasally thereby rendering the monolithic pharmaceutical dosage form tamper-resistant.

Preferably, the prolonged release matrix of the first segment(s) (S₁) provides resistance against solvent extraction.

Preferably, when trying to tamper the monolithic pharmaceutical dosage form in order to prepare a formulation suitable for abuse by intravenous administration, the liquid part of the formulation that can be separated from the remainder by means of a syringe at room temperature is as less as possible, preferably it contains not more than 45 or 40 wt.-%, more preferably not more than 35 wt.-%, still more preferably not more than 30 wt.-%, yet more preferably not more than 25 wt.-%, even more preferably not more than 20 wt.-%, most preferably not more than 15 wt.-% and in particular not more than 10 wt.-% of the preferably originally contained first pharmacologically active ingredient (A₁).

Preferably, this property is tested by (i) dispensing a monolithic pharmaceutical dosage form that is either intact or has been manually comminuted by means of two spoons in 5 ml of solvent, either purified water or aqueous ethanol (40 vol. %), (ii) allowing the dispersion to stand for 10 min at room temperature, (iii) drawing up the hot liquid into a syringe (needle 21G equipped with a cigarette filter), and (iv) determining the amount of the pharmacologically active ingredient contained in the liquid within the syringe.

Preferably, when the first segment(s) (S₁) comprise(s) a prolonged release matrix, said prolonged release matrix provides resistance against.

Preferably, when the first segment(s) (S₁) is/are treated with a commercial coffee mill, preferably type Bosch MKM6000, 180W, Typ KM13 for 2 minutes, 42±17.5 wt.-%, more preferably 42±15 wt.-%, still more preferably 42±12.5 wt.-%, yet more preferably 42±10 wt.-%, even more preferably 42±7.5 wt.-%, most preferably 42±5 wt.-%, and in particular 42±2.5 wt.-%, of the total weight of the thus obtained material does not pass a sieve having a mesh size of 1.000 mm.

Preferably, when the first segment(s) (S₁) is/are treated with a commercial coffee mill, preferably type Bosch MKM6000, 180W, Typ KM13, for 2 minutes, 57±17.5 wt.-%, more preferably 57±15 wt.-%, still more preferably 57±12.5 wt.-%, yet more preferably 57±10 wt.-%, even more preferably 57±7.5 wt.-%, most preferably 57±5 wt.-%, and in particular 57±2.5 wt.-%, of the total weight of the thus obtained material does not pass a sieve having a mesh size of 1.000 mm.

Preferably, when the first segment(s) (S₁) is/are treated with a commercial coffee mill, preferably type Bosch MKM6000, 180W, Typ KM13, for 2 minutes, at least 50 wt.-%, more preferably at least 55 wt.-%, still more preferably at least 60 wt.-%, yet more preferably at least 65 wt.-%, even more preferably at least 70 wt.-%, most preferably at least 75 wt.-%, and in particular at least 80 wt.-%, of the total weight of the thus obtained material does not pass a sieve having a mesh size of 1.000 mm.

Preferably, when the monolithic pharmaceutical dosage form is treated with a commercial coffee mill, preferably type Bosch MKM6000, 180W, Typ KM13 for 2 minutes, 42±17.5 wt.-%, more preferably 42±15 wt.-%, still more preferably 42±12.5 wt.-%, yet more preferably 42±10 wt.-%, even more preferably 42±7.5 wt.-%, most preferably 42±5 wt.-%, and in particular 42±2.5 wt.-%, of the total weight of the thus obtained material does not pass a sieve having a mesh size of 1.000 mm.

Preferably, when the monolithic pharmaceutical dosage form is/are treated with a commercial coffee mill, preferably type Bosch MKM6000, 180W, Typ KM13, for 2 minutes, 57±17.5 wt.-%, more preferably 57±15 wt.-%, still more preferably 57±12.5 wt.-%, yet more preferably 57±10 wt.-%, even more preferably 57±7.5 wt.-%, most preferably 57±5 wt.-%, and in particular 57±2.5 wt.-%, of the total weight of the thus obtained material does not pass a sieve having a mesh size of 1.000 mm.

Preferably, when the monolithic pharmaceutical dosage form is treated with a commercial coffee mill, preferably type Bosch MKM6000, 180W, Typ KM13, for 2 minutes, at least 50 wt.-%, more preferably at least 55 wt.-%, still more preferably at least 60 wt.-%, yet more preferably at least 65 wt.-%, even more preferably at least 70 wt.-%, most preferably at least 75 wt.-%, and in particular at least 80 wt.-%, of the total weight of the thus obtained material does not pass a sieve having a mesh size of 1.000 mm.

Particle size distributions of the ground pharmaceutical dosage form are preferably determined by sieve analysis.

In a preferred embodiment, after treatment with a commercial coffee mill as described above, more than 55%, more preferably more than 60%, still more preferably more than 65%, yet more preferably more than 70%, most preferably 75% and in particular more than 80% of the particles of the ground first segment (S₁) and the ground monolithic pharmaceutical dosage form, respectively, have a size in the range of from 0.2 to 3.3 nm, more preferably of from 0.4 to 3.1 nm, most preferably of from 0.6 to 2.9 and in particular of from 0.7 to 2.8 nm.

Preferred particle size distributions P¹ to P⁶ are summarized in the table underneath:

| particle size | amount [wt.-%%] | | | | | |
|---|---|---|---|---|---|---|
| [nm] | P¹ | P² | P³ | P⁴ | P⁵ | P⁶ |
| <0.045 | 0.5 ± 0.4 | 0.1 ± 0.09 | 0.3 ± 0.29 | 0.3 ± 0.29 | 0.3 ± 0.29 | 0.3 ± 0.29 |
| 0.045-0.063 | 0.5 ± 0.4 | 0.3 ± 0.29 | 0.3 ± 0.29 | 0.3 ± 0.29 | 0.3 ± 0.29 | 0.3 ± 0.29 |
| 0.063-0.090 | 0.5 ± 0.4 | 0.3 ± 0.29 | 0.3 ± 0.29 | 1.0 ± 0.9 | 0.3 ± 0.29 | 0.3 ± 0.29 |
| 0.090-0.125 | 0.5 ± 0.4 | 0.3 ± 0.29 | 0.3 ± 0.29 | 1.0 ± 0.9 | 0.3 ± 0.29 | 1.0 ± 0.9 |
| 0.125-0.180 | 0.5 ± 0.4 | 3.0 ± 2.9 | 2.0 ± 1.5 | 2.0 ± 1.5 | 1.0 ± 0.9 | 1.0 ± 0.9 |
| 0.180-0.250 | 1.5 ± 1.4 | 1.0 ± 0.8 | 2.0 ± 1.5 | 1.0 ± 0.9 | 2.0 ± 1.5 | 1.0 ± 0.9 |
| 0.250-0.355 | 4.0 ± 3.5 | 5.0 ± 4.0 | 4.0 ± 3.5 | 3.5 ± 2.5 | 5.0 ± 4.0 | 3.0 ± 2.9 |
| 0.355-0.500 | 7.0 ± 6.0 | 5.0 ± 4.0 | 6.0 ± 4.5 | 7.0 ± 6.0 | 7.0 ± 6.0 | 7.0 ± 6.0 |
| 0.500-0.710 | 11.0 ± 8.0 | 9.0 ± 7.0 | 11.0 ± 8.0 | 10.0 ± 7.0 | 13.0 ± 10.0 | 9.0 ± 7.0 |
| 0.710-1.000 | 15.0 ± 12.0 | 10.0 ± 7.0 | 17.0 ± 14.0 | 18.0 ± 15.0 | 18.0 ± 15.0 | 13.0 ± 10.0 |
| 1.000-1.400 | 20.0 ± 17.0 | 18.0 ± 15.0 | 23.0 ± 20.0 | 28.0 ± 25.0 | 25.0 ± 22.0 | 20.0 ± 17.0 |
| 1.400-2.000 | 23.0 ± 20.0 | 19.0 ± 16.0 | 12.0 ± 9.0 | 18.0 ± 15.0 | 10.0 ± 7.0 | 22.0 ± 19.0 |
| 2.000-2.800 | 13.0 ± 10.0 | 16.0 ± 13.0 | 13.0 ± 10.0 | 11.0 ± 8.0 | 14.0 ± 11.0 | 12.0 ± 9.0 |
| 2.800-4.000 | 1.0 ± 0.8 | 14.0 ± 11.0 | 12.0 ± 9.0 | 0.3 ± 0.29 | 4.0 ± 3.5 | 9.0 ± 7.0 |
| >4.00 | 0.5 ± 0.45 | 0.3 ± 0.29 | 0.3 ± 0.29 | 0.5 ± 0.45 | 0.3 ± 0.29 | 0.5 ± 0.45 |

Preferably, when the first segment(s) (S₁) comprise a prolonged release matrix, said prolonged release matrix provides resistance against dose-dumping in aqueous ethanol.

The monolithic pharmaceutical dosage form can be tested in vitro using ethanol/simulated gastric fluid of 0%, 20% and 40% to evaluate alcohol extractability. Testing is preferably performed using standard procedures, e.g. USP Apparatus 1 (basket) or USP Apparatus 2 (paddle) at e.g. 50 rpm in e.g. 500 ml of media at 37° C., using a Perkin Elmer UV/VIS Spectrometer Lambda 20, UV at an appropriate wavelength for detection of the first pharmacologically active ingredient (A₁) present therein. Sample time points preferably include 0.5 and 1 hour.

Preferably, when comparing the in vitro release profile at 37° C. in simulated gastric fluid with the in vitro release profile in ethanol/simulated gastric fluid (40 vol.-%) at 37° C., the in vitro release in ethanol/simulated gastric fluid (40 vol.-%) is preferably not substantially accelerated compared to the in vitro release in simulated gastric fluid. Preferably, in this regard “substantially” means that at any given time point the in vitro release in ethanol/simulated gastric fluid (40 vol.-%) relatively deviates from the in vitro release in simulated gastric fluid by not more than +25%, more preferably not more than +20%, still more preferably not more than +15%, yet more preferably not more than +10%, even more preferably not more than +7.5%, most preferably not more than +5.0% and in particular not more than +2.5%.

A substantial relative acceleration of the in vitro release in ethanol/simulated gastric fluid (40 vol.-%) compared to the in vitro release in simulated gastric fluid is to be prevented according to the invention. However, a substantial relative deceleration of the in vitro release in ethanol/simulated gastric fluid (40 vol.-%) compared to the in vitro release in simulated gastric fluid, e.g., a relative deviation by −25% or more, may be possible and can even be desirable.

The second segment(s) (S₂) preferably comprise(s) the second pharmacologically active ingredient (A₂).

In another preferred embodiment, the second segment (S₂) does not contain any pharmacologically active ingredient.

In a preferred embodiment, the segment (S₂) provides immediate release of the second pharmacologically active ingredient (A₂). In another preferred embodiment, the segment (S₂) provides prolonged release of the second pharmacologically active ingredient (A₂).

In a particularly preferred embodiment, the second segment (S₂) is hot melt extruded and comprises the second pharmacologically active ingredient (A₂).

The second pharmacologically active ingredient (A₂) may be identical to or different from the first pharmacologically active ingredient (A₁).

In a preferred embodiment, the second pharmacologically active ingredient (A₂) is different from the first pharmacologically active ingredient (A₁). In another preferred embodiment, the second pharmacologically active ingredient (A₂) is identical to the first pharmacologically active ingredient (A₁).

Any preferred embodiment which has been defined above with respect to the chemical nature of the first pharmacologically active ingredient (A₁) also applies to the second pharmacologically active ingredient (A₂) and is therefore not repeated hereinafter.

In a preferred embodiment, the second pharmacologically active ingredient (A₂) exhibits no psychotropic action.

In another preferred embodiment, the second pharmacologically active ingredient (A₂) is selected from ATC classes [M01A], [M01d], [N02B] and [N02C] according to the WHO.

In still another preferred embodiment, the second pharmacologically active ingredient (A₂) is paracetamol (acetaminophen) or ibuprofen, more preferably paracetamol.

The second pharmacologically active ingredient (A₂) is preferably present in the monolithic pharmaceutical dosage form in a therapeutically effective amount. In general, the amount that constitutes a therapeutically effective amount varies according to the pharmacologically active ingredients being used, the condition being treated, the severity of said condition, the patient being treated, and whether the monolithic pharmaceutical dosage form or the segment in which the pharmacologically active ingredient is contained is designed for an immediate or retarded release.

With regard to the content of the preferably present second pharmacologically active ingredient (A₂) relative to the total weight of the second segment(s) (S₂) and relative to the total weight of the monolithic pharmaceutical dosage form, respectively, any preferred embodiment which has been defined above with respect to the content of the first pharmacologically active ingredient (A₁) also applies accordingly to the content of the second pharmacologically active ingredient (A₂) and is therefore not repeated hereinafter.

In a preferred embodiment, the relative weight ratio of the total content of the second pharmacologically active ingredient (A₂) to the total content of the first pharmacologically active ingredient (A₁) [A₂:A₁] is within the range of (60±10):1, (50±10):1, (40±10):1, (30±10):1, (20±5):1, (15±5):1, (10±5):1, (5±3):1, (3±2):1, (2±1):1 or 1:1.

In another preferred embodiment, the relative weight ratio of the total content of the first pharmacologically active ingredient (A₁) to the total content of the second pharmacologically active ingredient (A₂) [A₁:A₂] is within the range of (60±10):1, (50±10):1, (40±10):1, (30±10):1, (20±5):1, (15±5):1, (10±5):1, (5±3):1, (3±2):1, (2±1):1 or 1:1.

In a preferred embodiment, the segment (S₂) provides immediate release of the second pharmacologically active ingredient (A₂).

Any preferred embodiment which has been defined above with respect to the immediate release of the first pharmacologically active ingredient (A₁) also applies to the immediate release of the second pharmacologically active ingredient (A₂) and is therefore not repeated hereinafter.

In another preferred embodiment, the segment (S₂) provides prolonged release of the second pharmacologically active ingredient (A₂).

Any preferred embodiment which has been defined above with respect to the prolonged release of the first pharmacologically active ingredient (A₁) also applies to the prolonged release of the second pharmacologically active ingredient (A₂) and is therefore not repeated hereinafter.

In another preferred embodiment, the second segment (S₂) contains a second pharmacologically active ingredient (A₂) and a further pharmacologically active ingredient (A_{f}). According to this embodiment, preferably, the second pharmacologically active ingredient (A₂), the further pharmacologically active ingredient (A_{f}) and either the immediate release matrix material or the prolonged release matrix material are intimately homogeneously distributed within the first segment(s) (S₂) so that the first segment(s) (S₂) do(es) not contain any portions where either the second pharmacologically active ingredient (A₂) is present in the absence of the further pharmacologically active ingredient (A_{f}) and either the immediate release matrix material or the prolonged release matrix material; or where the further pharmacologically active ingredient (A_{f}) is present in the absence of the second pharmacologically active ingredient (A₂) and either the immediate release matrix material or the prolonged release matrix material; or where either the immediate release matrix material or the prolonged release matrix material is present in the absence of the second pharmacologically active ingredient (A₂) and the further pharmacologically active ingredient (A_{f}).

The further pharmacologically active ingredient (A_{f}) is preferably different from the second pharmacologically active ingredient (A₂).

Any preferred embodiment which has been defined above with respect to the chemical nature of the first pharmacologically active ingredient (A₁) also applies to the further pharmacologically active ingredient (A_{f}) and is therefore not repeated hereinafter.

Preferably, when the second segment (S₂) comprises a second pharmacologically active ingredient (A₂) and a further pharmacologically active ingredient (A_{f}), the further pharmacologically active ingredient (A_{f}) is present in the monolithic pharmaceutical dosage form in a therapeutically effective amount. In general, the amount that constitutes a therapeutically effective amount varies according to the pharmacologically active ingredients being used, the condition being treated, the severity of said condition, the patient being treated, and whether the monolithic pharmaceutical dosage form or the segment in which the pharmacologically active ingredient is contained is designed for an immediate or retarded release.

With regard to the content of the further pharmacologically active ingredient (A_{f}) relative to the total weight of the second segment(s) (S₂) and relative to the total weight of the monolithic pharmaceutical dosage form, respectively, any preferred embodiment which has been defined above with respect to the content of the first pharmacologically active ingredient (A₁) also applies accordingly to the content of the further pharmacologically active ingredient (A_{f}) and is therefore not repeated hereinafter.

In a preferred embodiment, the relative weight ratio of the total content of the further pharmacologically active ingredient (A_{f}) to the total content of the second pharmacologically active ingredient (A₂) [A_{f}:A₂] is within the range of (60±10):1, (50±10):1, (40±10):1, (30±10):1, (20±5):1, (15±5):1, (10±5):1, (5±3):1, (3±2):1, (2±1):1 or 1:1.

In another preferred embodiment, the relative weight ratio of the total content of the second pharmacologically active ingredient (A₂) to the total content of the further pharmacologically active ingredient (A_{f}) [A₂:A_{f}] is within the range of (60±10): 1, (50±10): 1, (40±10): 1, (30±10):1, (20±5):1, (15±5):1, (10±5):1, (5±3):1, (3±2):1, (2±1):1 or 1:1.

When the second segment (S₂) comprises a second pharmacologically active ingredient (A₂) and a further pharmacologically active ingredient (A_{f}), the second segment (S₂) preferably releases the second pharmacologically active ingredient (A₂) and the further pharmacologically active ingredient (A_{f}) according to the same release mode. In this regard, the term “release mode” preferably has the meaning which has already been defined hereinabove.

In a preferred embodiment, the segment (S₂) provides immediate release of the second pharmacologically active ingredient (A₂) and the further pharmacologically active ingredient (A_{f}). According to this embodiment, any preferred embodiment which has been defined above with respect to the immediate release of the first pharmacologically active ingredient (A₁) also applies to the immediate release of the second pharmacologically active ingredient (A₂) and the further pharmacologically active ingredient (A_{f}) and is therefore not repeated hereinafter.

In another preferred embodiment, the segment (S₂) provides prolonged release of the second pharmacologically active ingredient (A₂) and the further pharmacologically active ingredient (A_{f}). According to this embodiment, any preferred embodiment which has been defined above with respect to the prolonged release of the first pharmacologically active ingredient (A₁) also applies to the prolonged release of the second pharmacologically active ingredient (A₂) and the further pharmacologically active ingredient (A_{f}) and is therefore not repeated hereinafter.

Preferably, the release profile, the preferably present second pharmacologically active ingredient (A₂), the optionally present further pharmacologically active ingredient (A_{f}) and optionally present pharmaceutical excipients of the second segment (S₂) are stable upon storage, preferably upon storage at elevated temperature, e.g. 40° C., for 3 months in sealed containers.

Preferably, after storage for 4 weeks, more preferably 6 months, at 40° C. and 75% rel. humidity, the content of the preferably present second pharmacologically active ingredient (A₂) in the second segment(s) (S₂) and the pharmaceutical dosage form, respectively, amounts to at least 98.0%, more preferably at least 98.5%, still more preferably at least 99.0%, yet more preferably at least 99.2%, most preferably at least 99.4% and in particular at least 99.6%, of its original content before storage.

In a preferred embodiment, the second segment(s) (S₂) has/have a breaking strength of less than 300 N.

In another preferred embodiment, the second segment(s) (S₂) has/have a breaking strength of at most 500 N, more preferably at most 300 N, still more preferably at most 250 N, yet more preferably at most 200 N, even more preferably at most 150 N, most preferably at most 100 N, and in particular at most 50 N.

In still another preferred embodiment, the second segment(s) (S₂) has/have a breaking strength of at least 300 N.

The second segment(s) (S₂) preferably has/have a breaking strength of at least 300 N, at least 400 N, or at least 500 N, preferably at least 600 N, more preferably at least 700 N, still more preferably at least 800 N, yet more preferably at least 1000 N, most preferably at least 1250 N and in particular at least 1500 N.

Preferably, the mechanical properties, particularly the breaking strength, substantially relies on the presence and spatial distribution of the prolonged release matrix material, although its mere presence does typically not suffice in order to achieve said properties.

In another preferred embodiment, the mechanical properties, particularly the breaking strength, substantially relies on the presence and spatial distribution of the immediate release matrix material, although its mere presence does typically not suffice in order to achieve said properties.

In still another preferred embodiment, the mechanical properties, particularly the breaking strength, substantially relies on the presence of an armoring layer. According to this embodiment, the second segment (S₂) preferably is in form of an armoring layer.

Preferred compounds which can be contained in the armoring layer are selected from the group comprised of polyvinyl caprolactames, anionic acrylic polymers and cationic acrylic polymers.

Preferred polyvinyl caprolactames which may be contained in the armoring layer include polyvinyl caprolactam-polyvinyl acetate-polyethylene glycol graft copolymers which are also commercially available as Soluplus®.

Preferred anionic acrylic polymers which may be contained in the armoring layer include copolymers of one or two different C₁₋₄-alkyl(meth)acrylate monomers and copolymerizable anionic monomers such as acrylic acid. Preferred anionic acrylic polymers are commercially available as Eudragit® FS 100.

Preferred cationic acrylic polymers which may be contained in the armoring layer include cationic copolymers based on dimethylaminoethyl methacrylate, butyl methacrylate, and methyl methacrylate. Preferred cationic acrylic polymers are commercially available as Eudragit® E 100.

Preferably, the second segment (S₂) according to the invention is distinguished from conventional pharmaceutical dosage forms and segments, respectively, in that due to its breaking strength, it cannot be pulverized by the application of force with conventional means, such as for example a pestle and mortar, a hammer, a mallet or other usual means for pulverization, in particular devices developed for this purpose (pharmaceutical dosage form crushers). In this regard “pulverization” means crumbling into small particles. Avoidance of pulverization virtually rules out oral or parenteral, in particular intravenous or nasal abuse.

Preferably, the second segment (S₂) is tamper resistant and provides resistance against grinding.

In a preferred embodiment, the segment (S₂) is tamper-resistant and/or exhibits a breaking strength of at least 300 N. In another preferred embodiment, the segment (S₂) is tamper-resistant and exhibits a breaking strength of at least 300 N.

In a further preferred embodiment, the segment (S₂) which is tamper-resistant and exhibits a breaking strength of at least 300 N provides resistance against grinding and/or resistance against solvent extraction and/or resistance against dose-dumping in aqueous ethanol.

In another preferred embodiment, the segment (S₁) and/or the segment (S₂) which is tamper-resistant and exhibits a breaking strength of at least 300 N provides resistance against grinding and/or resistance against solvent extraction and/or resistance against dose-dumping in aqueous ethanol.

Any preferred embodiment which has been defined above with respect to the tamper resistance of the segment (S₁) also applies to the tamper resistance of the segment (S₂) and is therefore not repeated hereinafter.

In a preferred embodiment, the segment (S₁) exhibits a higher breaking strength than the segment (S₂). In another preferred embodiment, the segment (S₁) is tamper-resistant and exhibits a breaking strength of at least 300 N and the segment (S₂) exhibits a lower breaking strength than the first segment (S₁).

Preferably, the breaking strength of the first segment(s) (S₁) is relatively at least 50 N higher, more preferably at least 100 N higher, still more preferably at least 150 N higher, yet more preferably at least 200 N higher, even more preferably at least 250 N higher, most preferably at least 300 N higher, and in particular at least 350 N higher than the breaking strength of the second segment(s) (S₂).

In a preferred embodiment,
the first segment (S₁) exhibits a breaking strength of at least 300 N, more preferably at least 400 N, still more preferably more than 500 N, yet more preferably at least 750 N, even more preferably at least 1000 N, most preferably at least 1250 N, and in particular at least 1500 N; and/or
the second segment (S₂) exhibits a breaking strength of at most 500 N, more preferably at most 300 N, still more preferably at most 250 N, yet more preferably at most 200 N, even more preferably at most 150 N, most preferably at most 100 N, and in particular at most 50 N.

In another preferred embodiment, the segment (S₂) exhibits a higher breaking strength than the segment (S₁). In still another preferred embodiment, the segment (S₂) is tamper-resistant and exhibits a breaking strength of at least 300 N and the segment (S₁) exhibits a lower breaking strength than the segment (S₂).

Preferably, the breaking strength of the second segment(s) (S₂) is relatively at least 50 N higher, more preferably at least 100 N higher, still more preferably at least 150 N higher, yet more preferably at least 200 N higher, even more preferably at least 250 N higher, most preferably at least 300 N higher, and in particular at least 350 N higher than the breaking strength of the first segment(s) (S₁).

In another preferred embodiment,
the first segment (S₁) exhibits a breaking strength of at most 500 N, more preferably at most 300 N, still more preferably at most 250 N, yet more preferably at most 200 N, even more preferably at most 150 N, most preferably at most 100 N, and in particular at most 50 N; and/or
the second segment (S₂) exhibits a breaking strength of at least 300 N, more preferably at least 400 N, still more preferably more than 500 N, yet more preferably at least 750 N, even more preferably at least 1000 N, most preferably at least 1250 N, and in particular at least 1500 N.

In a further preferred embodiment, the segment (S₁) and the segment (S₂) are each tamper-resistant and each exhibit a breaking strength of at least 300 N, more preferably at least 400 N, still more preferably more than 500 N, yet more preferably at least 750 N, even more preferably at least 1000 N, most preferably at least 1250 N, and in particular at least 1500 N.

In a preferred embodiment, both, the segment (S₁) and the segment (S₂) are hot melt extruded. According to this embodiment, both, the segment (S₁) and the segment (S₂) preferably are tamper-resistant and/or exhibit a breaking strength of at least 300 N.

In another preferred embodiment, the segment (S₁) is hot melt extruded and the segment (S₂) is not hot melt extruded. According to this embodiment, both, the segment (S₁) and the segment (S₂) preferably are tamper-resistant and/or exhibit a breaking strength of at least 300 N.

The segment (S₁) and/or the segment (S₂) preferably provides prolonged release of the pharmacologically active ingredient (A₁) or (A₂) contained therein.

In a preferred embodiment, the first segment (S₁) provides prolonged release of the first pharmacologically active ingredient (A₁) and the second segment (S₂) provides immediate release of the second pharmacologically active ingredient (A₂).

In another preferred embodiment, the first segment (S₁) provides prolonged release of the first pharmacologically active ingredient (A₁) and the second segment (S₂) provides prolonged release of the second pharmacologically active ingredient (A₂).

When the first segment (S₁) and the second segment (S₂) provide prolonged release of the first pharmacologically active ingredient (A₁) and prolonged release of the second pharmacologically active ingredient (A₂), the prolonged release profiles of A₁ and A₂ preferably differ from each other, e.g. in their release rate or in their onset of release.

In still another preferred embodiment, the first segment (S₁) provides immediate release of the first pharmacologically active ingredient (A₁) and the second segment (S₂) provides prolonged release of the second pharmacologically active ingredient (A₂).

In a further preferred embodiment, the first segment (S₁) provides prolonged release of both, the first pharmacologically active ingredient (A₁) and a further pharmacologically active ingredient (A_{f}), whereas the second segment (S₂) does not contain any pharmacologically active ingredient. In still a further preferred embodiment, the first segment (S₁) provides immediate release of both, the first pharmacologically active ingredient (A₁) and a further pharmacologically active ingredient (A_{f}), whereas the second segment (S₂) does not contain any pharmacologically active ingredient.

In yet a further preferred embodiment, the second segment (S₂) provides prolonged release of both, the second pharmacologically active ingredient (A₂) and a further pharmacologically active ingredient (A_{f}), whereas the first segment (S₁) does not contain any pharmacologically active ingredient. In another preferred embodiment, the second segment (S₂) provides immediate release of both, the second pharmacologically active ingredient (A₂) and a further pharmacologically active ingredient (A_{f}), whereas the first segment (S₁) does not contain any pharmacologically active ingredient.

In still another preferred embodiment, the first segment (S₁) provides prolonged release of the first pharmacologically active ingredient (A₁), whereas the second segment (S₂) does not contain any pharmacologically active ingredient. In yet another preferred embodiment, the first segment (S₁) provides immediate release of the first pharmacologically active ingredient (A₁), whereas the second segment (S₂) does not contain any pharmacologically active ingredient.

In another preferred embodiment, the second segment (S₂) provides prolonged release of the second pharmacologically active ingredient (A₂), whereas the first segment (S₁) does not contain any pharmacologically active ingredient. In still another preferred embodiment, the second segment (S₂) provides immediate release of the second pharmacologically active ingredient (A₂), whereas the first segment (S₁) does not contain any pharmacologically active ingredient.

In a preferred embodiment, the first pharmacologically active ingredient (A₁) has a psychotropic effect and the second pharmacologically active ingredient (A₂) is selected from ATC classes [M01A], [M01d], [N02B] and [N02C] according to the WHO.

In another preferred embodiment, the first pharmacologically active ingredient (A₁) is selected from ATC classes [M01A], [M01d], [N02B] and [N02C] according to the WHO and the second pharmacologically active ingredient (A₂) has a psychotropic effect.

Preferred combinations C¹ to C⁵⁶ of the first pharmacologically active ingredient (A₁) and the second pharmacologically active ingredient (A₂) are summarized in the table here below, wherein the first pharmacologically active ingredient (A₁) as well as the second pharmacologically active ingredient (A₂) each also refer to the physiologically acceptable salts thereof, particularly to the hydrochlorides:

| | A₁ | A₂ |
|---|---|---|
| C¹ | oxycodone | ibuprofen |
| C² | oxymorphone | ibuprofen |
| C³ | hydrocodone | ibuprofen |
| C⁴ | hydromorphone | ibuprofen |
| C⁵ | morphine | ibuprofen |
| C⁶ | tapentadol | ibuprofen |
| C⁷ | tramadol | ibuprofen |
| C⁸ | oxycodone | paracetamol |
| C⁹ | oxymorphone | paracetamol |
| C¹⁰ | hydrocodone | paracetamol |
| C¹¹ | hydromorphone | paracetamol |
| C¹² | morphine | paracetamol |
| C¹³ | tapentadol | paracetamol |
| C¹⁴ | tramadol | paracetamol |
| C¹⁵ | oxycodone | diclofenac |
| C¹⁶ | oxymorphone | diclofenac |
| C¹⁷ | hydrocodone | diclofenac |
| C¹⁸ | hydromorphone | diclofenac |
| C¹⁹ | morphine | diclofenac |
| C²⁰ | tapentadol | diclofenac |
| C²¹ | tramadol | diclofenac |
| C²² | oxycodone | acetylsalicylic acid |
| C²³ | oxymorphone | acetylsalicylic acid |
| C²⁴ | hydrocodone | acetylsalicylic acid |
| C²⁵ | hydromorphone | acetylsalicylic acid |
| C²⁶ | morphine | acetylsalicylic acid |
| C²⁷ | tapentadol | acetylsalicylic acid |
| C²⁸ | tramadol | acetylsalicylic acid |
| C²⁹ | ibuprofen | oxycodone |
| C³⁰ | ibuprofen | oxymorphone |
| C³¹ | ibuprofen | hydrocodone |
| C³² | ibuprofen | hydromorphone |
| C³³ | ibuprofen | morphine |
| C³⁴ | ibuprofen | tapentadol |
| C³⁵ | ibuprofen | tramadol |
| C³⁶ | paracetamol | oxycodone |
| C³⁷ | paracetamol | oxymorphone |
| C³⁸ | paracetamol | hydrocodone |
| C³⁹ | paracetamol | hydromorphone |
| C⁴⁰ | paracetamol | morphine |
| C⁴¹ | paracetamol | tapentadol |
| C⁴² | paracetamol | tramadol |
| C⁴³ | diclofenac | oxycodone |
| C⁴⁴ | diclofenac | oxymorphone |
| C⁴⁵ | diclofenac | hydrocodone |
| C⁴⁶ | diclofenac | hydromorphone |
| C⁴⁷ | diclofenac | morphine |
| C⁴⁸ | diclofenac | tapentadol |
| C⁴⁹ | diclofenac | tramadol |
| C⁵⁰ | acetylsalicylic acid | oxycodone |
| C⁵¹ | acetylsalicylic acid | oxymorphone |
| C⁵² | acetylsalicylic acid | hydrocodone |
| C⁵³ | acetylsalicylic acid | hydromorphone |
| C⁵⁴ | acetylsalicylic acid | morphine |
| C⁵⁵ | acetylsalicylic acid | tapentadol |
| C⁵⁶ | acetylsalicylic acid | tramadol |

In another preferred embodiment, the first pharmacologically active ingredient (A₁) has a psychotropic effect and the second pharmacologically active ingredient (A₂) has a psychotropic effect, wherein the first pharmacologically active ingredient (A₁) is identical to or different from the second pharmacologically active ingredient (A₂).

Further preferred combinations C⁵⁷ to C¹⁰⁵ of the first pharmacologically active ingredient (A₁) and the second pharmacologically active ingredient (A₂) are summarized in the table here below, wherein the first pharmacologically active ingredient (A₁) as well as the second pharmacologically active ingredient (A₂) each also refer to the physiologically acceptable salts thereof, particularly to the hydrochlorides:

| | A₁ | A₂ |
|---|---|---|
| C⁵⁷ | oxycodone | oxycodone |
| C⁵⁸ | oxymorphone | oxycodone |
| C⁵⁹ | hydrocodone | oxycodone |
| C⁶⁰ | hydromorphone | oxycodone |
| C⁶¹ | morphine | oxycodone |
| C⁶² | tapentadol | oxycodone |
| C⁶³ | tramadol | oxycodone |
| C⁶⁴ | oxycodone | oxymorphone |
| C⁶⁵ | oxymorphone | oxymorphone |
| C⁶⁶ | hydrocodone | oxymorphone |
| C⁶⁷ | hydromorphone | oxymorphone |
| C⁶⁸ | morphine | oxymorphone |
| C⁶⁹ | tapentadol | oxymorphone |
| C⁷⁰ | tramadol | oxymorphone |
| C⁷¹ | oxycodone | hydrocodone |
| C⁷² | oxymorphone | hydrocodone |
| C⁷³ | hydrocodone | hydrocodone |
| C⁷⁴ | hydromorphone | hydrocodone |
| C⁷⁵ | morphine | hydrocodone |
| C⁷⁶ | tapentadol | hydrocodone |
| C⁷⁷ | tramadol | hydrocodone |
| C⁷⁸ | oxycodone | hydromorphone |
| C⁷⁹ | oxymorphone | hydromorphone |
| C⁸⁰ | hydrocodone | hydromorphone |
| C⁸¹ | hydromorphone | hydromorphone |
| C⁸² | morphine | hydromorphone |
| C⁸³ | tapentadol | hydromorphone |
| C⁸⁴ | tramadol | hydromorphone |
| C⁸⁵ | oxycodone | morphine |
| C⁸⁶ | oxymorphone | morphine |
| C⁸⁷ | hydrocodone | morphine |
| C⁸⁸ | hydromorphone | morphine |
| C⁸⁹ | morphine | morphine |
| C⁹⁰ | tapentadol | morphine |
| C⁹¹ | tramadol | morphine |
| C⁹² | oxycodone | tapentadol |
| C⁹³ | oxymorphone | tapentadol |
| C⁹⁴ | hydrocodone | tapentadol |
| C⁹⁵ | hydromorphone | tapentadol |
| C⁹⁶ | morphine | tapentadol |
| C⁹⁷ | tapentadol | tapentadol |
| C⁹⁸ | tramadol | tapentadol |
| C⁹⁹ | oxycodone | tramadol |
| C¹⁰⁰ | oxymorphone | tramadol |
| C¹⁰¹ | hydrocodone | tramadol |
| C¹⁰² | hydromorphone | tramadol |
| C¹⁰³ | morphine | tramadol |
| C¹⁰⁴ | tapentadol | tramadol |
| C¹⁰⁵ | tramadol | tramadol |

Preferably, when the first pharmacological ingredient (A₁) and the second pharmacologically active ingredient (A₂) are identical to each other, e.g. according to the preferred combinations C⁵⁷, C⁶⁵, C⁷³, C⁸¹, C⁸⁹, C⁹⁷ and C¹⁰⁵, the release profile of the first segment (S₁) containing the first pharmacological ingredient (A₁) is different from the release profile of the second segment (S₂) containing the second pharmacologically active ingredient (A₂).

In a preferred embodiment, when the first pharmacological ingredient (A₁) and the second pharmacologically active ingredient (A₂) are identical to each other, the first segment (S₁) provides prolonged release of the first pharmacological ingredient (A₁) and the second segment (S₂) provides immediate release of the second pharmacologically active ingredient (A₂).

In another preferred embodiment, when the first pharmacological ingredient (A₁) and the second pharmacologically active ingredient (A₂) are identical to each other, the first segment (S₁) provides immediate release of the first pharmacological ingredient (A₁) and the second segment (S₂) provides prolonged release of the second pharmacologically active ingredient (A₂).

In a particularly preferred embodiment,
the first segment (S₁) provides prolonged release of the first pharmacologically active ingredient (A₁), wherein the first pharmacologically active ingredient (A₁) has a psychotropic effect; and
the second segment (S₂) provides immediate release or prolonged release of the second pharmacologically active ingredient (A₂), wherein the second pharmacologically active ingredient (A₂)
   is selected from ATC classes [M01A], [M01C], [N02B] and [N02C] according to the WHO; or
   has a psychotropic effect, wherein the first pharmacologically active ingredient (A₁) is identical to or different from the second pharmacologically active ingredient (A₂).

In another particularly preferred embodiment,
the first segment (S₁) provides immediate release or prolonged release of the first pharmacologically active ingredient (A₁), wherein the first pharmacologically active ingredient (A₁) is selected from ATC classes [M01A], [M01d], [N02B] and [N02C] according to the WHO; and
the second segment (S₂) provides prolonged release of the second pharmacologically active ingredient (A₂), wherein the second pharmacologically active ingredient (A₂) has a psychotropic effect.

Preferred combinations X¹ to X⁶⁶ are summarized in the table here below:

| | | | | | | | position^{c} | | | breaking strength | |
|---|---|---|---|---|---|---|---|---|---|---|---|
| | API^{a} | | | release^{b} | | | of | manufacture | | [N] | |
| | A₁ | A₂ | A_{f} | A₁ | A₂ | A_{f} | A_{f} | S₁ | S₂ | S₁ | S₂ |
| X¹ | + | + | − | PR | PR | − | − | hot melt | hot melt | ≧300 | ≧300 |
| | | | | | | | | extruded | extruded | | |
| X² | + | + | − | PR | PR | − | − | hot melt | hot melt | <300 | ≧300 |
| | | | | | | | | extruded | extruded | | |
| X³ | + | + | − | PR | PR | − | − | hot melt | hot melt | ≧300 | <300 |
| | | | | | | | | extruded | extruded | | |
| X⁴ | + | + | − | IR | PR | − | − | hot melt | hot melt | ≧300 | ≧300 |
| | | | | | | | | extruded | extruded | | |
| X⁵ | + | + | − | IR | PR | − | − | hot melt | hot melt | <300 | ≧300 |
| | | | | | | | | extruded | extruded | | |
| X⁶ | + | + | − | IR | PR | − | − | hot melt | hot melt | ≧300 | <300 |
| | | | | | | | | extruded | extruded | | |
| X⁷ | + | + | − | PR | IR | − | − | hot melt | hot melt | ≧300 | ≧300 |
| | | | | | | | | extruded | extruded | | |
| X⁸ | + | + | − | PR | IR | − | − | hot melt | hot melt | <300 | ≧300 |
| | | | | | | | | extruded | extruded | | |
| X⁹ | + | + | − | PR | IR | − | − | hot melt | hot melt | ≧300 | <300 |
| | | | | | | | | extruded | extruded | | |
| X¹⁰ | + | + | − | PR | PR | − | − | hot melt | not hot | ≧300 | ≧300 |
| | | | | | | | | extruded | melt | | |
| | | | | | | | | | extruded | | |
| X¹¹ | + | + | − | PR | PR | − | − | hot melt | not hot | <300 | ≧300 |
| | | | | | | | | extruded | melt | | |
| | | | | | | | | | extruded | | |
| X¹² | + | + | − | PR | PR | − | − | hot melt | not hot | ≧300 | <300 |
| | | | | | | | | extruded | melt | | |
| | | | | | | | | | extruded | | |
| X¹³ | + | + | − | IR | PR | − | − | hot melt | not hot | ≧300 | ≧300 |
| | | | | | | | | extruded | melt | | |
| | | | | | | | | | extruded | | |
| X¹⁴ | + | + | − | IR | PR | − | − | hot melt | not hot | <300 | ≧300 |
| | | | | | | | | extruded | melt | | |
| | | | | | | | | | extruded | | |
| X¹⁵ | + | + | − | IR | PR | − | − | hot melt | not hot | ≧300 | <300 |
| | | | | | | | | extruded | melt | | |
| | | | | | | | | | extruded | | |
| X¹⁶ | + | + | − | PR | IR | − | − | hot melt | not hot | ≧300 | ≧300 |
| | | | | | | | | extruded | melt | | |
| | | | | | | | | | extruded | | |
| X¹⁷ | + | + | − | PR | IR | − | − | hot melt | not hot | <300 | ≧300 |
| | | | | | | | | extruded | melt | | |
| | | | | | | | | | extruded | | |
| X¹⁸ | + | + | − | PR | IR | − | − | hot melt | not hot | ≧300 | <300 |
| | | | | | | | | extruded | melt | | |
| | | | | | | | | | extruded | | |
| X¹⁹ | + | − | + | PR | − | PR | S₁ | hot melt | hot melt | ≧300 | ≧300 |
| | | | | | | | | extruded | extruded | | |
| X²⁰ | + | − | + | PR | − | PR | S₁ | hot melt | hot melt | <300 | ≧300 |
| | | | | | | | | extruded | extruded | | |
| X²¹ | + | − | + | PR | − | PR | S₁ | hot melt | hot melt | ≧300 | <300 |
| | | | | | | | | extruded | extruded | | |
| X²² | + | − | + | IR | − | IR | S₁ | hot melt | hot melt | ≧300 | ≧300 |
| | | | | | | | | extruded | extruded | | |
| X²³ | + | − | + | IR | − | IR | S₁ | hot melt | hot melt | <300 | ≧300 |
| | | | | | | | | extruded | extruded | | |
| X²⁴ | + | − | + | IR | − | IR | S₁ | hot melt | hot melt | ≧300 | <300 |
| | | | | | | | | extruded | extruded | | |
| X²⁵ | + | − | + | PR | − | PR | S₁ | hot melt | not hot | ≧300 | ≧300 |
| | | | | | | | | extruded | melt | | |
| | | | | | | | | | extruded | | |
| X²⁶ | + | − | + | PR | − | PR | S₁ | hot melt | not hot | <300 | ≧300 |
| | | | | | | | | extruded | melt | | |
| | | | | | | | | | extruded | | |
| X²⁷ | + | − | + | PR | − | PR | S₁ | hot melt | not hot | ≧300 | <300 |
| | | | | | | | | extruded | melt | | |
| | | | | | | | | | extruded | | |
| X²⁸ | + | − | + | IR | − | IR | S₁ | hot melt | not hot | ≧300 | ≧300 |
| | | | | | | | | extruded | melt | | |
| | | | | | | | | | extruded | | |
| X²⁹ | + | − | + | IR | − | IR | S₁ | hot melt | not hot | <300 | ≧300 |
| | | | | | | | | extruded | melt | | |
| | | | | | | | | | extruded | | |
| X³⁰ | + | − | + | IR | − | IR | S₁ | hot melt | not hot | ≧300 | <300 |
| | | | | | | | | extruded | melt | | |
| | | | | | | | | | extruded | | |
| X³¹ | − | + | + | PR | − | PR | S₂ | hot melt | hot melt | ≧300 | ≧300 |
| | | | | | | | | extruded | extruded | | |
| X³² | − | + | + | PR | − | PR | S₂ | hot melt | hot melt | <300 | ≧300 |
| | | | | | | | | extruded | extruded | | |
| X³³ | − | + | + | PR | − | PR | S₂ | hot melt | hot melt | ≧300 | <300 |
| | | | | | | | | extruded | extruded | | |
| X³⁴ | − | + | + | IR | − | IR | S₂ | hot melt | hot melt | ≧300 | ≧300 |
| | | | | | | | | extruded | extruded | | |
| X³⁵ | − | + | + | IR | − | IR | S₂ | hot melt | hot melt | <300 | ≧300 |
| | | | | | | | | extruded | extruded | | |
| X³⁶ | − | + | + | IR | − | IR | S₂ | hot melt | hot melt | ≧300 | <300 |
| | | | | | | | | extruded | extruded | | |
| X³⁷ | − | + | + | PR | − | PR | S₂ | hot melt | not hot | ≧300 | ≧300 |
| | | | | | | | | extruded | melt | | |
| | | | | | | | | | extruded | | |
| X³⁸ | − | + | + | PR | − | PR | S₂ | hot melt | not hot | <300 | ≧300 |
| | | | | | | | | extruded | melt | | |
| | | | | | | | | | extruded | | |
| X³⁹ | − | + | + | PR | − | PR | S₂ | hot melt | not hot | ≧300 | <300 |
| | | | | | | | | extruded | melt | | |
| | | | | | | | | | extruded | | |
| X⁴⁰ | − | + | + | IR | − | IR | S₂ | hot melt | not hot | ≧300 | ≧300 |
| | | | | | | | | extruded | melt | | |
| | | | | | | | | | extruded | | |
| X⁴¹ | − | + | + | IR | − | IR | S₂ | hot melt | not hot | <300 | ≧300 |
| | | | | | | | | extruded | melt | | |
| | | | | | | | | | extruded | | |
| X⁴² | − | + | + | IR | − | IR | S₂ | hot melt | not hot | ≧300 | <300 |
| | | | | | | | | extruded | melt | | |
| | | | | | | | | | extruded | | |
| X⁴³ | + | − | − | PR | − | − | − | hot melt | hot melt | ≧300 | ≧300 |
| | | | | | | | | extruded | extruded | | |
| X⁴⁴ | + | − | − | PR | − | − | − | hot melt | hot melt | <300 | ≧300 |
| | | | | | | | | extruded | extruded | | |
| X⁴⁵ | + | − | − | PR | − | − | − | hot melt | hot melt | ≧300 | <300 |
| | | | | | | | | extruded | extruded | | |
| X⁴⁶ | + | − | − | IR | − | − | − | hot melt | hot melt | ≧300 | ≧300 |
| | | | | | | | | extruded | extruded | | |
| X⁴⁷ | + | − | − | IR | − | − | − | hot melt | hot melt | <300 | ≧300 |
| | | | | | | | | extruded | extruded | | |
| X⁴⁸ | + | − | − | IR | − | − | − | hot melt | hot melt | ≧300 | <300 |
| | | | | | | | | extruded | extruded | | |
| X⁴⁹ | + | − | − | PR | − | − | − | hot melt | not hot | ≧300 | ≧300 |
| | | | | | | | | extruded | melt | | |
| | | | | | | | | | extruded | | |
| X⁵⁰ | + | − | − | PR | − | − | − | hot melt | not hot | <300 | ≧300 |
| | | | | | | | | extruded | melt | | |
| | | | | | | | | | extruded | | |
| X⁵¹ | + | − | − | PR | − | − | − | hot melt | not hot | ≧300 | <300 |
| | | | | | | | | extruded | melt | | |
| | | | | | | | | | extruded | | |
| X⁵² | + | − | − | IR | − | − | − | hot melt | not hot | ≧300 | ≧300 |
| | | | | | | | | extruded | melt | | |
| | | | | | | | | | extruded | | |
| X⁵³ | + | − | − | IR | − | − | − | hot melt | not hot | <300 | ≧300 |
| | | | | | | | | extruded | melt | | |
| | | | | | | | | | extruded | | |
| X⁵⁴ | + | − | − | IR | − | − | − | hot melt | not hot | ≧300 | <300 |
| | | | | | | | | extruded | melt | | |
| | | | | | | | | | extruded | | |
| X⁵⁵ | − | + | − | − | PR | − | − | hot melt | hot melt | ≧300 | ≧300 |
| | | | | | | | | extruded | extruded | | |
| X⁵⁶ | − | + | − | − | PR | − | − | hot melt | hot melt | <300 | ≧300 |
| | | | | | | | | extruded | extruded | | |
| X⁵⁷ | − | + | − | − | PR | − | − | hot melt | hot melt | ≧300 | <300 |
| | | | | | | | | extruded | extruded | | |
| X⁵⁸ | − | + | − | − | IR | − | − | hot melt | hot melt | ≧300 | ≧300 |
| | | | | | | | | extruded | extruded | | |
| X⁵⁹ | − | + | − | − | IR | − | − | hot melt | hot melt | <300 | ≧300 |
| | | | | | | | | extruded | extruded | | |
| X⁶⁰ | − | + | − | − | IR | − | − | hot melt | hot melt | ≧300 | <300 |
| | | | | | | | | extruded | extruded | | |
| X⁶¹ | − | + | − | − | PR | − | − | hot melt | not hot | ≧300 | ≧300 |
| | | | | | | | | extruded | melt | | |
| | | | | | | | | | extruded | | |
| X⁶² | − | + | − | − | PR | − | − | hot melt | not hot | <300 | ≧300 |
| | | | | | | | | extruded | melt | | |
| | | | | | | | | | extruded | | |
| X⁶³ | − | + | − | − | PR | − | − | hot melt | not hot | ≧300 | <300 |
| | | | | | | | | extruded | melt | | |
| | | | | | | | | | extruded | | |
| X⁶⁴ | − | + | − | − | IR | − | − | hot melt | not hot | ≧300 | ≧300 |
| | | | | | | | | extruded | melt | | |
| | | | | | | | | | extruded | | |
| X⁶⁵ | − | + | − | − | IR | − | − | hot melt | not hot | <300 | ≧300 |
| | | | | | | | | extruded | melt | | |
| | | | | | | | | | extruded | | |
| X⁶⁶ | − | + | − | − | IR | − | − | hot melt | not hot | ≧300 | <300 |
| | | | | | | | | extruded | melt | | |
| | | | | | | | | | extruded | | |

^{a}“+” indicates that the respective pharmacologically active ingredient is contained in the monolithic pharmaceutical dosage form and “−” indicates that the respective pharmacologically active ingredient is notcontained in the monolithic pharmaceutical dosage form;
^{b}“PR” stands for prolonged release, “IR” stands for immediate release;
^{c}the term “position of A_{f}” refers to the segment in which A_{f} is contained.

Particularly preferred combinations Y¹ to Y²⁰ are summarized in the table here below:

| | | | breaking strength | | | | | |
|---|---|---|---|---|---|---|---|---|
| | | | [N] | | release | | manufacture | |
| | A₁ | A₂ | S₁ | S₂ | A₁ | A₂ | S₁ | S₂ |
| Y¹ | opioid | NSAID | ≧300 | <300 | prolonged | immediate | hot melt | hot melt |
| | | | | | | | extruded | extruded |
| Y² | | | ≧300 | ≧300 | prolonged | immediate | hot melt | hot melt |
| | | | | | | | extruded | extruded |
| Y³ | | | ≧300 | <300 | prolonged | prolonged | hot melt | hot melt |
| | | | | | | | extruded | extruded |
| Y⁴ | | | ≧300 | ≧300 | prolonged | prolonged | hot melt | hot melt |
| | | | | | | | extruded | extruded |
| Y⁵ | opioid | opioid | ≧300 | ≧300 | prolonged | immediate | hot melt | hot melt |
| | | | | | | | extruded | extruded |
| Y⁶ | | | ≧300 | ≧300 | prolonged | prolonged | hot melt | hot melt |
| | | | | | | | extruded | extruded |
| Y⁷ | NSAID | opioid | <300 | ≧300 | immediate | prolonged | hot melt | hot melt |
| | | | | | | | extruded | extruded |
| Y⁸ | | | ≧300 | ≧300 | immediate | prolonged | hot melt | hot melt |
| | | | | | | | extruded | extruded |
| Y⁹ | | | <300 | ≧300 | prolonged | prolonged | hot melt | hot melt |
| | | | | | | | extruded | extruded |
| Y¹⁰ | | | ≧300 | ≧300 | prolonged | prolonged | hot melt | hot melt |
| | | | | | | | extruded | extruded |
| Y¹¹ | opioid | NSAID | ≧300 | <300 | prolonged | immediate | hot melt | not hot |
| | | | | | | | extruded | melt |
| | | | | | | | | extruded |
| Y¹² | | | ≧300 | ≧300 | prolonged | immediate | hot melt | not hot |
| | | | | | | | extruded | melt |
| | | | | | | | | extruded |
| Y¹³ | | | ≧300 | <300 | prolonged | prolonged | hot melt | not hot |
| | | | | | | | extruded | melt |
| | | | | | | | | extruded |
| Y¹⁴ | | | ≧300 | ≧300 | prolonged | prolonged | hot melt | not hot |
| | | | | | | | extruded | melt |
| | | | | | | | | extruded |
| Y¹⁵ | opioid | opioid | ≧300 | ≧300 | prolonged | immediate | hot melt | not hot |
| | | | | | | | extruded | melt |
| | | | | | | | | extruded |
| Y¹⁶ | | | ≧300 | ≧300 | prolonged | prolonged | hot melt | not hot |
| | | | | | | | extruded | melt |
| | | | | | | | | extruded |
| Y¹⁷ | NSAID | opioid | <300 | ≧300 | immediate | prolonged | hot melt | not hot |
| | | | | | | | extruded | melt |
| | | | | | | | | extruded |
| Y¹⁸ | | | ≧300 | ≧300 | immediate | prolonged | hot melt | not hot |
| | | | | | | | extruded | melt |
| | | | | | | | | extruded |
| Y¹⁹ | | | <300 | ≧300 | prolonged | prolonged | hot melt | not hot |
| | | | | | | | extruded | melt |
| | | | | | | | | extruded |
| Y²⁰ | | | ≧300 | ≧300 | prolonged | prolonged | hot melt | not hot |
| | | | | | | | extruded | melt |
| | | | | | | | | extruded |

In a particularly preferred embodiment,
(a) the first segment (S₁) exhibits a breaking strength of at least 300 N and provides prolonged release of the first pharmacologically active ingredient (A₁) contained therein, whereby said first pharmacologically active ingredient (A₁) is an opioid; and
   (a1) the second segment (S₂) exhibits a lower breaking strength than the first segment (S₁) and provides prolonged release of the second pharmacologically active ingredient (A₂) contained therein, whereby said second pharmacologically active ingredient (A₂) is an NSAID; or
   (a2) the second segment (S₂) exhibits a lower breaking strength than the first segment (S₁) and provides immediate release of the second pharmacologically active ingredient (A₂) contained therein, whereby said second pharmacologically active ingredient (A₂) is an NSAID; or
   (a3) the second segment (S₂) exhibits a breaking strength of at least 300 N and provides prolonged release of the second pharmacologically active ingredient (A₂) contained therein, whereby said second pharmacologically active ingredient (A₂) is an NSAID; or
   (a4) the second segment (S₂) exhibits a breaking strength of at least 300 N and provides immediate release of the second pharmacologically active ingredient (A₂) contained therein, whereby said second pharmacologically active ingredient (A₂) is an NSAID; or
   (a5) the second segment (S₂) exhibits a breaking strength of at least 300 N and provides prolonged release of the second pharmacologically active ingredient (A₂) contained therein, whereby said second pharmacologically active ingredient (A₂) is identical to the first pharmacologically active ingredient (A₁); or
   (a6) the second segment (S₂) exhibits a breaking strength of at least 300 N and provides immediate release of the second pharmacologically active ingredient (A₂) contained therein, whereby said second pharmacologically active ingredient (A₂) is identical to the first pharmacologically active ingredient (A₁); or
   (a7) the second segment (S₂) exhibits a breaking strength of at least 300 N and provides prolonged release of the second pharmacologically active ingredient (A₂) contained therein, whereby said second pharmacologically active ingredient (A₂) is an opioid which is different from the first pharmacologically active ingredient (A₁); or
   (a8) the second segment (S₂) exhibits a breaking strength of at least 300 N and provides immediate release of the second pharmacologically active ingredient (A₂) contained therein, whereby said second pharmacologically active ingredient (A₂) is an opioid which is different from the first pharmacologically active ingredient (A₁);
or
(b) the second segment (S₂) exhibits a breaking strength of at least 300 N and provides prolonged release of the second pharmacologically active ingredient (A₂) contained therein, whereby said second pharmacologically active ingredient (A₂) is an opioid; and
   (b1) the first segment (S₁) exhibits a lower breaking strength than the second segment (S₂) and provides prolonged release of the first pharmacologically active ingredient (A₁) contained therein, whereby said first pharmacologically active ingredient (A₁) is an NSAID; or
   (b2) the first segment (S₁) exhibits a lower breaking strength than the second segment (S₂) and provides immediate release of the first pharmacologically active ingredient (A₁) contained therein, whereby said first pharmacologically active ingredient (A₁) is an NSAID; or
   (b3) the first segment (S₁) exhibits a breaking strength of at least 300 N and provides prolonged release of the first pharmacologically active ingredient (A₁) contained therein, whereby said first pharmacologically active ingredient (A₁) is an NSAID; or
   (b4) the first segment (S₁) exhibits a breaking strength of at least 300 N and provides immediate release of the first pharmacologically active ingredient (A₁) contained therein, whereby said first pharmacologically active ingredient (A₁) is an NSAID.

According to the embodiments (a) (i.e. (a1) to (a8)) and (b) (i.e. (b1) to (b4)), preferably the first segment (S₁) as well as the second segment (S₂) are hot melt extruded.

Further particularly preferred combinations Y²¹ to Y³² wherein the first segment (S₁) as well as the second segment (S₂) are hot melt extruded are summarized in the table here below:

| | | breaking | | | |
|---|---|---|---|---|---|
| | | strength | | | |
| | | [N] | | release | |
| | A₁ and A₂ | S₁ | S₂ | A₁ | A₂ |
| Y²¹ | any of C¹ to C²⁸ | ≧300 | <300 | prolonged | immediate |
| Y²² | | ≧300 | ≧300 | prolonged | immediate |
| Y²³ | | ≧300 | <300 | prolonged | prolonged |
| Y²⁴ | | ≧300 | ≧300 | prolonged | prolonged |
| Y²⁵ | any of C⁵⁷, C⁶⁵, C⁷³, | ≧300 | ≧300 | prolonged | immediate |
| Y²⁶ | C⁸¹, C⁸⁹, C⁹⁷ or C¹⁰⁵ | ≧300 | ≧300 | prolonged | prolonged |
| Y²⁷ | any of C⁵⁸ to C⁶⁴, C⁶⁶ | ≧300 | ≧300 | prolonged | immediate |
| Y²⁸ | to C⁷², C⁷⁴ to C⁸⁰, C⁸² | ≧300 | ≧300 | prolonged | prolonged |
| | to C⁸⁸, C⁹⁰ to C⁹⁶ or | | | | |
| | C⁹⁸ to C¹⁰⁴ | | | | |
| Y²⁹ | any of C²⁹ to C⁵⁶ | <300 | ≧300 | immediate | prolonged |
| Y³⁰ | | ≧300 | ≧300 | immediate | prolonged |
| Y³¹ | | <300 | ≧300 | prolonged | prolonged |
| Y³² | | ≧300 | ≧300 | prolonged | prolonged |

In another particularly preferred embodiment, the monolithic pharmaceutical dosage form is a mantle tablet, wherein the relative weight ratio of the first segment (S₁) to the second segment (S₂) is within the range of from 1:1 to 1:3.5; and
(a) the first segment (S₁) exhibits a breaking strength of at least 500 N and provides prolonged release of the first pharmacologically active ingredient (A₁) contained therein, whereby said first pharmacologically active ingredient (A₁) is an opioid; and
   (a1) the second segment (S₂) exhibits a lower breaking strength than the first segment (S₁) and provides prolonged release of the second pharmacologically active ingredient (A₂) contained therein, whereby said second pharmacologically active ingredient (A₂) is an NSAID; or
   (a2) the second segment (S₂) exhibits a lower breaking strength than the first segment (S₁) and provides immediate release of the second pharmacologically active ingredient (A₂) contained therein, whereby said second pharmacologically active ingredient (A₂) is an NSAID; or
   (a3) the second segment (S₂) exhibits a breaking strength of at least 300 N and provides prolonged release of the second pharmacologically active ingredient (A₂) contained therein, whereby said second pharmacologically active ingredient (A₂) is an NSAID.

According to this embodiment (a) (i.e. (a1) to (a3)), preferably the first segment (S₁) as well as the second segment (S₂) are hot melt extruded.

Because the first segment(s) (S₁) and the second segment(s) (S₂) may exhibit different breaking strengths, when measuring the breaking strength of the monolithic pharmaceutical dosage form according to the invention, a distance-to-force diagram can be obtained that contains at least two steps.

In a preferred embodiment, the monolithic pharmaceutical dosage form has an overall breaking strength of at least 300 N, more preferably at least 400 N, still more preferably more than 500 N, yet more preferably at least 750 N, even more preferably at least 1000 N, most preferably at least 1250 N, and in particular at least 1500 N.

Another aspect of the invention relates to a process for the production of a monolithic pharmaceutical dosage form as described above comprising the steps of
(i) hot melt-extruding a first segment (S₁) preferably containing a first pharmacologically active ingredient (A₁); and
(ii) preferably hot melt-extruding a second segment (S₂) preferably containing a second pharmacologically active ingredient (A₂);
   wherein step (i) is performed before, after and/or simultaneously with step (ii).

The first segment(s) (S₁) is/are hot melt-extruded.

Preferably, the first segment(s) (S₁) and the second segment(s) (S₂) are hot melt-extruded.

In a preferred embodiment, hot melt-extrusion is performed by means of a twin-screw-extruder. Melt extrusion preferably provides a melt-extruded strand that is preferably cut into monoliths, which are then optionally compressed and formed. Preferably, compression is achieved by means of a die and a punch from a monolithic mass obtained by melt extrusion. Preferably, the compressing step is preferably carried out with a monolithic mass exhibiting ambient temperature, that is, a temperature in the range from 20 to 25° C.

The strands obtained by way of extrusion can either be subjected to the compression step as such or can be cut prior to the compression step. This cutting can be performed by usual techniques, for example using rotating knives or compressed air, at elevated temperature, e.g. when the extruded stand is still warm due to hot melt extrusion, or at ambient temperature, i.e. after the extruded strand has been allowed to cool down. When the extruded strand is still warm, singulation of the extruded strand into extruded monoliths, is preferably performed by cutting the extruded strand immediately after it has exited the extrusion die.

However, when the extruded strand is cut in the cooled state, subsequent singulation of the extruded strand is preferably performed by optionally transporting the still hot extruded strand by means of conveyor belts, allowing it to cool down and to congeal, and subsequently cutting it. Alternatively, the shaping can take place as described in EP-A 240 906 by the extrudate being passed between two counter-rotating calender rolls and being shaped directly to the first segment (S₁), preferably the segment (S₂) and the monolithic pharmaceutical dosage form, respectively. It is of course also possible to subject the extruded strands to the compression step or to the cutting step when still warm, that is more or less immediately after the extrusion step. The extrusion is preferably carried out by means of a twin-screw extruder.

The segment (S₁) and preferably the segment (S₂) according to the invention may be produced by different hot melt extrusion processes, the particularly preferred of which are explained in greater detail below. Several suitable processes have already been described in the prior art. In this regard it can be referred to, e.g., WO 2005/016313, WO 2005/063214, WO 2005/102286, WO 2006/002883 and WO 2006/082099.

The manufacture of the first segment(s) (S₁) and preferably the second segment(s) (S₂) according to the invention is realized via hot melt extrusion. In this process, the first segment(s) (S₁) and preferably the second segment(s) (S₂) are produced by thermoforming with the assistance of an extruder, preferably without there being any observable consequent discoloration of the extrudate.

This process is preferably characterized in that
a) all components are mixed,
b) the resultant mixture is heated in the extruder at least up to the softening point of preferably the prolonged release matrix material and the immediate release matrix material, respectively, and extruded through the outlet orifice of the extruder by application of force,
c) the still plastic extrudate is singulated and formed into the first segment (S₁) and preferably the second segment (S₂), respectively, or
d) the cooled and optionally reheated singulated extrudate is formed into the first segment (S₁) and preferably the second segment (S₂), respectively.

Mixing of the components according to process step a) may also proceed in the extruder.

The components may also be mixed in a mixer known to the person skilled in the art. The mixer may, for example, be a roll mixer, shaking mixer, shear mixer, compulsory mixer, container mixer or free fall mixer.

The molten mixture which has been heated in the extruder at least up to the softening point of preferably the prolonged release matrix material and the immediate release matrix material, respectively, is extruded from the extruder through a die with at least one bore.

The hot melt extrusion process according to the invention requires the use of suitable extruders, preferably screw extruders. Screw extruders which are equipped with two screws (twin-screw-extruders) are particularly preferred.

In a preferred embodiment, extrusion is performed in the absence of water, i.e., no water is added. However, traces of water (e.g., caused by atmospheric humidity) may be present.

In another preferred embodiment, particularly when a prolonged release matrix material is employed in the form of an aqueous dispersion, extrusion is performed in the presence of water and the water is evaporated from the extruded material in the course of the extrusion process, i.e. preferably before the extruded material exits the outlet orifice of the extruder. Therefore a vacuum pump mechanism is used to extract the (evaporated) water from the extruded material. Thus, the extruded strand is preferably water-free, which preferably means that the water content of the extruded strand is preferably at most 10 wt.-%, or at most 7.5 wt.-%, or at most 5.0 wt.-%, or at most 4.0 wt.-%, or at most 3.0 wt.-%, or at most 2.0 wt.-%, more preferably at most 1.7 wt.-%, still more preferably at most 1.5 wt.-%, yet more preferably at most 1.3 wt.-%, even more preferably at most 1.0 wt.-%, most preferably at most 0.7 wt.-%, and in particular at most 0.5 wt.-%. For that purpose, extrusion is preferably performed at a temperature above the boiling point of water under the given conditions; when extrusion is performed under vacuum, the boiling point of water may be substantially below 100° C. However, even if extrusion is performed under vacuum the preferred extrusion temperature is above 100° C.

The extruder preferably comprises at least two temperature zones, with heating of the mixture at least up to the softening point of preferably the prolonged release matrix material and the immediate release matrix material, respectively, proceeding in the first zone, which is downstream from a feed zone and optionally mixing zone. The throughput of the mixture is preferably from 0.1 kg to 15 kg/hour. In a preferred embodiment, the throughput is from 0.2 kg/hour to 1.7 kg/hour or 3.5 kg/hour. In another preferred embodiment, the throughput is from 4 to 15 kg/hour.

In a preferred embodiment, the die head pressure is within the range of from 0.5 to 200 bar. The die head pressure can be adjusted inter alia by die geometry, temperature profile, extrusion speed, number of bores in the dies, screw configuration, first feeding steps in the extruder, and the like.

In a preferred embodiment, the die head pressure is within the range of from 20±19 bar, more preferably 20±15 bar, and in particular 20±10 bar; or the die head pressure is within the range of from 30±20 bar, more preferably 30±15 bar, and in particular 30±10 bar; or the die head pressure is within the range of from 40±20 bar, more preferably 40±15 bar, and in particular 40±10 bar; or the die head pressure is within the range of from 50±20 bar, more preferably 50±15 bar, and in particular 50±10 bar; or the die head pressure is within the range of from 60±20 bar, more preferably 60±15 bar, and in particular 60±10 bar; or the die head pressure is within the range of from 70±20 bar, more preferably 70±15 bar, and in particular 70±10 bar; or the die head pressure is within the range of from 80±20 bar, more preferably 80±15 bar, and in particular 80±10 bar; or the die head pressure is within the range of from 90±20 bar, more preferably 90±15 bar, and in particular 90±10 bar; or the die head pressure is within the range of from 100±20 bar, more preferably 100±15 bar, and in particular 100±10 bar.

The die geometry or the geometry of the bores is freely selectable. The die or the bores may accordingly exhibit a round, flat (film), oblong or oval cross-section, wherein the round cross-section preferably has a diameter of 0.1 mm to 5 mm. Preferably, the die or the bores have a round cross-section. The casing of the extruder used according to the invention may be heated or cooled. The corresponding temperature control, i.e. heating or cooling, is so arranged that the mixture to be extruded exhibits at least an average temperature (product temperature) corresponding to the softening temperature of preferably the prolonged release matrix material and the immediate release matrix material, respectively, and does not rise above a temperature at which the pharmacologically active ingredient to be processed may be damaged. Preferably, the temperature of the mixture to be extruded is adjusted to below 180° C., preferably below 150° C., but at least to the softening temperature of preferably the prolonged release matrix material and the immediate release matrix material, respectively. Typical extrusion temperatures are 120° C. and 150° C. In a preferred embodiment, the extrusion temperature is in the range of from 95 to 150° C., more preferably 100 to 145° C.

In a preferred embodiment, the extruder torque is within the range of from 30 to 95%. Extruder torque can be adjusted inter alia by die geometry, temperature profile, extrusion speed, number of bores in the dies, screw configuration, first feeding steps in the extruder, and the like.

After extrusion of the molten mixture and optional cooling of the extruded strand or extruded strands, the extrudates are preferably singulated. This singulation may preferably be performed by cutting up the extrudates by means of revolving or rotating knives, wires, blades or with the assistance of laser cutters.

Preferably, intermediate or final storage of the optionally singulated extrudate or the final shape of the first segment (S₁), preferably the second segment (S₂) and the monolithic pharmaceutical dosage form, respectively, is performed under oxygen-free atmosphere which may be achieved, e.g., by means of oxygen-scavengers.

The singulated extrudate may be press-formed in order to impart the final shape to the first segment(s) (S₁), preferably to the second segment (S₂) and to the monolithic pharmaceutical dosage form, respectively.

The application of force in the extruder onto the at least plasticized mixture is adjusted by controlling the rotational speed of the conveying device in the extruder and the geometry thereof and by dimensioning the outlet orifice in such a manner that the pressure necessary for extruding the plasticized mixture is built up in the extruder, preferably immediately prior to extrusion. The extrusion parameters which, for each particular composition, are necessary to give rise to a pharmaceutical dosage form with desired mechanical properties, may be established by simple preliminary testing.

For example but not limiting, extrusion may be performed by means of a twin-screw-extruder type ZSE 18 HP PH 40D or ZSE27 PH 40D Micro (Leistritz, Nürnberg, Germany), screw diameters of 18 or 27 mm or a twin-screw-extruder type Pharma 16 HME (equipped with a vacuum pump, Thermo Fisher Scientific) with a medium shear screw. Screws having eccentric or blunt ends may be used. A heatable die with a single round bore or with a multitude of bores each having a diameter of 0.2, 0.3, 0.4, 0.5, 0.6, 0.7, 0.8, 0.9, 1.0, 2.0, 3.0, 4.0, 5.0 or 6.0 mm may be used. The extrusion parameters may be adjusted e.g. to the following values:
rotational speed of the screws: 70 rpm or 100 rpm; delivery rate 0.5 kg/h for a ZSE27 PH 40D Micro; temperature at the die: 135° C.; or
rotational speed of the screws: 100 rpm, 150 rpm or 200 rpm; delivery rate 0.5 kg/h, 0.8 kg/h, 1.0 kg/h or 1.5 kg/h for a Pharma 16 HME; temperature at the die: 100° C., 105° C., 115° C., 120° C., 130° C., 135° C. or 145° C.; or
rotational speed of the screws: 100 rpm; delivery rate 0.6 kg/h, 0.75 kg/h or 0.8 kg/h for a ZSE 18 HP PH 40D; temperature at the die: 135° C.

Preferably, extrusion is performed by means of twin-screw-extruders or planetary-gear-extruders, twin-screw extruders (co-rotating or contra-rotating) being particularly preferred.

The first segment(s) (S₁) and preferably the second segment(s) (S₂) according to the invention are produced by thermoforming with the assistance of an extruder, preferably without any observable consequent discoloration of the extrudates.

The process for the preparation of the first segment (S₁) and preferably the second segment (S₂), respectively, according to the invention is preferably performed continuously. Preferably, the process involves the extrusion of a homogeneous mixture of all components. It is particularly advantageous if the thus obtained intermediate, e.g. the strand obtained by extrusion, exhibits uniform properties. Particularly desirable are uniform density, uniform distribution of the active compound, uniform mechanical properties, uniform porosity, uniform appearance of the surface, etc. Only under these circumstances the uniformity of the pharmacological properties, such as the stability of the release profile, may be ensured and the amount of rejects can be kept low.

In a preferred embodiment, the first segment (S₁) is monolithic and the monolith according to the invention can be regarded as “extruded pellet”. The term “extruded pellet” has structural implications which are understood by persons skilled in the art. A person skilled in the art knows that a pelletized segment can be prepared by a number of techniques, including:
drug layering on nonpareil sugar or microcrystalline cellulose beads,
spray drying,
spray congealing,
rotogranulation,
hot melt extrusion,
spheronization of low melting materials, or
extrusion-spheronization of a wet mass.

Accordingly, “extruded pellets” can be obtained either by hot melt extrusion or by extrusion-spheronization.

“Extruded pellets” can be distinguished from other types of pellets because they are structurally different. For example, drug layering on nonpareils yields multilayered pellets having a core, whereas extrusion typically yields a monolithic mass comprising a homogeneous mixture of all ingredients. Similarly, spray drying and spray congealing typically yield spheres, whereas extrusion typically yields cylindrical extrudates which can be subsequently spheronized.

The structural differences between “extruded pellets” and “agglomerated pellets” are significant because they may affect the release of active substances from the pellets and consequently result in different pharmacological profiles. Therefore, a person skilled in the pharmaceutical formulation art would not consider “extruded pellets” to be equivalent to “agglomerated pellets”.

The monolithic pharmaceutical dosage forms according to the invention may be prepared from the first segment(s) (S₁) and the second segment(s) (S₂) by any conventional process.

In a particularly preferred embodiment, the process for the production of a monolithic pharmaceutical dosage form as described above comprises the steps of
(i) hot melt-extruding a first segment (S₁) preferably containing a first pharmacologically active ingredient (A₁); and
(ii) preferably hot melt-extruding a second segment (S₂) preferably containing a second pharmacologically active ingredient (A₂);
   wherein step (i) is performed before step (ii).

According to this embodiment, hot melt extrusion of the first segment (S₁) preferably provides an extruded strand having a circular cross section which optionally after being cooled to room temperature and optionally after being cut into strands having a defined length (e.g. approx. 1 m) is sheathed with the second segment (S₂) forming a mantle around the first segment (S₁).

The skilled person knows how to sheath an extruded strand. According to the invention, sheathing of the extruded strand of the first segment (S₁) is preferably realized by introducing said extruded strand in an extruder equipped with a cable sheathing nozzle which allows hot melt-extruding the second segment (S₂) around the surface of the segment (S₁) thereby forming a mantle around the segment (S₁).

Preferably, a cable sheathing nozzle having a circular cross section is employed preferably having an inner diameter of 3 to 5 mm, more preferably about 4 mm, and preferably having an outer diameter of 5.5 to 7 mm, more preferably about 6 mm.

In another preferred embodiment, when hot melt extrusion of the first segment (S₁) is performed before preferably hot melt-extruding the second segment (S₂), the hot melt-extruded segment (S₁) is a flat, sheet-like strand. According to this embodiment, the flat extruded strand of segment (S₁) optionally after being cooled to room temperature and optionally after being cut into strands having a defined length (e.g. approx. 1 m) is provided with the second segment (S₂) forming a flat, sheet-like layer on one of both of the surfaces of the flat extruded strand of segment (S₁).

The skilled person knows how to obtain a flat extruded strand. According to the invention, sheet dies are preferred.

In another particularly preferred embodiment, the process for the production of a monolithic pharmaceutical dosage form as described above comprises the steps of
(i) hot melt-extruding a first segment (S₁) preferably containing a first pharmacologically active ingredient (A₁); and
(ii) preferably hot melt-extruding a second segment (S₂) preferably containing a second pharmacologically active ingredient (A₂);
   wherein step (i) is performed simultaneously with step (ii).

According to this embodiment, the segment (S₁) and the segment (S₂) are preferably obtained by co-extrusion. Co-extrusion and co-extrusion dies are state of the art and well-known to any person skilled in the art.

When co-extruding the first segment (S₁) and the second segment (S₂), the first segment (S₁) preferably has a round cross section and the second segment (S₂) preferably forms a mantle covering the surface of said first segment (S₁); or the first segment(s) (S₁) and the second segment(s) (S₂) are extruded in such a way giving a layered structure.

Preferably, after simultaneous or subsequent hot melt-extrusion of the first segment (S₁) and the second segment (S₂), the resulting strands comprising the first segment (S₁) and the second segment (S₂) are cut into parts containing the desired amount of the first pharmacologically active ingredient (A₁) and the desired amount of the second pharmacologically active ingredient (A₂). Said cut parts are preferably shaped into oblong or round tablets. The skilled person knows how to shape cut extrudates into oblong or round tablets.

When the second segment (S₂) forms a mantle around the first segment (S₁), after shaping the cut parts into an oblong or round tablet form, the second segment (S₂) preferably covers more than 80%, more preferably more than 90%, still more preferably more than 95%, even more preferably more than 99%, most preferably more than 99.9% and in particular more than 99.999% of the surface of the first segment (S₁).

In still another particularly preferred embodiment, the process for the production of a monolithic pharmaceutical dosage form as described above comprises the steps of
(i) hot melt-extruding a first segment (S₁) preferably containing a first pharmacologically active ingredient (A₁); and
(ii) preferably hot melt-extruding a second segment (S₂) preferably containing a second pharmacologically active ingredient (A₂);
   wherein step (i) is performed after step (ii).

According to this embodiment, the preferably hot melt-extruded segment (S₂) is a flat, sheet-like strand which optionally after being cooled to room temperature and optionally after being cut into strands having a defined length (e.g. approx. 1 m) is provided with the first segment (S₁) forming a flat, sheet-like layer on one of the surfaces of the flat preferably extruded strand of segment (S₂).

Another aspect of the invention relates to a monolithic pharmaceutical dosage form that is obtainable by any of the processes described above.

Examples of pharmaceutical dosage forms according to the invention include, but are not limited to tablets, pills, films, effervescent tablets, co-extruded entities and the like.

For the purpose of specification, “co-extruded entities” may refer to any solid pharmaceutical entity which is obtained at least partially by co-extrusion. Extrusion and co-extrusion is state of the art and well-known to any person skilled in the art.

Particularly preferably, the monolithic pharmaceutical dosage form is obtained by co-extrusion.

In a preferred embodiment, the monolithic pharmaceutical dosage form is selected from the group consisting of mantle tablets, layered tablets, mantled layered tablets, co-extruded entities, sugar-coated tablets and dry-coated tablets.

Most pharmaceutical dosage forms are intended to be swallowed whole and accordingly, preferred pharmaceutical dosage forms according to the invention are designed for oral administration. However, alternatively pharmaceutical dosage forms may be dissolved in the mouth, chewed, and some may be placed in a body cavity. Thus, the monolithic pharmaceutical dosage form according to the invention may alternatively be adapted for buccal, lingual, rectal or vaginal administration. Implants are also possible.

The monolithic pharmaceutical dosage form according to the invention has preferably a total weight in the range of 0.01 to 1.5 g, more preferably in the range of 0.05 to 1.2 g, still more preferably in the range of 0.1 g to 1.0 g, yet more preferably in the range of 0.2 g to 0.9 g, and most preferably in the range of 0.2 g to 0.7 g. In a preferred embodiment, the total weight of the monolithic pharmaceutical dosage form is within the range of 250±100 mg, more preferably 250±80 mg, most preferably 250±60 mg, and in particular 250±50 mg. In another preferred embodiment, the total weight of the monolithic pharmaceutical dosage form is within the range of 300±200 mg, more preferably 300±150 mg, most preferably 300±100 mg, and in particular 300±50 mg. In still another preferred embodiment, the total weight of the monolithic pharmaceutical dosage form is within the range of 400±250 mg, more preferably 400±200 mg, still more preferably 400±150 mg, yet more preferably 400±100 mg, most preferably 400±75 mg, and in particular 400±50 mg. In yet another preferred embodiment, the total weight of the monolithic pharmaceutical dosage form is within the range of 500±350 mg, more preferably 500±300 mg, still more preferably 500±200 mg, yet more preferably 500±150 mg, most preferably 500±100 mg, and in particular 500±50 mg.

In a preferred embodiment, the monolithic pharmaceutical dosage form according to the invention is an oblong pharmaceutical dosage form. Pharmaceutical dosage forms of this embodiment preferably have a lengthwise extension (longitudinal extension) of about 1 mm to about 30 mm, in particular in the range of about 2 mm to about 25 mm, more in particular about 5 mm to about 23 mm, even more in particular about 7 mm to about 20 mm; a width in the range of about 1 mm to about 30 mm, in particular in the range of about 2 mm to about 25 mm, more in particular about 5 mm to about 23 mm, even more in particular about 5 mm to about 13 mm; and a thickness in the range of about 1.0 mm to about 12 mm, in particular in the range of about 2.0 mm to about 10 mm, even more in particular from 3.0 mm to about 9.0 mm, even further in particular from about 4.0 mm to about 8.0 mm.

In another preferred embodiment, the monolithic pharmaceutical dosage form according to the invention is a round pharmaceutical dosage form. Pharmaceutical dosage forms of this embodiment preferably have a diameter in the range of about 1 mm to about 30 mm, in particular in the range of about 2 mm to about 25 mm, more in particular about 5 mm to about 23 mm, even more in particular about 7 mm to about 13 mm; and a thickness in the range of about 1.0 mm to about 12 mm, in particular in the range of about 2.0 mm to about 10 mm, even more in particular from 3.0 mm to about 9.0 mm, even further in particular from about 4.0 mm to about 8.0 mm.

Preferably, the monolithic pharmaceutical dosage form according to the invention is not in form of a film.

The monolithic pharmaceutical dosage form according to the invention may optionally comprise a coating, e.g. a cosmetic coating. The coating is preferably applied after formation of the monolithic pharmaceutical dosage form. The pharmaceutical dosage forms according to the invention are preferably film coated with conventional film coating compositions. Suitable coating materials are commercially available, e.g. under the trademarks Opadry® and Eudragit®.

Examples of suitable materials include cellulose esters and cellulose ethers, such as methylcellulose (MC), hydroxypropylmethylcellulose (HPMC), hydroxypropylcellulose (HPC), hydroxyethylcellulose (HEC), sodium carboxymethylcellulose (Na-CMC), poly(meth)-acrylates, such as aminoalkylmethacrylate copolymers, methacrylic acid methylmethacrylate copolymers, methacrylic acid methylmethacrylate copolymers; vinyl polymers, such as polyvinylpyrrolidone, polyvinyl alcohol, polyvinylacetate; and natural film formers.

The coating can be resistant to gastric juices and dissolve as a function of the pH value of the release environment. By means of this coating, it is possible to ensure that the monolithic pharmaceutical dosage form according to the invention passes through the stomach undissolved and the active compound is only released in the intestines. The coating which is resistant to gastric juices preferably dissolves at a pH value of between 5 and 7.5.

The coating can also be applied e.g. to improve the aesthetic impression and/or the taste of the pharmaceutical dosage forms and the ease with which they can be swallowed. Coating the monolithic pharmaceutical dosage forms according to the invention can also serve other purposes, e.g. improving stability and shelf-life. Suitable coating formulations comprise a film forming polymer such as, for example, polyvinyl alcohol or hydroxypropyl methylcellulose, e.g. hypromellose, a plasticizer such as, for example, a glycol, e.g. propylene glycol or polyethylene glycol, an opacifier, such as, for example, titanium dioxide, and a film smoothener, such as, for example, talc. Suitable coating solvents are water as well as organic solvents. Examples of organic solvents are alcohols, e.g. ethanol or isopropanol, ketones, e.g. acetone, or halogenated hydrocarbons, e.g. methylene chloride. Coated monolithic pharmaceutical dosage forms according to the invention are preferably prepared by first making the uncoated monolithic pharmaceutical dosage forms and subsequently coating said uncoated monolithic pharmaceutical dosage forms using conventional techniques, such as coating in a coating pan.

Preferably, the coating does not contain first pharmacologically active ingredient (A₁) and/or second pharmacologically active ingredient (A₂), more preferably the coating does not contain any pharmacologically active ingredient.

Preferably, the coating does not influence the release rate of the first pharmacologically active ingredient (A₁) and/or the second pharmacologically active ingredient (A₂). Further, the coating preferably does not have any openings and is preferably covers more than 99.999% of the total surface of the monolithic pharmaceutical dosage form.

In a preferred embodiment, the monolithic pharmaceutical dosage form according to the invention contains no substances which irritate the nasal passages and/or pharynx, i.e. substances which, when administered via the nasal passages and/or pharynx, bring about a physical reaction which is either so unpleasant for the patient that he/she does not wish to or cannot continue administration, for example burning, or physiologically counteracts taking of the corresponding active compound, for example due to increased nasal secretion or sneezing. Further examples of substances which irritate the nasal passages and/or pharynx are those which cause burning, itching, urge to sneeze, increased formation of secretions or a combination of at least two of these stimuli. Corresponding substances and the quantities thereof which are conventionally to be used are known to the person skilled in the art. Some of the substances which irritate the nasal passages and/or pharynx are accordingly based on one or more constituents or one or more plant parts of a hot substance drug. Corresponding hot substance drugs are known per se to the person skilled in the art and are described, for example, in “Pharmazeutische Biologie—Drogen and ihre Inhaltsstoffe” by Prof. Dr. Hildebert Wagner, 2nd., revised edition, Gustav Fischer Verlag, Stuttgart-New York, 1982, pages 82 et seq. The corresponding description is hereby introduced as a reference and is deemed to be part of the disclosure.

The monolithic pharmaceutical dosage form according to the invention furthermore preferably contains no antagonists for the pharmacologically active ingredients, preferably no antagonists against psychotropic substances, in particular no antagonists against opioids. Antagonists suitable for a given pharmacologically active ingredient are known to the person skilled in the art and may be present as such or in the form of corresponding derivatives, in particular esters or ethers, or in each case in the form of corresponding physiologically acceptable compounds, in particular in the form of the salts or solvates thereof. The monolithic pharmaceutical dosage form according to the invention preferably contains no antagonists selected from among the group comprising naloxone, naltrexone, nalmefene, nalide, nalmexone, nalorphine or naluphine, in each case optionally in the form of a corresponding physiologically acceptable compound, in particular in the form of a base, a salt or solvate; and no neuroleptics, for example a compound selected from among the group comprising haloperidol, promethacine, fluphenazine, perphenazine, levomepromazine, thioridazine, perazine, chlorpromazine, chlorprothixine, zuclopenthixol, flupentixol, prothipendyl, zotepine, benperidol, pipamperone, melperone and bromperidol.

The monolithic pharmaceutical dosage form according to the invention furthermore preferably contains no emetic. Emetics are known to the person skilled in the art and may be present as such or in the form of corresponding derivatives, in particular esters or ethers, or in each case in the form of corresponding physiologically acceptable compounds, in particular in the form of the salts or solvates thereof. The monolithic pharmaceutical dosage form according to the invention preferably contains no emetic based on one or more constituents of ipecacuanha (ipecac) root, for example based on the constituent emetine, as are, for example, described in “Pharmazeutische Biologie—Drogen and ihre Inhaltsstoffe” by Prof. Dr. Hildebert Wagner, 2nd, revised edition, Gustav Fischer Verlag, Stuttgart, New York, 1982. The corresponding literature description is hereby introduced as a reference and is deemed to be part of the disclosure. The monolithic pharmaceutical dosage form according to the invention preferably also contains no apomorphine as an emetic.

Finally, the monolithic pharmaceutical dosage form according to the invention preferably also contains no bitter substance. Bitter substances and the quantities effective for use may be found in US-2003/0064099 A1, the corresponding disclosure of which should be deemed to be the disclosure of the present application and is hereby introduced as a reference. Examples of bitter substances are aromatic oils, such as peppermint oil, eucalyptus oil, bitter almond oil, menthol, fruit aroma substances, aroma substances from lemons, oranges, limes, grapefruit or mixtures thereof, and/or denatonium benzoate.

The monolithic pharmaceutical dosage form according to the invention accordingly preferably contains neither substances which irritate the nasal passages and/or pharynx, nor antagonists for the pharmacologically active ingredients, nor emetics, nor bitter substances.

Preferably, the first segment (S₁) and/or the second segment (S₂), more preferably the entire pharmaceutical dosage form according to the invention contains more than 20 wt.-%, more preferably more than 30 wt.-%, still more preferably more than 40 wt.-%, yet more preferably more than 50 wt.-%, most preferably more than 60 wt.-%, and in particular more than 70 wt.-% of compounds which are not or hardly soluble in ethanol with respect to the total weight of the monolithic pharmaceutical dosage form.

For the purpose of specification, compounds which are not or hardly soluble in ethanol have a maximum solubility in aqueous ethanol (96%) at room temperature of preferably less than 1000 mg/L, more preferably less than 800 mg/L, even more preferably less than 500 mg/L, most preferably less than 100 mg/L and in particular less than 10 mg/L or less than 1 mg/L.

Preferably, the first segment (S₁) and/or the second segment (S₂), more preferably the entire pharmaceutical dosage form according to the invention contains more than 50 wt.-%, more preferably more than 60 wt.-%, still more preferably more than 70 wt.-%, yet more preferably more than 80 wt.-%, most preferably more than 90 wt.-%, and in particular more than 95 wt.-% of polymers which are not or hardly soluble in ethanol with respect to the overall amount of polymers contained in the pharmaceutical dosage form.

Preferred polymers which are not or hardly soluble in ethanol according to the invention are xanthan, guar gum and some types of HPMC. The skilled person knows what types of HPMC are not or hardly soluble in ethanol within the sense of the invention.

In a particularly preferred embodiment, first segment (S₁) and/or the second segment (S₂), more preferably the entire pharmaceutical dosage form according to the invention contains polymers which are not or hardly soluble in ethanol and polymers which are soluble in ethanol, wherein the amount of polymers which are not or hardly soluble in ethanol relative to the total amount of polymers contained in the dosage form is 30 to 100 wt.-%, more preferably 50 to 100 wt.-%, still more preferably 60 to 95 wt.-% or 100 wt.-%, yet more preferably 70 to 90 wt.-% or 100 wt.-%, most preferably 80 to 90 wt.-% or 90 to 100 wt.-%, and in particular more than 95 wt.-% or more than 99 wt.-%.

In a preferred embodiment, the monolithic pharmaceutical dosage form according to the invention is adapted for administration once daily, preferably orally. In another preferred embodiment, the monolithic pharmaceutical dosage form according to the invention is adapted for administration twice daily, preferably orally. In still another preferred embodiment, the monolithic pharmaceutical dosage form according to the invention is adapted for administration thrice daily, preferably orally. In yet another preferred embodiment, the monolithic pharmaceutical dosage form according to the invention is adapted for administration more frequently than thrice daily, for example 4 times daily, 5 times daily, 6 times daily, 7 times daily or 8 times daily, in each case preferably orally.

For the purpose of the specification, “twice daily” means equal or nearly equal time intervals, i.e., about every 12 hours, or different time intervals, e.g., 8 and 16 hours or 10 and 14 hours, between the individual administrations.

For the purpose of the specification, “thrice daily” means equal or nearly equal time intervals, i.e., about every 8 hours, or different time intervals, e.g., 6, 6 and 12 hours; or 7, 7 and 10 hours, between the individual administrations.

The monolithic pharmaceutical dosage forms according to the invention may be used in medicine, e.g. as an analgesic. The monolithic pharmaceutical dosage forms are therefore particularly suitable for the treatment or management of pain. In such pharmaceutical dosage forms, the pharmacologically active ingredients A₁ and A₂ preferably are analgesically effective.

A further aspect of the invention relates to the monolithic pharmaceutical dosage form as described above for use in the treatment of pain.

A further aspect of the invention relates to the use of the first pharmacologically active ingredient (A₁) and of the second pharmacologically active ingredient (A₂) for the manufacture of a monolithic pharmaceutical dosage form as described above for treating pain.

A further aspect of the invention relates to a method of treating pain comprising the administration of the monolithic pharmaceutical dosage form as described above to a subject in need thereof.

A further aspect according to the invention relates to the use of a monolithic pharmaceutical dosage form as described above for avoiding or hindering the abuse of the first pharmacologically active ingredient (A₁) and/or the second pharmacologically active ingredient (A₂) contained therein.

A further aspect according to the invention relates to the use of a monolithic pharmaceutical dosage form as described above for avoiding or hindering the unintentional overdose of the first pharmacologically active ingredient (A₁) and/or second pharmacologically active ingredient (A₂) contained therein.

In this regard, the invention also relates to the use of a monolithic pharmaceutical dosage form as described above for the prophylaxis and/or the treatment of a disorder, thereby preventing an overdose of the first pharmacologically active ingredient (A₁) and/or the second pharmacologically active ingredient (A₂), particularly due to comminution of the monolithic pharmaceutical dosage form by mechanical action.

In a particularly preferred embodiment,
segment (S₁) and segment (S₂) are hot melt extruded; and/or
segment (S₁) contains a first pharmacologically active ingredient (A₁); and/or
segment (S₂) contains a second pharmacologically active ingredient (A₂); and/or
the relative weight ratio of the first segment (S₁) to the second segment (S₂) in the monolithic pharmaceutical dosage form is within the range of from 50:50 to 20:80; and/or
segment (S₁) is tamper-resistant and exhibits a breaking strength of at least 500 N; and/or
segment (S₂) exhibits a lower breaking strength than segment (S₁); and/or
segment (S₂) exhibits a breaking strength of at least 300 N; and/or
segment (S₂) covers at least 99% of the surface of the first segment (S₁); and/or
segment (S₁) provides prolonged release of the first pharmacologically active ingredient (A₁); and/or
segment (S₂) provides prolonged release of the second pharmacologically active ingredient (A₂); or
segment (S₂) provides immediate release of the second pharmacologically active ingredient (A₂); and/or
the first pharmacologically active ingredient (A₁) is embedded in a matrix material comprising a synthetic or natural polymer (C) selected from polyalkylene oxides or acrylic polymers; and/or
segment (S₁) and segment (S₂) are obtained from co-extrusion; and/or
the first pharmacologically active ingredient (A₁) has a psychotropic effect; and/or
the first pharmacologically active ingredient (A₁) is an opioid; and/or
the second pharmacologically active ingredient (A₂) has no psychotropic effect; or
the second pharmacologically active ingredient (A₂) has a psychotropic effect; and/or
the second pharmacologically active ingredient (A₂) is an NSAID; or
the second pharmacologically active ingredient (A₂) is an opioid; and/or
the monolithic pharmaceutical dosage form is a mantle tablet; and/or
the monolithic pharmaceutical dosage form consists of at least one first segment (S₁), at least one second segment (S₂) and optionally a film coating.

### EXAMPLES

### Example 1

A co-extrudate was manufactured which comprised a tamper-resistant inner phase containing an opioid and an outer phase containing an NSAID. The co-extrudate was cut into segments in order to yield the desired dosage.

TABLE 1
Formulation of the inner phase.

| component | wt.-% | m/mg |
|---|---|---|
| opioid (tramadol HCl) | 29.12 | 28.83 |
| hypromellose 100.000 mPa · s | 14.00 | 13.86 |
| polyethylene oxide 7.000.000 | 46.78 | 46.31 |
| PEG 6000 | 10.00 | 9.90 |
| alpha-tocopherol | 0.10 | 0.10 |
| total weight inner phase | 100.00 | 99.00 |
| (segment) | | |

The components were weighed, hand-sieved (mesh size 1.0 mm) and mixed in a container mixer (40 min, 6 rpm).

The powder mixture was extruded using a Leistritz extruder (ZSE27 PH 40D Micro) with a medium shear screw and a nozzle having a diameter d of 3 mm. Extrusion temperature profile: HZ1:25° C., HZ2:110° C., HZ3:105° C., HZ4:105° C., HZ5:100° C., HZ6:100° C., HZ7:90° C., HZ8:80° C., HZ9:40° C., HZ10:40° C., HZ11 (nozzle):135° C. Screw speed: 70 rpm. Dosing rate: 8.33 g/min=0.5 kg/h. The extruded strand of the inner phase was cooled by the ambient air and then cut into strands having a length of approx. 1 m.

The breaking strength (resistance to crushing) was measured using a Zwick Z 2.5 materials tester, Fₘₐₓ=2.5 kN with a maximum draw of 1150 mm. The inner phase exhibited a breaking strength of more than 500 N.

TABLE 2
Formulation of the outer phase.

| component | wt.-% | m/mg |
|---|---|---|
| paracetamol | 60.00 | 85.80 |
| Kollicoat ® IR | 30.00 | 42.90 |
| Lutrol ® F68 | 10.00 | 14.30 |
| total weight outer phase | 100.00 | 143.00 |
| (segment) | | |

The extruded strands of the inner phase were sheathed with the outer phase using a cable sheathing nozzle (inner diameter: 4 mm, outer diameter: 6 mm) and a twin screw extruder (Thermo Fisher Scientific Pharma 16 HME) with a medium shear screw. Extrusion temperature profile: HZ1:20° C., HZ2:120° C., HZ3:120° C., HZ4:120° C., HZ5:120° C., HZ6:120° C., HZ7:120° C., HZ8:120° C., HZ9 (adapter): 120° C., HZ10 (nozzle):130° C. Screw speed: 150 rpm. Dosing rate: 8.33 g/min=0.5 kg/h.

The extruded strand was cooled by the ambient air and then cut into segments having a total weight of 242.00 mg.

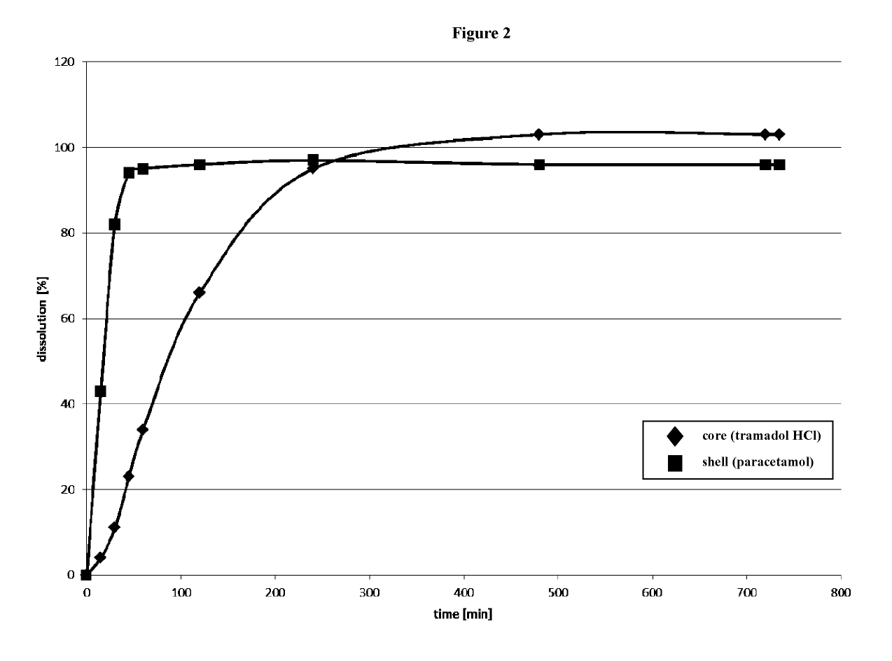

FIG. 2 shows the release profile of these segments (n=3) in 0.1 M HCl (pH=1, 900 mL, 50 rpm, paddle).

### Example 2

A co-extrudate was manufactured which comprised a tamper-resistant inner phase containing an opioid and an outer phase containing an NSAID. The co-extrudate was cut into segments in order to yield the desired dosage.

TABLE 3
Formulation of the inner phase.

| component | wt.-% | m/mg |
|---|---|---|
| opioid (tramadol HCl) | 29.12 | 28.83 |
| hypromellose 100.000 mPa · s | 14.00 | 13.86 |
| polyethylene oxide 7.000.000 | 46.78 | 46.31 |
| PEG 6000 | 10.00 | 9.90 |
| alpha-tocopherol | 0.10 | 0.10 |
| total weight inner phase | 100.00 | 99.00 |
| (segment) | | |

The components were weighed, hand-sieved (mesh size 1.0 mm) and mixed in a container mixer (40 min, 6 rpm).

The powder mixture was extruded using a Leistritz extruder (ZSE27 PH 40D Micro) with a medium shear screw and a nozzle having a diameter d of 3 mm. Extrusion temperature profile: HZ1:25° C., HZ2:110° C., HZ3:105° C., HZ4:105° C., HZ5:100° C., HZ6:100° C., HZ7:90° C., HZ8:80° C., HZ9:40° C., HZ10:40° C., HZ11 (nozzle):135° C. Screw speed: 70 rpm. Dosing rate: 8.33 g/min=0.5 kg/h. The extruded strand of the inner phase was cooled by the ambient air and then cut into strands having a length of approx. 1 m.

The breaking strength (resistance to crushing) was measured using a Zwick Z 2.5 materials tester, Fₘₐₓ=2.5 kN with a maximum draw of 1150 mm. The inner phase exhibited a breaking strength of more than 500 N.

TABLE 4
Formulation of the outer phase.

| component | wt.-% | m/mg |
|---|---|---|
| paracetamol | 50.00 | 76.00 |
| Kollicoat ® IR | 35.00 | 53.20 |
| PEG 6000 | 15.00 | 22.80 |
| total weight outer phase | 100.00 | 152.00 |
| (segment) | | |

The extruded strands of the inner phase were sheathed with the outer phase using a cable sheathing nozzle (inner diameter: 4 mm, outer diameter: 6 mm) and a twin screw extruder (Thermo Fisher Scientific Pharma 16 HME) with a medium shear screw. Extrusion temperature profile: HZ1:20° C., HZ2:120° C., HZ3:140° C., HZ4:140° C., HZ5:140° C., HZ6:140° C., HZ7:140° C., HZ8:140° C., HZ9 (adapter): 140° C., HZ11 (nozzle):145° C. Screw speed: 150 rpm. Dosing rate: 13.33 g/min=0.8 kg/h.

The extruded strand was cooled by the ambient air and then cut into segments having a total weight of 251.00 mg.

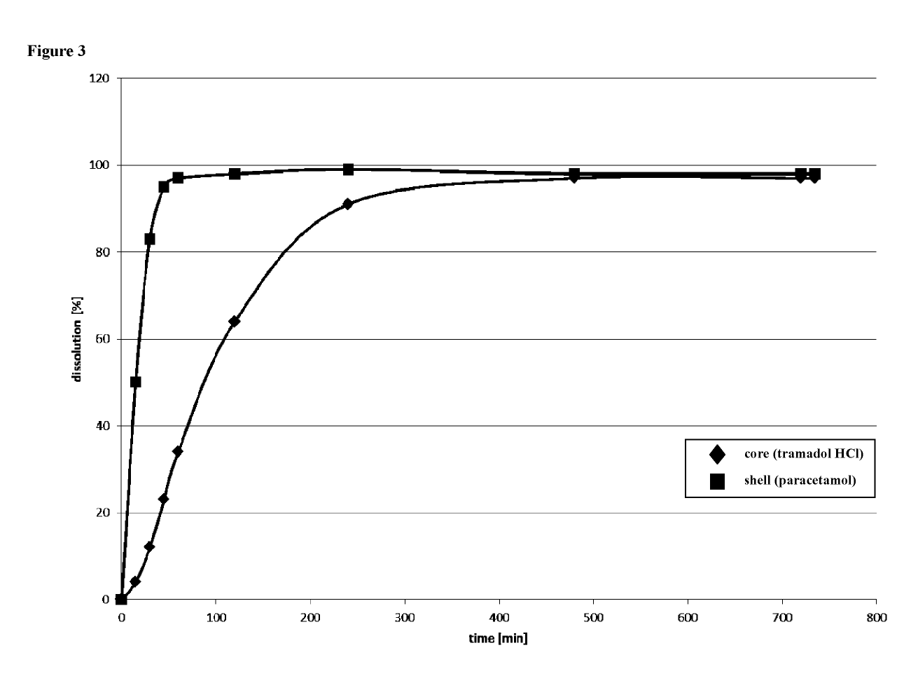

FIG. 3 shows the release profile of these segments (n=3) in 0.1 M HCl (pH=1, 900 mL, 50 rpm, paddle).

### Example 3

A co-extrudate was manufactured which comprised a tamper-resistant inner phase containing an opioid and an outer phase containing an NSAID. The co-extrudate was cut into segments in order to yield the desired dosage.

TABLE 5
Formulation of the inner phase.

| component | wt.-% | m/mg |
|---|---|---|
| opioid (tramadol HCl) | 2.33 | 2.64 |
| hypromellose 100.000 mPa · s | 10.00 | 11.34 |
| polyethylene oxide 7.000.000 | 70.00 | 79.35 |
| PEG 6000 | 16.80 | 19.04 |
| alpha-tocopherol | 0.03 | 0.03 |
| citric acid (anhydrous) | 0.84 | 0.95 |
| total weight inner phase | 100.00 | 113.35 |
| (segment) | | |

The components were weighed, sieved (Bohle BTS sieve, mesh size 1.0 mm, 250 rpm) and mixed in a free-fall mixer (15 min, 14 rpm).

The powder mixture was extruded using a Leistritz extruder (ZSE27 PH 40D Micro) with a medium shear screw and a nozzle having a diameter d of 3 mm. Extrusion temperature profile: HZ1:25° C., HZ2:110° C., HZ3:105° C., HZ4:105° C., HZ5:100° C., HZ6:100° C., HZ7:90° C., HZ8:80° C., HZ9:40° C., HZ10:40° C., HZ11 (nozzle):135° C. Screw speed: 100 rpm. Dosing rate: 8.33 g/min=0.5 kg/h. The extruded strand of the inner phase was cooled by the ambient air and then cut into strands having a length of approx. 1 m.

The breaking strength (resistance to crushing) was measured using a Zwick Z 2.5 materials tester, Fₘₐₓ=2.5 kN with a maximum draw of 1150 mm. The inner phase exhibited a breaking strength of more than 500 N.

TABLE 6
Formulation of the outer phase.

| component | wt.-% | m/mg |
|---|---|---|
| paracetamol | 80.00 | 130.68 |
| Eudragit ® E PO | 20.00 | 32.67 |
| total weight outer phase | 100.00 | 163.35 |
| (segment) | | |

The extruded strands of the inner phase were sheathed with the outer phase using a cable sheathing nozzle (inner diameter: 4 mm, outer diameter: 6 mm) and a twin screw extruder (ZSE18 HP PH 40D) with a medium shear screw. Extrusion temperature profile: HZ1:25° C., HZ2:105° C., HZ3:110° C., HZ4:140° C., HZ5:140° C., HZ6:140° C., HZ7:140° C., HZ8:140° C., HZ10:140° C., HZ11 (nozzle):135° C. Screw speed: 100 rpm. Dosing rate: 13.33 g/min=0.8 kg/h.

The extruded strand was cooled by the ambient air and then cut into segments having a total weight of 276.70 mg.

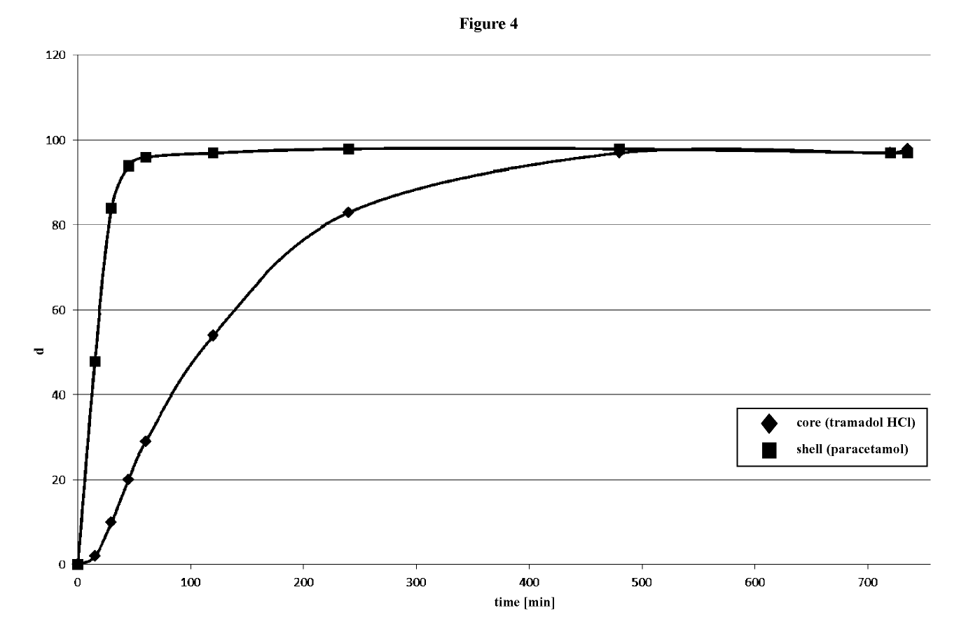

FIG. 4 shows the release profile of these segments (n=3) in 0.1 M HCl (pH=1, 900 mL, 50 rpm, paddle).

### Example 4

A co-extrudate was manufactured which comprised a tamper-resistant inner phase containing an opioid and an outer phase containing an NSAID. The co-extrudate was cut into segments in order to yield the desired dosage.

TABLE 7
Formulation of the inner phase.

| component | wt.-% | m/mg |
|---|---|---|
| opioid (tramadol HCl) | 2.33 | 2.64 |
| hypromellose 100.000 mPa · s | 10.00 | 11.34 |
| polyethylene oxide 7.000.000 | 70.00 | 79.35 |
| PEG 6000 | 16.80 | 19.04 |
| alpha-tocopherol | 0.03 | 0.03 |
| citric acid (anhydrous) | 0.84 | 0.95 |
| total weight inner phase | 100.00 | 113.35 |
| (segment) | | |

The components were weighed, sieved (Bohle BTS sieve, mesh size 1.0 mm, 250 rpm) and mixed in a free-fall mixer (15 min, 14 rpm).

The powder mixture was extruded using a Leistritz extruder (ZSE27 PH 40D Micro) with a medium shear screw and a nozzle having a diameter d of 3 mm. Extrusion temperature profile: HZ1:25° C., HZ2:110° C., HZ3:105° C., HZ4:105° C., HZ5:100° C., HZ6:100° C., HZ7:90° C., HZ8:80° C., HZ9:40° C., HZ10:40° C., HZ11 (nozzle):135° C. Screw speed: 100 rpm. Dosing rate: 8.33 g/min=0.5 kg/h. The extruded strand of the inner phase was cooled by the ambient air and then cut into strands having a length of approx. 1 m.

The breaking strength (resistance to crushing) was measured using a Zwick Z 2.5 materials tester, Fₘₐₓ=2.5 kN with a maximum draw of 1150 mm. The inner phase exhibited a breaking strength of more than 500 N.

TABLE 8
Formulation of the outer phase.

| component | wt.-% | m/mg |
|---|---|---|
| paracetamol | 60.00 | 104.01 |
| Eudragit ® E PO | 35.00 | 60.67 |
| stearic acid | 5.00 | 8.67 |
| total weight outer phase | 100.00 | 173.35 |
| (segment) | | |

The extruded strands of the inner phase were sheathed with the outer phase using a cable sheathing nozzle (inner diameter: 4 mm, outer diameter: 6 mm) and a twin screw extruder (ZSE18 HP PH 40D) with a medium shear screw. Extrusion temperature profile: HZ1:25° C., HZ2:105° C., HZ3:100° C., HZ4:90° C., HZ5:90° C., HZ6:85° C., HZ7:85° C., HZ8:50° C., HZ10:50° C., HZ11 (nozzle):135° C. Screw speed: 100 rpm. Dosing rate: 12.5 g/min=0.75 kg/h.

The extruded strand was cooled by the ambient air and then cut into segments having a total weight of 286.70 mg.

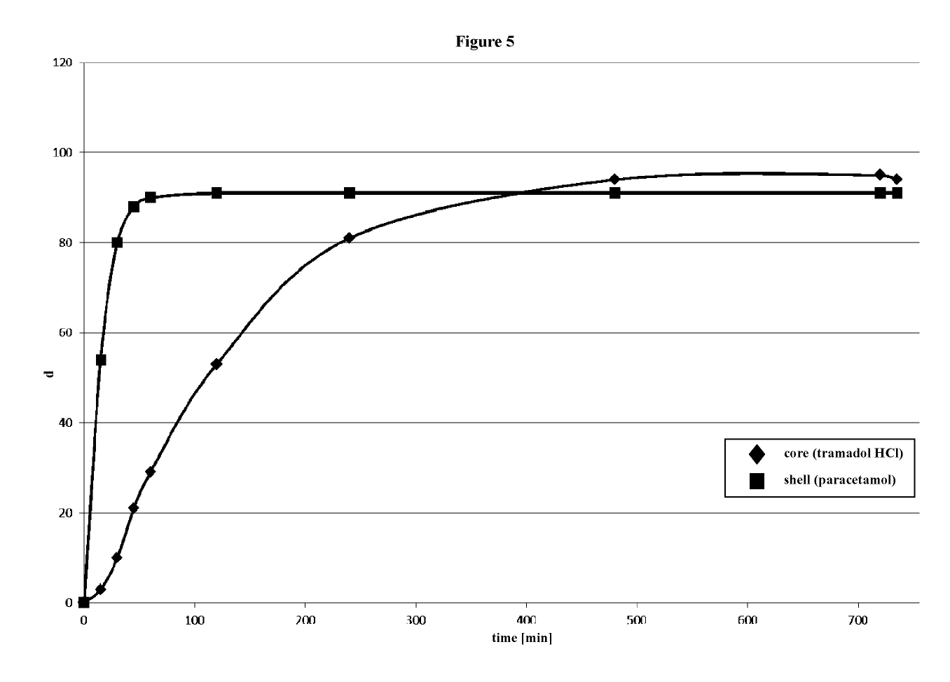

FIG. 5 shows the release profile of these segments (n=3) in 0.1 M HCl (pH=1, 900 mL, 50 rpm, paddle).

### Example 5

A co-extrudate was manufactured which comprised a tamper-resistant inner phase containing an opioid and an outer phase containing an NSAID. The co-extrudate was cut into segments in order to yield the desired dosage.

TABLE 9
Formulation of the inner phase.

| component | wt.-% | m/mg |
|---|---|---|
| opioid (tramadol HCl) | 29.12 | 36.31 |
| hypromellose 100.000 mPa · s | 14.00 | 17.46 |
| polyethylene oxide 7.000.000 | 46.78 | 58.33 |
| PEG 6000 | 10.00 | 12.47 |
| alpha-tocopherol | 0.10 | 0.13 |
| total weight inner phase | 100.00 | 124.70 |
| (segment) | | |

The components were weighed, hand-sieved (mesh size 1.0 mm) and mixed in a container mixer (40 min, 6 rpm).

The powder mixture was extruded using a Leistritz extruder (ZSE27 PH 40D Micro) with a medium shear screw and a nozzle having a diameter d of 3 mm. Extrusion temperature profile: HZ1:25° C., HZ2:110° C., HZ3:105° C., HZ4:105° C., HZ5:100° C., HZ6:100° C., HZ7:90° C., HZ8:80° C., HZ9:40° C., HZ10:40° C., HZ11 (nozzle):135° C. Screw speed: 70 rpm. Dosing rate: 8.33 g/min=0.5 kg/h. The extruded strand of the inner phase was cooled by the ambient air and then cut into strands having a length of approx. 1 m.

The breaking strength (resistance to crushing) was measured using a Zwick Z 2.5 materials tester, Fₘₐₓ=2.5 kN with a maximum draw of 1150 mm. The inner phase exhibited a breaking strength of more than 500 N.

TABLE 10
Formulation of the outer phase.

| component | wt.-% | m/mg |
|---|---|---|
| paracetamol | 50.00 | 196.25 |
| Lutrol ® F68 | 30.00 | 117.75 |
| PEG 6000 | 20.00 | 78.50 |
| total weight outer phase | 100.00 | 392.50 |
| (segment) | | |

The extruded strands of the inner phase were manually sheathed with the outer phase which had been melted on a heating plate. The obtained sheathed strands were cooled by the ambient air and then cut into segments having a total weight of 517.20 mg.

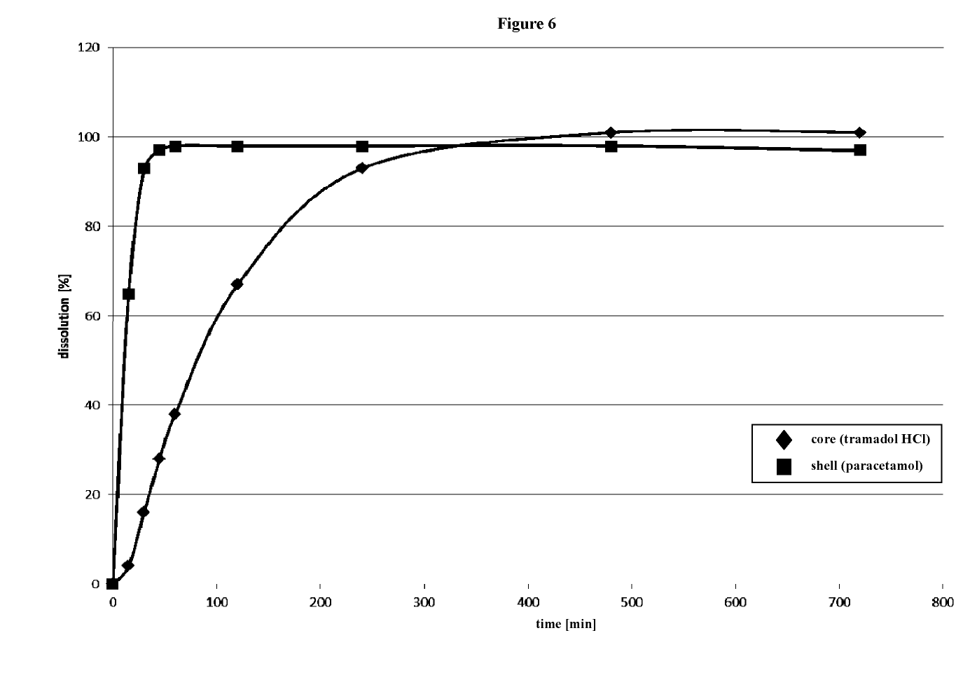

FIG. 6 shows the release profile of these segments tablets (n=3) in 0.1 M HCl (pH=1, 900 mL, 50 rpm, paddle).

### Example 6

A co-extrudate was manufactured which comprised a tamper-resistant inner phase containing an opioid and a tamper-resistant outer phase containing paracetamol. The co-extrudate was cut into segments in order to yield the desired dosage.

TABLE 11
Formulation of the inner phase.

| component | wt.-% | m/mg |
|---|---|---|
| opioid (tramado HCl) | 2.33 | 2.64 |
| hypromellose 100.000 mPa · s | 10.00 | 11.34 |
| polyethylene oxide 7.000.000 | 70.00 | 79.34 |
| PEG 6000 | 16.63 | 18.85 |
| alpha-tocopherol | 0.20 | 0.23 |
| citric acid (anhydrous) | 0.84 | 0.95 |
| total weight inner phase | 100.00 | 113.35 |
| (segment) | | |

The components were weighed, sieved (Bohle BTS sieve, mesh size 1.0 mm, 250 rpm) and mixed in a free-fall mixer (15 min, 14 rpm).

The powder mixture was extruded using a Leistritz extruder (ZSE27 PH 40D Micro) with a medium shear screw and a nozzle having a diameter d of 3 mm. Extrusion temperature profile: HZ1:25° C., HZ2:110° C., HZ3:105° C., HZ4:105° C., HZ5:100° C., HZ6:100° C., HZ7:90° C., HZ8:80° C., HZ9:40° C., HZ10:40° C., HZ11 (nozzle):135° C. Screw speed: 100 rpm. Dosing rate: 8.33 g/min=0.5 kg/h. The extruded strand of the inner phase was cooled by the ambient air and then cut into strands having a length of approx. 1 m.

The breaking strength (resistance to crushing) was measured using a Zwick Z 2.5 materials tester, Fₘₐₓ=2.5 kN with a maximum draw of 1150 mm. The inner phase exhibited a breaking strength of more than 500 N.

TABLE 12
Formulation of the outer phase.

| component | wt.-% | m/mg |
|---|---|---|
| paracetamol | 18.60 | 31.00 |
| hypromellose 100.000 mPa · s | 10.00 | 16.66 |
| polyethylene oxide 7.000.000 | 56.80 | 94.66 |
| PEG 6000 | 13.56 | 22.60 |
| alpha-tocopherol | 0.20 | 0.33 |
| citric acid (anhydrous) | 0.84 | 1.40 |
| total weight outer phase | 100.00 | 166.65 |
| (segment) | | |

The extruded strands of the inner phase were sheathed with the outer phase using a cable sheathing nozzle (inner diameter: 4 mm, outer diameter: 6 mm) and a twin screw extruder (ZSE18 HP PH 40D) with a medium shear screw. Extrusion temperature profile: HZ1:25° C., HZ2:105° C., HZ3:100° C., HZ4:90° C., HZ5:90° C., HZ6:85° C., HZ7:85° C., HZ8:50° C., HZ10:50° C., HZ11 (nozzle):135° C. Screw speed: 100 rpm. Dosing rate: 13.33 g/min=0.8 kg/h.

The outer phase exhibited a breaking strength of more than 500 N.

The extruded strand was cooled by the ambient air and then cut into segments which were formed into oblong tablets (6×15 mm) having a total weight of 280.00 mg.

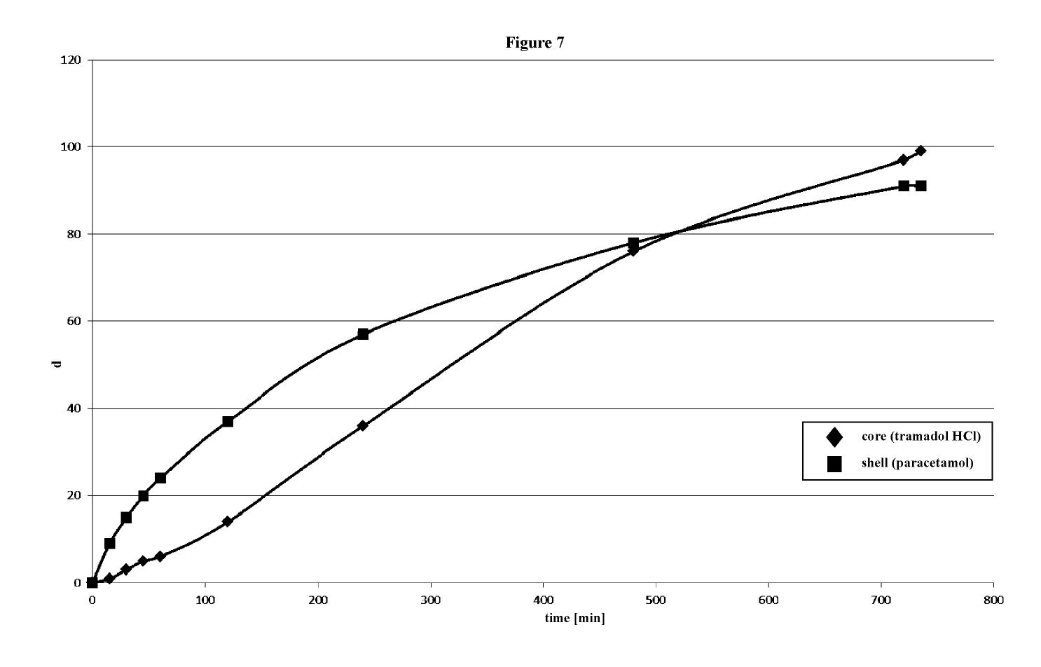

FIG. 7 shows the release profile of the tablets (n=3) in 0.1 M HCl (pH=1, 900 mL, 50 rpm, paddle).

### Example 7

A co-extrudate was manufactured which comprised a tamper-resistant inner phase containing an opioid and an outer phase (armoring layer, shelter layer) containing an NSAID. The co-extrudate was cut into segments in order to yield the desired dosage.

TABLE 13
Formulation of the inner phase.

| component | wt.-% | m/mg |
|---|---|---|
| opioid (tramadol HCl) | 2.33 | 2.64 |
| hypromellose 100.000 mPa · s | 10.00 | 11.34 |
| polyethylene oxide 7.000.000 | 70.00 | 79.35 |
| PEG 6000 | 16.80 | 19.04 |
| alpha-tocopherol | 0.03 | 0.03 |
| citric acid (anhydrous) | 0.84 | 0.95 |
| total weight inner phase | 100.00 | 113.35 |
| (segment) | | |

The components were weighed, sieved (Bohle BTS sieve, mesh size 1.0 mm, 250 rpm) and mixed in a free-fall mixer (15 min, 14 rpm).

The powder mixture was extruded using a Leistritz extruder (ZSE27 PH 40D Micro) with a medium shear screw and a nozzle having a diameter d of 3 mm. Extrusion temperature profile: HZ1:25° C., HZ2:110° C., HZ3:105° C., HZ4:105° C., HZ5:100° C., HZ6:100° C., HZ7:90° C., HZ8:80° C., HZ9:40° C., HZ10:40° C., HZ11 (nozzle):135° C. Screw speed: 100 rpm. Dosing rate: 8.33 g/min=0.5 kg/h. The extruded strand of the inner phase was cooled by the ambient air and then cut into strands having a length of approx. 1 m.

The breaking strength (resistance to crushing) was measured using a Zwick Z 2.5 materials tester, Fₘₐₓ=2.5 kN with a maximum draw of 1150 mm. The inner phase exhibited a breaking strength of more than 500 N.

TABLE 14
Formulation of the outer phase.

| component | wt.-% | m/mg |
|---|---|---|
| paracetamol | 70.00 | 156.07 |
| Eudragit ® FS 100 | 30.00 | 66.88 |
| total weight outer phase | 100.00 | 222.95 |
| (segment) | | |

The extruded strands of the inner phase were sheathed with the outer phase using a cable sheathing nozzle (inner diameter: 4 mm, outer diameter: 6 mm) and a twin screw extruder (ZSE18 HP PH 40D) with a medium shear screw. Extrusion temperature profile: HZ1:25° C., HZ2:105° C., HZ3:110° C., HZ4:140° C., HZ5:140° C., HZ6:140° C., HZ7:140° C., HZ8:140° C., HZ10:140° C., HZ11 (nozzle):135° C. Screw speed: 100 rpm. Dosing rate: 10.00 g/min=0.6 kg/h.

The extruded strand was cooled by the ambient air and then cut into segments having a total weight of 336.30 mg.

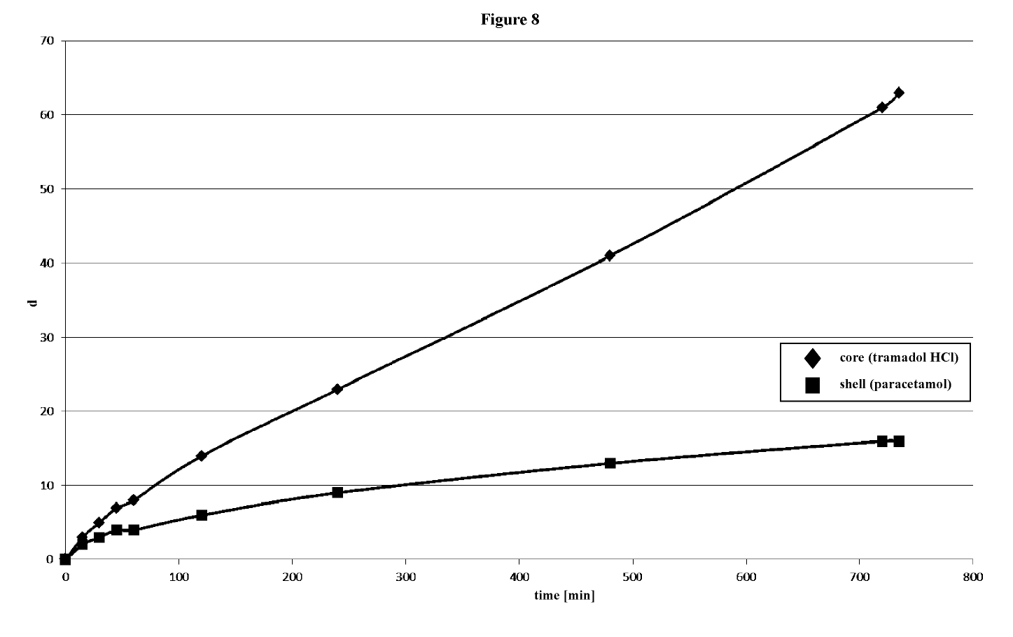

FIG. 8 shows the release profile of these segments (n=3) in 0.1 M HCl (pH=1, 900 mL, 50 rpm, paddle).

### Example 8

A co-extrudate was manufactured which comprised a tamper-resistant inner phase containing an opioid and an outer phase (armoring layer, shelter layer) containing an NSAID. The co-extrudate was cut into segments in order to yield the desired dosage.

TABLE 15
Formulation of the inner phase.

| component | wt.-% | m/mg |
|---|---|---|
| opioid (tramadol HCl) | 2.33 | 2.64 |
| hypromellose 100.000 mPa · s | 10.00 | 11.34 |
| polyethylene oxide 7.000.000 | 70.00 | 79.35 |
| PEG 6000 | 16.80 | 19.04 |
| alpha-tocopherol | 0.03 | 0.03 |
| citric acid (anhydrous) | 0.84 | 0.95 |
| total weight inner phase | 100.00 | 113.35 |
| (segment) | | |

The components were weighed, sieved (Bohle BTS sieve, mesh size 1.0 mm, 250 rpm) and mixed in a free-fall mixer (15 min, 14 rpm).

The powder mixture was extruded using a Leistritz extruder (ZSE27 PH 40D Micro) with a medium shear screw and a nozzle having a diameter d of 3 mm. Extrusion temperature profile: HZ1:25° C., HZ2:110° C., HZ3:105° C., HZ4:105° C., HZ5:100° C., HZ6:100° C., HZ7:90° C., HZ8:80° C., HZ9:40° C., HZ10:40° C., HZ11 (nozzle):135° C. Dosing rate: 8.33 g/min=0.5 kg/h. The extruded strand of the inner phase was cooled by the ambient air and then cut into strands having a length of approx. 1 m.

The breaking strength (resistance to crushing) was measured using a Zwick Z 2.5 materials tester, Fₘₐₓ=2.5 kN with a maximum draw of 1150 mm. The inner phase exhibited a breaking strength of more than 500 N.

TABLE 16
Formulation of the outer phase.

| component | wt.-% | m/mg |
|---|---|---|
| paracetamol | 67.74 | 106.12 |
| Oppanol ® B10 | 22.58 | 35.37 |
| Eudragit ® E100 | 9.68 | 15.16 |
| total weight outer phase | 100.00 | 156.65 |
| (segment) | | |

The extruded strands of the inner phase were sheathed with the outer phase using a cable sheathing nozzle (inner diameter: 4 mm, outer diameter: 6 mm) and a twin screw extruder (Thermo Fisher Scientific Pharma 16 HME) with a medium shear screw. Extrusion temperature profile: HZ1:20° C., HZ2:110° C., HZ3:110° C., HZ4:110° C., HZ5:110° C., HZ6:110° C., HZ7:110° C., HZ8:120° C., HZ9 (adapter): 130° C., HZ10 (nozzle):135° C. Screw speed: 100 rpm. Dosing rate: 8.33 g/min=0.5 kg/h.

The extruded strand was cooled by the ambient air and then cut into segments which were formed into oblong tablets (6×15 mm) having a total weight of 270.00 mg.

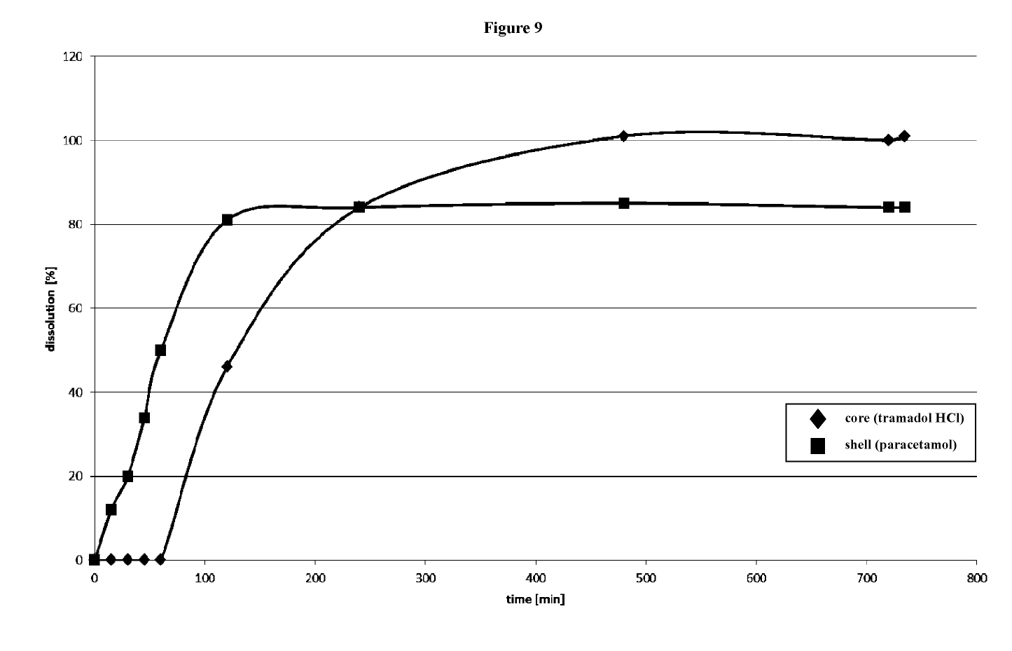

FIG. 9 shows the release profile of the tablets (n=3) in 0.1 M HCl (pH=1, 900 mL, 50 rpm, paddle).

### Example 9

A co-extrudate was manufactured which comprised a tamper-resistant inner phase containing an opioid and an outer phase (armoring layer, shelter layer) containing an NSAID. The co-extrudate was cut into segments in order to yield the desired dosage.

TABLE 17
Formulation of the inner phase.

| component | wt.-% | m/mg |
|---|---|---|
| opioid (tramadol HCl) | 29.12 | 28.83 |
| hypromellose 100.000 mPa · s | 14.00 | 13.86 |
| polyethylene oxide 7.000.000 | 46.78 | 46.31 |
| PEG 6000 | 10.00 | 9.90 |
| alpha-tocopherol | 0.10 | 0.10 |
| total weight inner phase | 100.00 | 99.00 |
| (segment) | | |

The components were weighed, hand-sieved (mesh size 1.0 mm) and mixed in a container mixer (40 min, 6 rpm).

The powder mixture was extruded using a Leistritz extruder (ZSE27 PH 40D Micro) with a medium shear screw and a nozzle having a diameter d of 3 mm. Extrusion temperature profile: HZ1:25° C., HZ2:110° C., HZ3:105° C., HZ4:105° C., HZ5:100° C., HZ6:100° C., HZ7:90° C., HZ8:80° C., HZ9:40° C., HZ10:40° C., HZ11 (nozzle):135° C. Screw speed: 70 rpm. Dosing rate: 8.33 g/min=0.5 kg/h. The extruded strand of the inner phase was cooled by the ambient air and then cut into strands having a length of approx. 1 m.

The breaking strength (resistance to crushing) was measured using a Zwick Z 2.5 materials tester, Fₘₐₓ=2.5 kN with a maximum draw of 1150 mm. The inner phase exhibited a breaking strength of more than 500 N.

TABLE 18
Formulation of the outer phase.

| component | wt.-% | m/mg |
|---|---|---|
| paracetamol | 50.00 | 148.00 |
| Kolliwax ® SA | 10.00 | 29.60 |
| PEG 6000 | 10.00 | 29.60 |
| Soluplus ® | 30.00 | 88.80 |
| total weight outer phase | 100.00 | 296.00 |
| (segment) | | |

The extruded strands of the inner phase were sheathed with the outer phase using a cable sheathing nozzle (inner diameter: 4 mm, outer diameter: 6 mm) and a twin screw extruder (Thermo Fisher Scientific Pharma 16 HME) with a medium shear screw. Extrusion temperature profile: HZ1:20° C., HZ2:30° C., HZ3:40° C., HZ4:90° C., HZ5:120° C., HZ6:50° C., HZ7:30° C., HZ8:30° C., HZ9 (adapter): 50° C., HZ10 (nozzle):100° C. Screw speed: 150 rpm. Dosing rate: 13.33 g/min=0.8 kg/h.

The extruded strand was cooled by the ambient air and then cut into segments which were formed into oblong tablets (6×15 mm) having a total weight of 395.00 mg.

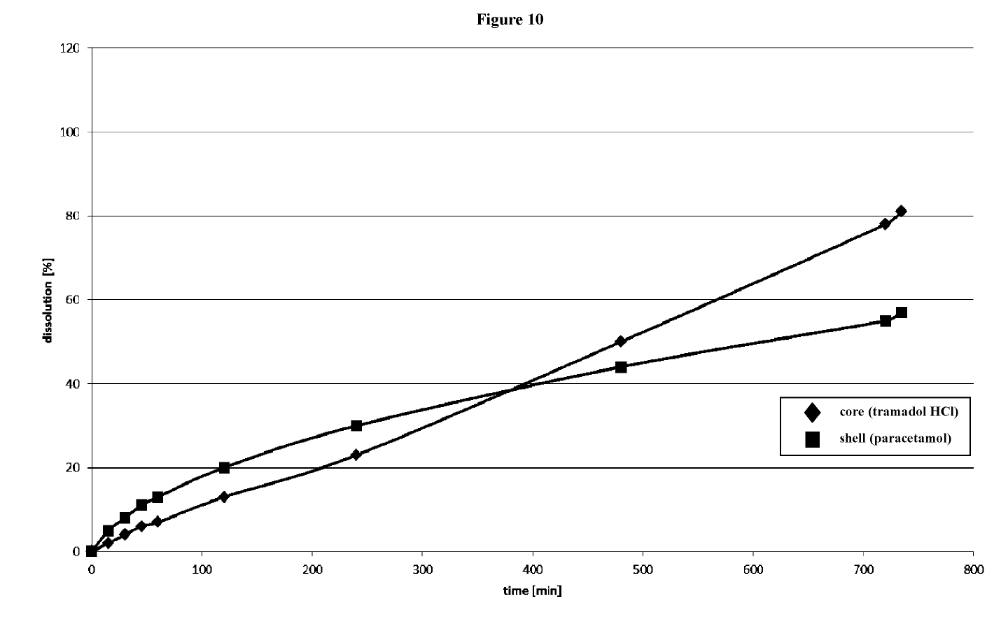

FIG. 10 shows the release profile of the tablets (n=3) in 0.1 M HCl (pH=1, 900 mL, 50 rpm, paddle).

### Example 10

A co-extrudate was manufactured which comprised a tamper-resistant inner phase containing an opioid and an outer phase containing an NSAID. The co-extrudate was cut into segments in order to yield the desired dosage.

TABLE 19
Formulation of the inner phase.

| component | wt.-% | m/mg |
|---|---|---|
| opioid (tramadol HCl) | 29.12 | 28.83 |
| hypromellose 100.000 mPa · s | 14.00 | 13.86 |
| polyethylene oxide 7.000.000 | 46.78 | 46.31 |
| PEG 6000 | 10.00 | 9.90 |
| alpha-tocopherol | 0.10 | 0.10 |
| total weight inner phase | 100.00 | 99.00 |
| (segment) | | |

The components were weighed, hand-sieved (mesh size 1.0 mm) and mixed in a container mixer (40 min, 6 rpm).

The powder mixture was extruded using a Leistritz extruder (ZSE27 PH 40D Micro) with a medium shear screw and a nozzle having a diameter d of 3 mm. Extrusion temperature profile: HZ1:25° C., HZ2:110° C., HZ3:105° C., HZ4:105° C., HZ5:100° C., HZ6:100° C., HZ7:90° C., HZ8:80° C., HZ9:40° C., HZ10:40° C., HZ11 (nozzle):135° C. Screw speed: 70 rpm. Dosing rate: 8.33 g/min=0.5 kg/h. The extruded strand of the inner phase was cooled by the ambient air and then cut into strands having a length of approx. 1 m.

The breaking strength (resistance to crushing) was measured using a Zwick Z 2.5 materials tester, Fₘₐₓ=2.5 kN with a maximum draw of 1150 mm. The inner phase exhibited a breaking strength of more than 500 N.

TABLE 20
Formulation of the outer phase.

| component | wt.-% | m/mg |
|---|---|---|
| paracetamol | 45.00 | 138.60 |
| Soluplus ® | 30.00 | 92.40 |
| Kolliwax ® SA | 10.00 | 30.80 |
| PEG 6000 | 10.00 | 30.80 |
| ascorbic acid | 5.00 | 15.40 |
| total weight outer phase | 100.00 | 308.00 |
| (segment) | | |

The extruded strands of the inner phase were sheathed with the outer phase using a cable sheathing nozzle (inner diameter: 4 mm, outer diameter: 6 mm) and a twin screw extruder (Thermo Fisher Scientific Pharma 16 HME) with a medium shear screw. Extrusion temperature profile: HZ1:20° C., HZ2:50° C., HZ3:50° C., HZ4:90° C., HZ5:120° C., HZ6:50° C., HZ7:35° C., HZ8:30° C., HZ9 (adapter): 45° C., HZ10 (nozzle):115° C. Screw speed: 200 rpm. Dosing rate: 25.00 g/min=1.5 kg/h.

The extruded strand was cooled by the ambient air and then cut into segments which were formed into oblong tablets (6×15 mm) having a total weight of 407.00 mg.

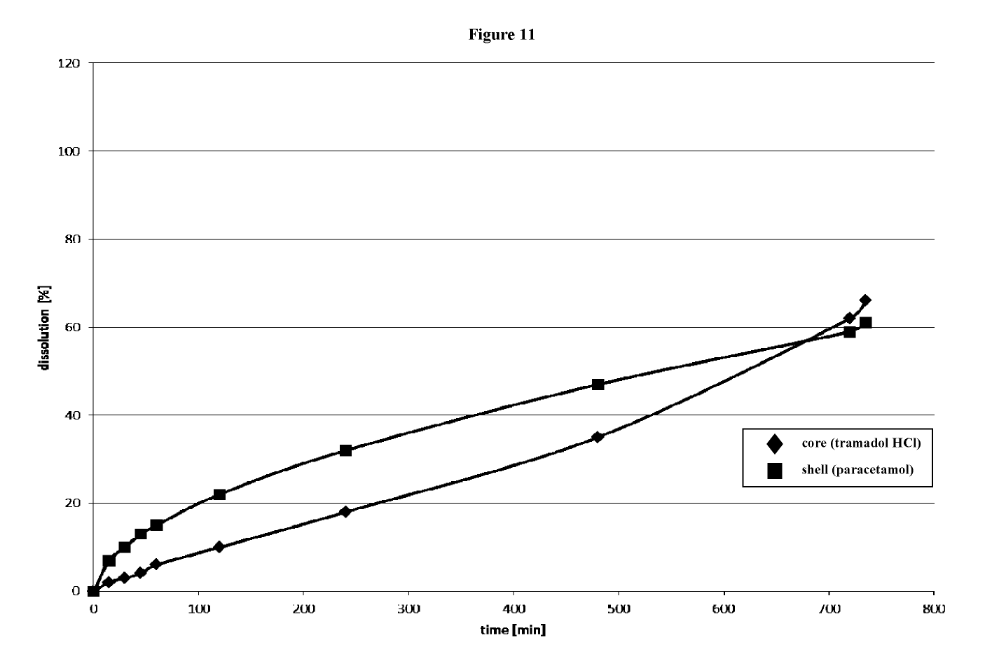

FIG. 11 shows the release profile of the tablets (n=3) in 0.1 M HCl (pH=1, 900 mL, 50 rpm, paddle).

### Example 11

A co-extrudate was manufactured which comprised a tamper-resistant inner phase containing an opioid and an outer phase containing an NSAID. The co-extrudate was cut into segments in order to yield the desired dosage.

TABLE 21
Formulation of the inner phase.

| component | wt.-% | m/mg |
|---|---|---|
| opioid (tramadol HCl) | 29.12 | 28.83 |
| hypromellose 100.000 mPa · s | 14.00 | 13.86 |
| polyethylene oxide 7.000.000 | 46.78 | 46.31 |
| PEG 6000 | 10.00 | 9.90 |
| alpha-tocopherol | 0.10 | 0.10 |
| total weight inner phase | 100.00 | 99.00 |
| (segment) | | |

The components were weighed, hand-sieved (mesh size 1.0 mm) and mixed in a container mixer (40 min, 6 rpm).

The powder mixture was extruded using a Leistritz extruder (ZSE27 PH 40D Micro) with a medium shear screw and a nozzle having a diameter d of 3 mm. Extrusion temperature profile: HZ1:25° C., HZ2:110° C., HZ3:105° C., HZ4:105° C., HZ5:100° C., HZ6:100° C., HZ7:90° C., HZ8:80° C., HZ9:40° C., HZ10:40° C., HZ11 (nozzle):135° C. Screw speed: 70 rpm. Dosing rate: 8.33 g/min=0.5 kg/h. The extruded strand of the inner phase was cooled by the ambient air and then cut into strands having a length of approx. 1 m.

The breaking strength (resistance to crushing) was measured using a Zwick Z 2.5 materials tester, Fₘₐₓ=2.5 kN with a maximum draw of 1150 mm. The inner phase exhibited a breaking strength of more than 500 N.

TABLE 22
Formulation of the outer phase.

| component | wt.-% | m/mg |
|---|---|---|
| paracetamol | 40.00 | 88.00 |
| Compritol ® 888 | 20.00 | 44.00 |
| PEG 6000 | 10.00 | 22.00 |
| isomalt | 30.00 | 66.00 |
| total weight outer phase | 100.00 | 220.00 |
| (segment) | | |

The extruded strands of the inner phase were sheathed with the outer phase using a cable sheathing nozzle (inner diameter: 4 mm, outer diameter: 6 mm) and a twin screw extruder (Thermo Fisher Scientific Pharma 16 HME) with a medium shear screw. Extrusion temperature profile: HZ1:25° C., HZ2:70° C., HZ3:70° C., HZ4:70° C., HZ5:70° C., HZ6:70° C., HZ7:70° C., HZ8:70° C., HZ9 (adapter): 70° C., HZ10 (nozzle):105° C. Screw speed: 150 rpm. Dosing rate: 13.33 g/min=0.8 kg/h.

The extruded strand was cooled by the ambient air and then cut into segments which were formed into oblong tablets (6×15 mm) having a total weight of 319.00 mg.

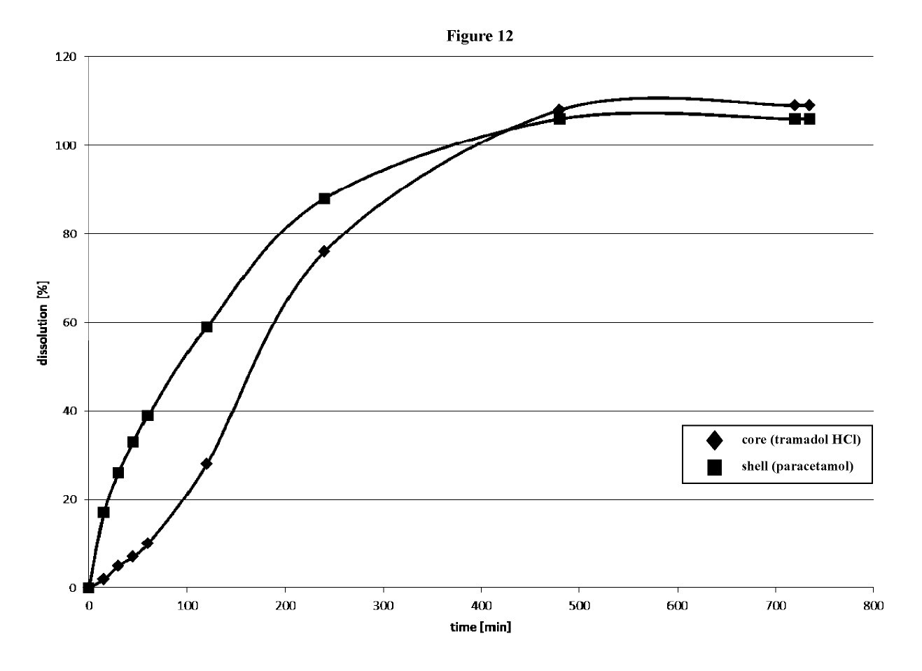

FIG. 12 shows the release profile of the tablets (n=3) in 0.1 M HCl (pH=1, 900 mL, 50 rpm, paddle).

### Example 12

A co-extrudate was manufactured which comprised a tamper-resistant inner phase containing an opioid and an outer phase containing an NSAID. The co-extrudate was cut into segments in order to yield the desired dosage.

TABLE 23
Formulation of the inner phase.

| component | wt.-% | m/mg |
|---|---|---|
| opioid (tramadol HCl) | 29.12 | 28.83 |
| hypromellose 100.000 mPa · s | 14.00 | 13.86 |
| polyethylene oxide 7.000.000 | 46.78 | 46.31 |
| PEG 6000 | 10.00 | 9.90 |
| alpha-tocopherol | 0.10 | 0.10 |
| total weight inner phase | 100.00 | 99.00 |
| (segment) | | |

The components were weighed, hand-sieved (mesh size 1.0 mm) and mixed in a container mixer (40 min, 6 rpm).

The powder mixture was extruded using a Leistritz extruder (ZSE27 PH 40D Micro) with a medium shear screw and a nozzle having a diameter d of 3 mm. Extrusion temperature profile: HZ1:25° C., HZ2:110° C., HZ3:105° C., HZ4:105° C., HZ5:100° C., HZ6:100° C., HZ7:90° C., HZ8:80° C., HZ9:40° C., HZ10:40° C., HZ11 (nozzle):135° C. Screw speed: 70 rpm. Dosing rate: 8.33 g/min=0.5 kg/h. The extruded strand of the inner phase was cooled by the ambient air and then cut into strands having a length of approx. 1 m.

The breaking strength (resistance to crushing) was measured using a Zwick Z 2.5 materials tester, Fₘₐₓ=2.5 kN with a maximum draw of 1150 mm. The inner phase exhibited a breaking strength of more than 500 N.

TABLE 24
Formulation of the outer phase.

| component | wt.-% | m/mg |
|---|---|---|
| paracetamol | 30.00 | 39.30 |
| Carbopol ® 71G | 30.00 | 39.30 |
| Lutrol ® F68 | 20.00 | 26.20 |
| PEG 6000 | 15.00 | 19.65 |
| ascorbic acid | 5.00 | 6.55 |
| total weight outer phase | 100.00 | 131.00 |
| (segment) | | |

The extruded strands of the inner phase were sheathed with the outer phase using a cable sheathing nozzle (inner diameter: 4 mm, outer diameter: 6 mm) and a twin screw extruder (Thermo Fisher Scientific Pharma 16 HME) with a medium shear screw. Extrusion temperature profile: HZ1:20° C., HZ2:100° C., HZ3:100° C., HZ4:105° C., HZ5:100° C., HZ6:100° C., HZ7:100° C., HZ8:100° C., HZ9 (adapter): 120° C., HZ10 (nozzle):120° C. Screw speed: 150 rpm. Dosing rate: 16.66 g/min=1.0 kg/h.

The extruded strand was cooled by the ambient air and then cut into segments having a total weight of 230.00 mg.

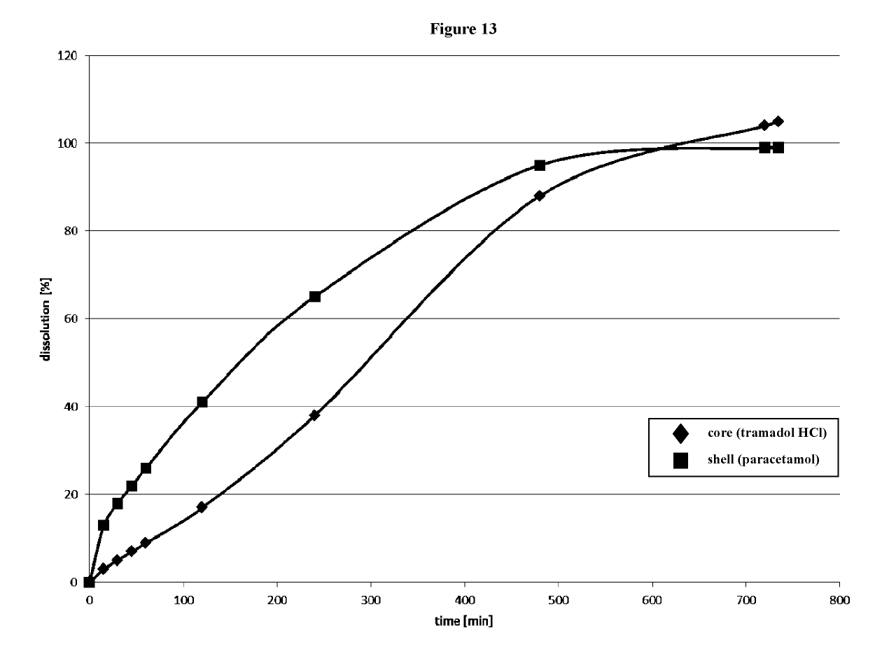

FIG. 13 shows the release profile of these segments (n=3) in 0.1 M HCl (pH=1, 900 mL, 50 rpm, paddle).

1. A monolithic pharmaceutical dosage form comprising:
a hot melt-extruded first segment (S₁); and
a second segment (S₂);
wherein
the first segment (S₁) contains at least a first pharmacologically active ingredient (A₁) and/or the second segment (S₂) contains at least a second pharmacologically active ingredient (A₂); and
the segment (S₁) and/or the segment (S₂) is tamper-resistant and/or exhibits a breaking strength of at least 300 N.

2. The monolithic pharmaceutical dosage form according to claim 1, comprising
a hot melt-extruded first segment (S₁) containing a first pharmacologically active ingredient (A₁); and
a hot melt-extruded second segment (S₂) containing a second pharmacologically active ingredient (A₂);
wherein
the segment (S₁) and/or the segment (S₂) is tamper-resistant and/or exhibits a breaking strength of at least 300 N; and
the segment (S₁) and/or the segment (S₂) provides prolonged release of the pharmacologically active ingredient (A₁) or (A₂) contained therein.

3. The monolithic pharmaceutical dosage form according to claim 1, wherein the second segment (S₂) covers at least a part of the surface of the first segment (S₁).

4. The monolithic pharmaceutical dosage form according to claim 1, which is a layered tablet.

5. The monolithic pharmaceutical dosage form according to claim 1, wherein the second segment (S₂) covers the entire surface of the first segment (S₁).

6. The monolithic pharmaceutical dosage form according to claim 1, which is a mantle tablet.

7. The monolithic pharmaceutical dosage form according to claim 1, wherein the relative weight ratio of the first segment (S₁) to the second segment (S₂) is within the range of from 90:10 to 10:90.

8. The monolithic pharmaceutical dosage form according to claim 1, having an overall breaking strength of at least 300 N.

9. The monolithic pharmaceutical dosage form according to claim 1, wherein the segment (S₁) and/or the segment (S₂) which is tamper-resistant and/or exhibits a breaking strength of at least 300 N provides resistance against grinding and/or resistance against solvent extraction and/or resistance against dose-dumping in aqueous ethanol.

10. The monolithic pharmaceutical dosage form according to claim 9, wherein the segment (S₁) and/or the segment (S₂) contains a pharmacologically active ingredient (A₁) and (A₂), respectively, which is embedded in a matrix material comprising a synthetic or natural polymer (C), wherein
the content of the synthetic or natural polymer (C) is at least 30 wt.-% relative to the total weight of the segment (S₁) and (S₂), respectively; and/or the synthetic or natural polymer (C) is selected from polyalkylene oxides or acrylic polymers.

11. The monolithic pharmaceutical dosage form according to claim 1, which consists of:
(i) at least one first segment (S₁) containing a first pharmacologically active ingredient (A₁);
(ii) at least one second segment (S₂) containing a second pharmacologically active ingredient (A₂); and
(iii) optionally a film coating.

12. A monolithic pharmaceutical dosage form according to claim 1, wherein
(a) the first segment (S₁) exhibits a breaking strength of at least 300 N and provides prolonged release of the first pharmacologically active ingredient (A₁) contained therein, whereby said first pharmacologically active ingredient (A₁) is an opioid; and
(a1) the second segment (S₂) exhibits a lower breaking strength than the first segment (S₁) and provides prolonged release of the second pharmacologically active ingredient (A₂) contained therein, whereby said second pharmacologically active ingredient (A₂) is an NSAID; or
(a2) the second segment (S₂) exhibits a lower breaking strength than the first segment (S₁) and provides immediate release of the second pharmacologically active ingredient (A₂) contained therein, whereby said second pharmacologically active ingredient (A₂) is an NSAID; or
(a3) the second segment (S₂) exhibits a breaking strength of at least 300 N and provides prolonged release of the second pharmacologically active ingredient (A₂) contained therein, whereby said second pharmacologically active ingredient (A₂) is an NSAID; or
(a4) the second segment (S₂) exhibits a breaking strength of at least 300 N and provides immediate release of the second pharmacologically active ingredient (A₂) contained therein, whereby said second pharmacologically active ingredient (A₂) is an NSAID; or
(a5) the second segment (S₂) exhibits a breaking strength of at least 300 N and provides prolonged release of the second pharmacologically active ingredient (A₂) contained therein, whereby said second pharmacologically active ingredient (A₂) is identical to the first pharmacologically active ingredient (A₁); or
(a6) the second segment (S₂) exhibits a breaking strength of at least 300 N and provides immediate release of the second pharmacologically active ingredient (A₂) contained therein, whereby said second pharmacologically active ingredient (A₂) is identical to the first pharmacologically active ingredient (A₁); or
(a7) the second segment (S₂) exhibits a breaking strength of at least 300 N and provides prolonged release of the second pharmacologically active ingredient (A₂) contained therein, whereby said second pharmacologically active ingredient (A₂) is an opioid which is different from the first pharmacologically active ingredient (A₁); or
(a8) the second segment (S₂) exhibits a breaking strength of at least 300 N and provides immediate release of the second pharmacologically active ingredient (A₂) contained therein, whereby said second pharmacologically active ingredient (A₂) is an opioid which is different from the first pharmacologically active ingredient (A₁);
or
(b) the second segment (S₂) exhibits a breaking strength of at least 300 N and provides prolonged release of the second pharmacologically active ingredient (A₂) contained therein, whereby said second pharmacologically active ingredient (A₂) is an opioid; and
(b1) the first segment (S₁) exhibits a lower breaking strength than the second segment (S₂) and provides prolonged release of the first pharmacologically active ingredient (A₁) contained therein, whereby said first pharmacologically active ingredient (A₁) is an NSAID; or
(b2) the first segment (S₁) exhibits a lower breaking strength than the second segment (S₂) and provides immediate release of the first pharmacologically active ingredient (A₁) contained therein, whereby said first pharmacologically active ingredient (A₁) is an NSAID; or
(b3) the first segment (S₁) exhibits a breaking strength of at least 300 N and provides prolonged release of the first pharmacologically active ingredient (A₁) contained therein, whereby said first pharmacologically active ingredient (A₁) is an NSAID; or
(b4) the first segment (S₁) exhibits a breaking strength of at least 300 N and provides immediate release of the first pharmacologically active ingredient (A₁) contained therein, whereby said first pharmacologically active ingredient (A₁) is an NSAID.

13. A monolithic pharmaceutical dosage form according to claim 1, which is a mantle tablet, wherein the relative weight ratio of the first segment (S₁) to the second segment (S₂) is within the range of from 1:1 to 1:3.5; and
(a) the first segment (S₁) exhibits a breaking strength of at least 500 N and provides prolonged release of the first pharmacologically active ingredient (A₁) contained therein, whereby said first pharmacologically active ingredient (A₁) is an opioid; and
(a1) the second segment (S₂) exhibits a lower breaking strength than the first segment (S₁) and provides prolonged release of the second pharmacologically active ingredient (A₂) contained therein, whereby said second pharmacologically active ingredient (A₂) is an NSAID; or
(a2) the second segment (S₂) exhibits a lower breaking strength than the first segment (S₁) and provides immediate release of the second pharmacologically active ingredient (A₂) contained therein, whereby said second pharmacologically active ingredient (A₂) is an NSAID; or
(a3) the second segment (S₂) exhibits a breaking strength of at least 300 N and provides prolonged release of the second pharmacologically active ingredient (A₂) contained therein, whereby said second pharmacologically active ingredient (A₂) is an NSAID.

14. A method of treating pain in patient in need of such treatment, said method comprising administering to said patient a monolithic pharmaceutical dosage form according to claim 1, wherein said monolithic pharmaceutical dosage form comprises at least one active ingredient effective therefor.

15. A process for the production of a monolithic pharmaceutical dosage form according to claim 1 comprising the steps of
(i) hot melt-extruding a first segment (S₁) containing a first pharmacologically active ingredient (A₁); and
(ii) hot melt-extruding a second segment (S₂) containing a second pharmacologically active ingredient (A₂);
wherein step (i) is performed before, after and/or simultaneously with step (ii).
